SEQUENCE LISTING (129 sequences)

SEQ ID NO: 1 - RNA, 20 nt, synthetic construct
  source 1..20: mol_type = other RNA
gcacctgaataccacgcctg

SEQ ID NO: 2 - RNA, 20 nt, synthetic construct
  source 1..20: mol_type = other RNA
cgcctgcgatgtagtcgatg

SEQ ID NO: 3 - RNA, 20 nt, synthetic construct
  source 1..20: mol_type = other RNA
caggacgggcgagatgtccc

SEQ ID NO: 4 - RNA, 20 nt, synthetic construct
  source 1..20: mol_type = other RNA
ctgaatctttggagtacctg

SEQ ID NO: 5 - RNA, 20 nt, synthetic construct
  source 1..20: mol_type = other RNA
ggccacggagcgagacatct

SEQ ID NO: 6 - RNA, 20 nt, synthetic construct
  source 1..20: mol_type = other RNA
aagtcaacttcaatgtcgga

SEQ ID NO: 7 - RNA, 20 nt, synthetic construct
  source 1..20: mol_type = other RNA
gcttggaggcctgatcagcg

SEQ ID NO: 8 - RNA, 20 nt, synthetic construct
  source 1..20: mol_type = other RNA
cttatctcttcgcagcgagg

SEQ ID NO: 9 - RNA, 20 nt, synthetic construct
  source 1..20: mol_type = other RNA
cacacattactccaacattg

SEQ ID NO: 10 - RNA, 20 nt, synthetic construct
  source 1..20: mol_type = other RNA
ttccgcaaaatagagcccca

SEQ ID NO: 11 - RNA, 20 nt, synthetic construct
  source 1..20: mol_type = other RNA
tgcacagaactatcgtacca

SEQ ID NO: 12 - RNA, 20 nt, synthetic construct
  source 1..20: mol_type = other RNA
gcaataagactctttaaaga

SEQ ID NO: 13 - RNA, 20 nt, synthetic construct
  source 1..20: mol_type = other RNA
caaagagattacgaatgcct

SEQ ID NO: 14 - RNA, 20 nt, synthetic construct
  source 1..20: mol_type = other RNA
caaggcaccccaggtttcca

SEQ ID NO: 15 - RNA, 20 nt, synthetic construct
  source 1..20: mol_type = other RNA
ttacgaatgccttggaaacc

SEQ ID NO: 16 - RNA, 20 nt, synthetic construct
  source 1..20: mol_type = other RNA
cagagacgcatctgaccctc

SEQ ID NO: 17 - RNA, 20 nt, synthetic construct
  source 1..20: mol_type = other RNA
catgcagttctcacacactg

SEQ ID NO: 18 - RNA, 20 nt, synthetic construct
  source 1..20: mol_type = other RNA
gtgtgagaactgcatggaga

SEQ ID NO: 19 - RNA, 20 nt, synthetic construct
  source 1..20: mol_type = other RNA
tctcatttcaggaaaccact

SEQ ID NO: 20 - RNA, 20 nt, synthetic construct
  source 1..20: mol_type = other RNA
agtcatacaccttaaccaag

SEQ ID NO: 21 - RNA, 20 nt, synthetic construct
  source 1..20: mol_type = other RNA
ttcaaggaaaccagttgagg

SEQ ID NO: 22 - RNA, 20 nt, synthetic construct
  source 1..20: mol_type = other RNA
gagccttgcctggaaatctg

SEQ ID NO: 23 - RNA, 20 nt, synthetic construct
  source 1..20: mol_type = other RNA
aagcgtcaaaagtctgccag

SEQ ID NO: 24 - RNA, 20 nt, synthetic construct
  source 1..20: mol_type = other RNA
cgttccaactcgaagtgcca

SEQ ID NO: 25 - RNA, 20 nt, synthetic construct
  source 1..20: mol_type = other RNA
gagcgactgggacacggtga

SEQ ID NO: 26 - RNA, 20 nt, synthetic construct
  source 1..20: mol_type = other RNA
gctgcgcaagaagggcccta

SEQ ID NO: 27 - RNA, 20 nt, synthetic construct
  source 1..20: mol_type = other RNA
ttgttctggccagcagcccc

SEQ ID NO: 28 - RNA, 20 nt, synthetic construct
  source 1..20: mol_type = other RNA
cttccagagccacatcatcg

SEQ ID NO: 29 - RNA, 20 nt, synthetic construct
  source 1..20: mol_type = other RNA
gggactcaccagagagaggt

SEQ ID NO: 30 - RNA, 20 nt, synthetic construct
  source 1..20: mol_type = other RNA
cggtcgaaatagaagcccta

SEQ ID NO: 31 - RNA, 20 nt, synthetic construct
  source 1..20: mol_type = other RNA
aaaaggatattgtgcaactg

SEQ ID NO: 32 - RNA, 20 nt, synthetic construct
  source 1..20: mol_type = other RNA
tgtgcatatttattacatcg

SEQ ID NO: 33 - RNA, 20 nt, synthetic construct
  source 1..20: mol_type = other RNA
tttgtgaagatcttgaccaa

SEQ ID NO: 34 - RNA, 20 nt, synthetic construct
  source 1..20: mol_type = other RNA
tgtcatgctgaaccgcattg

SEQ ID NO: 35 - RNA, 20 nt, synthetic construct
  source 1..20: mol_type = other RNA
ccactctatgaggatagtca

SEQ ID NO: 36 - RNA, 20 nt, synthetic construct
  source 1..20: mol_type = other RNA
ttgacatagaagaggcacaa

SEQ ID NO: 37 - RNA, 20 nt, synthetic construct
  source 1..20: mol_type = other RNA
gagtactacactcagcagca

SEQ ID NO: 38 - RNA, 20 nt, synthetic construct
  source 1..20: mol_type = other RNA
tcacgcacaagaaacgtcca

SEQ ID NO: 39 - RNA, 20 nt, synthetic construct
  source 1..20: mol_type = other RNA
aggtctcggtgaaaccacct

SEQ ID NO: 40 - RNA, 20 nt, synthetic construct
  source 1..20: mol_type = other RNA
agcattatccaaagagtccg

SEQ ID NO: 41 - RNA, 20 nt, synthetic construct
  source 1..20: mol_type = other RNA
atattaattcttaccagtgg

SEQ ID NO: 42 - RNA, 20 nt, synthetic construct
  source 1..20: mol_type = other RNA
agctttaaatcaaggttcat

SEQ ID NO: 43 - RNA, 20 nt, synthetic construct
  source 1..20: mol_type = other RNA
atcccgagccctaaggtgca

SEQ ID NO: 44 - RNA, 20 nt, synthetic construct
  source 1..20: mol_type = other RNA
ggcagcgcggaggacagcgt

SEQ ID NO: 45 - RNA, 20 nt, synthetic construct
  source 1..20: mol_type = other RNA
ctcagggggctactaccacc

SEQ ID NO: 46 - RNA, 20 nt, synthetic construct
  source 1..20: mol_type = other RNA
gtcaccgacgagaccagaag

SEQ ID NO: 47 - RNA, 20 nt, synthetic construct
  source 1..20: mol_type = other RNA
gtcgtggacttcgtactgct

SEQ ID NO: 48 - RNA, 20 nt, synthetic construct
  source 1..20: mol_type = other RNA
taatttttaggcaagtgtcg

SEQ ID NO: 49 - RNA, 20 nt, synthetic construct
  source 1..20: mol_type = other RNA
ttagctgttagacttgaata

SEQ ID NO: 50 - RNA, 20 nt, synthetic construct
  source 1..20: mol_type = other RNA
cgagagccgtcaacttgcgt

SEQ ID NO: 51 - RNA, 20 nt, synthetic construct
  source 1..20: mol_type = other RNA
cggcttcaactgcaaaggtg

SEQ ID NO: 52 - RNA, 20 nt, synthetic construct
  source 1..20: mol_type = other RNA
tatgaaaaagcagagcgact

SEQ ID NO: 53 - RNA, 20 nt, synthetic construct
  source 1..20: mol_type = other RNA
tctggcgggcgagctcacgc

SEQ ID NO: 54 - RNA, 20 nt, synthetic construct
  source 1..20: mol_type = other RNA
ctcacgctggttaccgccta

SEQ ID NO: 55 - RNA, 20 nt, synthetic construct
  source 1..20: mol_type = other RNA
aaagattacgaacttccctg

SEQ ID NO: 56 - RNA, 20 nt, synthetic construct
  source 1..20: mol_type = other RNA
gttaaaaacagacatgccta

SEQ ID NO: 57 - RNA, 20 nt, synthetic construct
  source 1..20: mol_type = other RNA
atgcctaaggaggttgtacc

SEQ ID NO: 58 - RNA, 20 nt, synthetic construct
  source 1..20: mol_type = other RNA
ctccaggtatcccatcgaaa

SEQ ID NO: 59 - RNA, 20 nt, synthetic construct
  source 1..20: mol_type = other RNA
caccaaatacgatagatcag

SEQ ID NO: 60 - RNA, 20 nt, synthetic construct
  source 1..20: mol_type = other RNA
tggcggcgtgaatggcaaga

SEQ ID NO: 61 - RNA, 20 nt, synthetic construct
  source 1..20: mol_type = other RNA
taggatggtagcacacaacc

SEQ ID NO: 62 - RNA, 20 nt, synthetic construct
  source 1..20: mol_type = other RNA
cagcagcagagccccgacgg

SEQ ID NO: 63 - RNA, 20 nt, synthetic construct
  source 1..20: mol_type = other RNA
cggcgtgcgaacggaatgtg

SEQ ID NO: 64 - RNA, 20 nt, synthetic construct
  source 1..20: mol_type = other RNA
tatagacgctgcccgacgtc

SEQ ID NO: 65 - RNA, 20 nt, synthetic construct
  source 1..20: mol_type = other RNA
tccaaagaagggtactgtgg

SEQ ID NO: 66 - RNA, 20 nt, synthetic construct
  source 1..20: mol_type = other RNA
acagtacccttctttggaat

SEQ ID NO: 67 - RNA, 20 nt, synthetic construct
  source 1..20: mol_type = other RNA
gcgacgggcgcatctacgtg

SEQ ID NO: 68 - RNA, 20 nt, synthetic construct
  source 1..20: mol_type = other RNA
cccgacctccataagtcctg

SEQ ID NO: 69 - RNA, 20 nt, synthetic construct
  source 1..20: mol_type = other RNA
ggggtcctcgaagcgcacga

SEQ ID NO: 70 - RNA, 20 nt, synthetic construct
  source 1..20: mol_type = other RNA
tgctctgtttagaagatgac

SEQ ID NO: 71 - RNA, 20 nt, synthetic construct
  source 1..20: mol_type = other RNA
atattcttttctagttaaag

SEQ ID NO: 72 - RNA, 20 nt, synthetic construct
  source 1..20: mol_type = other RNA
cctgtaaagaaacaaaagac

SEQ ID NO: 73 - RNA, 20 nt, synthetic construct
  source 1..20: mol_type = other RNA
tggagaaagacgtaacttcg

SEQ ID NO: 74 - RNA, 20 nt, synthetic construct
  source 1..20: mol_type = other RNA
tctgccctgaggtatgcgat

SEQ ID NO: 75 - RNA, 20 nt, synthetic construct
  source 1..20: mol_type = other RNA
attccgcttggtgaaaacga

SEQ ID NO: 76 - RNA, 20 nt, synthetic construct
  source 1..20: mol_type = other RNA
caggcacaatagaaacaacg

SEQ ID NO: 77 - RNA, 20 nt, synthetic construct
  source 1..20: mol_type = other RNA
ccatttgtaatgctgacttg

SEQ ID NO: 78 - RNA, 20 nt, synthetic construct
  source 1..20: mol_type = other RNA
ctgggtcacttgtgccgtgg

SEQ ID NO: 79 - RNA, 20 nt, synthetic construct
  source 1..20: mol_type = other RNA
gtcagggttctggatatctg

SEQ ID NO: 80 - RNA, 20 nt, synthetic construct
  source 1..20: mol_type = other RNA
tggatttagagtctctcagc

SEQ ID NO: 81 - RNA, 20 nt, synthetic construct
  source 1..20: mol_type = other RNA
ctgcggctgtggtccagctg

SEQ ID NO: 82 - RNA, 20 nt, synthetic construct
  source 1..20: mol_type = other RNA
acaaaactgtgctagacatg

SEQ ID NO: 83 - RNA, 20 nt, synthetic construct
  source 1..20: mol_type = other RNA
ttcttccccagcccaggtaa

SEQ ID NO: 84 - RNA, 20 nt, synthetic construct
  source 1..20: mol_type = other RNA
cgtcatgagcagattaaacc

SEQ ID NO: 85 - RNA, 20 nt, synthetic construct
  source 1..20: mol_type = other RNA
gagagcgcctgcgacccgag

SEQ ID NO: 86 - RNA, 20 nt, synthetic construct
  source 1..20: mol_type = other RNA
ccagcgggtgaagtacacca

SEQ ID NO: 87 - RNA, 20 nt, synthetic construct
  source 1..20: mol_type = other RNA
ggagcgcttttcgccgccag

SEQ ID NO: 88 - RNA, 20 nt, synthetic construct
  source 1..20: mol_type = other RNA
tgaggcctggaccttatgca

SEQ ID NO: 89 - RNA, 20 nt, synthetic construct
  source 1..20: mol_type = other RNA
cctggtggagtgaaccatga

SEQ ID NO: 90 - RNA, 20 nt, synthetic construct
  source 1..20: mol_type = other RNA
caagcacttaggttcccctg

SEQ ID NO: 91 - RNA, 20 nt, synthetic construct
  source 1..20: mol_type = other RNA
ggtctccctacaattcagcg

SEQ ID NO: 92 - RNA, 20 nt, synthetic construct
  source 1..20: mol_type = other RNA
cacagcgcgtgactgcaatg

SEQ ID NO: 93 - RNA, 20 nt, synthetic construct
  source 1..20: mol_type = other RNA
tctggggcaccaattctagg

SEQ ID NO: 94 - RNA, 20 nt, synthetic construct
  source 1..20: mol_type = other RNA
gagccatgcttggcttacga

SEQ ID NO: 95 - RNA, 20 nt, synthetic construct
  source 1..20: mol_type = other RNA
gtacaagtacttatctcatg

SEQ ID NO: 96 - RNA, 20 nt, synthetic construct
  source 1..20: mol_type = other RNA
gagataacaacataacaaca

SEQ ID NO: 97 - RNA, 20 nt, synthetic construct
  source 1..20: mol_type = other RNA
catattccatagtctttggg

SEQ ID NO: 98 - RNA, 20 nt, synthetic construct
  source 1..20: mol_type = other RNA
ctgccccttagcaacttagg

SEQ ID NO: 99 - RNA, 20 nt, synthetic construct
  source 1..20: mol_type = other RNA
tgtttaaaaatatgttgaca

SEQ ID NO: 100 - RNA, 20 nt, synthetic construct
  source 1..20: mol_type = other RNA
ccaggaatggaaactcacgc

SEQ ID NO: 101 - RNA, 20 nt, synthetic construct
  source 1..20: mol_type = other RNA
gaggccgctgaattaacccg

SEQ ID NO: 102 - RNA, 20 nt, synthetic construct
  source 1..20: mol_type = other RNA
atacacgcacacttgcagaa

SEQ ID NO: 103 - RNA, 20 nt, synthetic construct
  source 1..20: mol_type = other RNA
gagcagacagaaacccaggg

SEQ ID NO: 104 - RNA, 20 nt, synthetic construct
  source 1..20: mol_type = other RNA
tgagtctccaaacagaacag

SEQ ID NO: 105 - RNA, 20 nt, synthetic construct
  source 1..20: mol_type = other RNA
taatatcactgacttcacgg

SEQ ID NO: 106 - RNA, 20 nt, synthetic construct
  source 1..20: mol_type = other RNA
tacacacaatgtaagcagca

SEQ ID NO: 107 - RNA, 20 nt, synthetic construct
  source 1..20: mol_type = other RNA
gggagctcaattcgaaacca

SEQ ID NO: 108 - RNA, 20 nt, synthetic construct
  source 1..20: mol_type = other RNA
ttggacaggtgagacagtcg

SEQ ID NO: 109 - RNA, 20 nt, synthetic construct
  source 1..20: mol_type = other RNA
aagctcactcagatagtgtg

SEQ ID NO: 110 - RNA, 20 nt, synthetic construct
  source 1..20: mol_type = other RNA
caggagaaccaccttacacg

SEQ ID NO: 111 - RNA, 20 nt, synthetic construct
  source 1..20: mol_type = other RNA
ggacagaccctgattcacaa

SEQ ID NO: 112 - RNA, 20 nt, synthetic construct
  source 1..20: mol_type = other RNA
acatggcagtctatgaacag

SEQ ID NO: 113 - RNA, 20 nt, synthetic construct
  source 1..20: mol_type = other RNA
cctatagagagtactacttg

SEQ ID NO: 114 - RNA, 20 nt, synthetic construct
  source 1..20: mol_type = other RNA
ccaaccgggtcttcattacg

SEQ ID NO: 115 - RNA, 20 nt, synthetic construct
  source 1..20: mol_type = other RNA
tcaagcgtagagttccgagt

SEQ ID NO: 116 - RNA, 20 nt, synthetic construct
  source 1..20: mol_type = other RNA
tcatgcaattatggacccag

SEQ ID NO: 117 - RNA, 20 nt, synthetic construct
  source 1..20: mol_type = other RNA
cgggaaagtgactggccatg

SEQ ID NO: 118 - RNA, 20 nt, synthetic construct
  source 1..20: mol_type = other RNA
tgagattgaaatcaaatcgg

SEQ ID NO: 119 - RNA, 20 nt, synthetic construct
  source 1..20: mol_type = other RNA
tatgcaatattcatcacgcg

SEQ ID NO: 120 - RNA, 20 nt, synthetic construct
  source 1..20: mol_type = other RNA
aatgtgttaaatcaaatgca

SEQ ID NO: 121 - protein, 7 aa, synthetic construct
ENLYTQS

SEQ ID NO: 122 - protein, 5 aa, synthetic construct
DDDDK

SEQ ID NO: 123 - protein, 4 aa, synthetic construct
LVPR

SEQ ID NO: 124 - protein, 4 aa, synthetic construct
CPPC

SEQ ID NO: 125 - protein, 232 aa, Homo sapiens
MGSGPAAGPPLLLLGTPGSGKTALLFAAALEAAGEGQGPVLFLTRRPLQSMPRGTGTTLDPMRLQKIRFQYPPSTRELFRLLCSAHEAPGPAPSLLLLDGLEEYLAEDPEPQEAAYLIALLLDTAAHFSHRLGPGRDCGLMVALQTQEEAGSGDVLHLALLQRYFPAQCWLQPDAPGPGEHGLRACLEPGGLGPRTEWWVTFRSDGEMMIAPWPTQAGDPSSGKGSSSGGQP

SEQ ID NO: 126 - protein, 287 aa, Homo sapiens
MGSGQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 127 - protein, 205 aa, synthetic construct
MGSGSIFTDMIPAELLINEYKKGQSGAKHDNYVSVGRIMVAIYKNNSFKNTGTVKYQDSTHSGITMSKVFIDGKEYRIDIDTQHYEVQDFDTSGRQTTLILKRIDLYGGSGSGSPSPARPALRAPASATSGSRKRARPPAAPGRDQARPPARRRLRLSVDEVSSPSTPEAPDIPACPSPGQKIKKSTPAAGQPPHLTSAQDQDTI

SEQ ID NO: 128 - RNA, 103 nt, synthetic construct
  source 1..103: mol_type = other RNA
ggggccactagggacaggatgttttagagctagaaatagcaagttaaaataaggctagtccgttatcaacttgaaaaagtggcaccgagtcggtgcttttttt

SEQ ID NO: 129 - DNA, 20 bp, Homo sapiens
  source 1..20: mol_type = genomic DNA
ggggccactagggacaggat

(19) United States
(12) Patent Application Publication
(10) Pub. No.: US 2026/0308578 A1
(43) Pub. Date: Oct. 8, 2026

(54) NON-VIRAL CELL ENGINEERING

(71) Applicant: Arsenal Biosciences, Inc., South San Francisco, CA (US)

(72) Inventors: Jenessa Smith, Kalispell, MT (US); Robert Moot, San Francisco, CA (US); Alice Chang, Pacifica, CA (US); Robert Bao, San Francisco, CA (US); Brady Trevisan, South San Francisco, CA (US); Lina M. Leon, South San Francisco, CA (US); Annie Truong, South San Franciso, CA (US)

(21) Appl. No.: 19/417,202

(22) Filed: Dec. 11, 2025

Related U.S. Application Data
(63) Continuation of application No. PCT/US2024/034202, filed on Jun. 14, 2024.
(60) Provisional application No. 63/508,059, filed on Jun. 14, 2023.

(51) Int. Cl. C12N 15/11

(52) U.S. Cl. CPC C12N 15/11; C12N 2310/20

(57) ABSTRACT

Provided herein are compositions and methods for improving non-viral insertion of genes into safe harbor loci of cells such as immune cells.

### CROSS REFERENCE TO RELATED APPLICATIONS

This application is a continuation of International Application No.: PCT/US2024/034202 filed Jun. 14, 2024, which claims the benefit of U.S. Provisional Application No. 63/508,059, filed Jun. 14, 2023, which are hereby incorporated in its entirety by reference.

### SEQUENCE LISTING

The instant application contains a Sequence Listing which is hereby incorporated by reference in its entirety. Said XML copy, created on Jun. 10, 2024, is named ANB-220WO_SL.xml, and is 115,114 bytes in size.

### BACKGROUND

Cancer continues to present a significant clinical burden despite the substantial research efforts and scientific advances in cancer therapies. Blood and bone marrow cancers are frequently diagnosed cancer types, including multiple myelomas, leukemia, and lymphomas. Current treatment options for these cancers are not effective for all patients and/or can have substantial adverse side effects. Other types of cancer also remain challenging to treat using existing therapeutic options. Cancer immunotherapies are a promising solution because they can be highly specific, allowing for increased therapeutic effectiveness and the mitigation of side effects.

Genetically engineered immune cell therapy is a growing field with promising applications for the treatment of diseases including, but not limited to, cancer. Through the alteration of coding and/or non-coding genomic regions, researchers are identifying transgenes and insertion sites within cells that facilitate, for example, enhanced cell function, arrest cell growth, induced cell death, and tumor size/volume reduction. The identification of safe harbor sites (SHS) has improved outcomes of genome-engineering therapies.

However, viral insertion of therapeutic genes into immune cells can lead to unanticipated off target insertions and poor gene transduction and editing efficiency. In addition, the viral vectors pose challenges to high throughput manufacturing processes. Non-viral gene editing methods can address these problems but can also have lower gene transfer efficiencies and total edited cell yields. Thus, additional methods to improve non-viral gene editing are required.

### SUMMARY

In one aspect, provided herein are compositions comprising a solution comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In one aspect, provided herein are compositions comprising a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In one aspect, provided herein are compositions comprising a solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In one aspect, provided herein are compositions comprising a solution comprising a histone deacetylase (HDAC) inhibitor comprising at least one of sodium phenylbutyrate, quisinostat, or panobinostat, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In one aspect, provided herein are compositions comprising a solution comprising a ribonucleoprotein complex (RNP), a DNA template, and at least one of an RNase inhibitor, N acetyl-L-cysteine (NAC), a histone deacetylase (HDAC) inhibitor, and/or an osmolarity-adjusting agent, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In some embodiments, the solution comprises at least:
i. the N acetyl-L-cysteine (NAC) and the osmolarity-adjusting agent;
ii. the RNase inhibitor and the N acetyl-L-cysteine (NAC);
iii, the RNase inhibitor and the osmolarity-adjusting agent;
iv. the N acetyl-L-cysteine (NAC) and the histone deacetylase (HDAC) inhibitor;
v. the osmolarity-adjusting agent and the histone deacetylase (HDAC) inhibitor;
vi. the RNase-inhibitor and the histone deacetylase (HDAC) inhibitor;
vii, the RNase inhibitor, the N acetyl-L-cysteine (NAC), and the histone deacetylase (HDAC) inhibitor;
viii, the N acetyl-L-cysteine (NAC), the osmolarity-adjusting agent, and the histone deacetylase (HDAC) inhibitor;
ix. the RNase inhibitor, the osmolarity-adjusting agent, and the histone deacetylase (HDAC) inhibitor;
x. the RNase inhibitor, the N acetyl-L-cysteine (NAC), and the osmolarity-adjusting agent; or
xi. the RNase inhibitor, the N acetyl-L-cysteine (NAC), the osmolarity-adjusting agent, and the histone deacetylase (HDAC) inhibitor.

In some embodiments, the RNase inhibitor is present in the solution at a final concentration of between about 0.5 to 2 U/μl.

In some embodiments, the RNase inhibitor is present in the solution at a final concentration of about 1 U/μL.

In some embodiments, the RNase inhibitor is an RNase A, B, C, T1, or T2 inhibitor.

In some embodiments, the RNase inhibitor is a murine, rat, or human RNase inhibitor.

In some embodiments, the N acetyl-L-cysteine (NAC) is present in the solution at a final concentration of between about 1-10 mM.

In some embodiments, the N acetyl-L-cysteine (NAC) is present in the solution at a final concentration of about 2.5 mM.

In some embodiments, the osmolarity-adjusting agent is sorbitol, glycerol, or glycine.

In some embodiments, the osmolarity-adjusting agent is sorbitol.

In some embodiments, the sorbitol is present in the solution at a final concentration of between about 100-400 mM.

In some embodiments, the sorbitol is present in the solution at a final concentration of about 190 mM or 200 mM.

In some embodiments, the osmolarity-adjusting agent is glycerol.

In some embodiments, the glycerol is present in the solution at a final concentration of between about 100-400 mM.

In some embodiments, the glycerol is present in the solution at a final concentration of about 170 mM.

In one aspect, provided herein are compositions comprising a solution comprising a cell comprising at least one heterologous DNA template inserted into a target region of the genome and a histone deacetylase (HDAC) inhibitor comprising at least one of sodium phenylbutyrate, quisinostat, or Panobinostat.

In some embodiments, the histone deacetylase (HDAC) inhibitor is an HDAC1 or HDAC2 inhibitor.

In some embodiments, the HDAC inhibitor is sodium phenylbutyrate, quisinostat, Panobinostat, phenylbutyric acid, curcumin, mocetinostat, romidespin, SIS17, splitomicin, trichostatin-A, tucidinostat, etinostat, sodium butyrate, valproic acid, CXD101, KT-531, ITF3756, tubastatin A, vorinostat, BML-210, belinostat, Abexinostat, dacinostat, CUDC-101, droxinostat, MC1568, pracinostat, divalproex sodium, PCI-3405, SR-4370, givinostat, tubacin, AR-42, (−)-parthenolide, resminostat, fimepinostat, M344, tacedinaline, sinapinic acid, biphenyl-4-sufonyl chloride, sulforaphane, UF010, suberohdroxamic acid, NKL 22, TC-H 106, RGFP966, HPOB, RG2833, TMP269, nexturastat A, domatinostat, LMK-235, santacruzamate A, CAY10603, tasquinimod, BG45, BRD73954, ricolinostat, scriptaid, citarinostat, WT161, TMP195, ACY-738, SKLB-23bb, tinostamustine, TH34, BRD3308, raddeanin A, isoguanosine, KA2507, ITSA-1, or an RNA interference (RNAi) molecule.

In some embodiments, the HDAC inhibitor is sodium phenylbutyrate, quisinostat or panobinostat.

In some embodiments, the sodium phenylbutyrate is present in the solution at a final concentration of between about 15.6 μM-4 mM.

In some embodiments, the sodium phenylbutyrate is present in the solution at a final concentration of about 1 mM, optionally wherein the solution comprises 1% DMSO.

In some embodiments, the quisinostat is present in the solution at a final concentration of between about 8 nM-200 nM.

In some embodiments, the quisinostat is present in the solution at a final concentration of about 16 nM.

In some embodiments, the panobinostat is present in the solution at a final concentration of between about 3 nM-1 μM.

In some embodiments, the panobinostat is present in the solution at a final concentration of about 37.5 nM.

In some embodiments, the solution comprises at least one of a final concentration of about 0.5 to 2 U/μl RNase inhibitor, a final concentration of about 1-10 mM NAC, a final concentration of about 100-400 mM sorbitol, and/or a final concentration of about 15.6 μM-4 mM sodium phenylbutyrate, 8 nM-200 nM quisinostat, or 3 nM-1 μM panobinostat.

In some embodiments, the solution comprises at least one of a final concentration of about 1 U/μl RNase inhibitor, a final concentration of about 2.5 mM NAC, a final concentration of about 200 mM sorbitol, and/or a final concentration of about 1 mM sodium phenylbutyrate, 0.016 μM quisinostat, or 0.0375 μM panobinostat.

In some embodiments, the nuclease domain comprises a CRISPR-associated endonuclease (Cas), optionally a Cas9 nuclease.

In some embodiments, the size of the DNA template is greater than or equal to 300 nucleotides.

In some embodiments, the size of the DNA template is greater than or equal to about 0.3 kb, 0.5 kb, 1.0 kb, 1.5 kb, 2.0 kb, 2.5 kb, 3.0 kb, 3.5 kb, 4.0 kb, 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb, 10.1 kb, 10.2 kb, 10.3 kb, 10.4 kb, 10.5 kb, 10.6 kb, 10.7 kb, 10.8 kb, 10.9 kb, 11.0 kb, 11.1 kb, 11.2 kb, 11.3 kb, 11.4 kb, 11.5 kb, 11.6 kb, 11.7 kb, 11.8 kb, 11.9 kb, 12.0 kb, 12.1 kb, 12.2 kb, 12.3 kb, 12.4 kb, 12.5 kb, 12.6 kb, 12.7 kb, 12.8 kb, 12.9 kb, 13.0 kb, or larger, or any size DNA template in between these sizes.

In some embodiments, the size of the DNA template is about 0.3 kb to about 13 kb, about 0.3 kb to about 0.5 kb, about 0.3 kb to about 1 kb, about 0.3 kb to about 4 kb, about 0.3 kb to about 3 kb, about 0.3 kb to about 5 kb, about 0.3 kb to about 7 kb, about 0.3 kb to about 10 kb, about 0.5 kb to about 1 kb, about 0.5 kb to about 3 kb, about 0.5 kb to about 5 kb, about 0.5 kb to about 7 kb, about 0.5 kb to about 10 kb, about 0.5 kb to about 13 kb, about 1 kb to about 3 kb, about 1 kb to about 5 kb, about 1 kb to about 7 kb, about 1 kb to about 10 kb, about 1 kb to about 13 kb, about 5 kb to about 13 kb, about 5 kb to about 9 kb, about 5 kb to about 8 kb, about 5 kb to about 7 kb, about 5 kb to about 6 kb, about kb 6 to about 13 kb, about kb, 6 to about 10 kb, about 6 kb to about 9 kb, about 6 kb to about 8 kb, about 6 kb to about 7 kb, about 7 kb to about 13 kb, about 7 kb to about 10 kb, about 7 kb to about 9 kb, about 7 kb to about 8 kb, about 8 kb to about 13 kb, about 8 kb to about 10 kb, about 8 kb to about 9 kb, about 9 kb to about 13 kb, about 9 kb to about 10 kb, about 10 kb to about 13 kb, or about 11 kb to about 13 kb.

In some embodiments, the composition comprises a cell comprising the genomic sequences flanking the insertion site in the genome of the cell.

In some embodiments, the cell is a mammalian cell, a human cell, a hematopoietic cell, an immune cell, a primary immune cell, or a primary human immune cell.

In some embodiments, the cell is a primary human immune cell.

In some embodiments, the immune cell is a natural killer (NK) cell, a T cell, a CD8+ T cell, a CD4+ T cell, a primary T cell, or a T cell progenitor.

In some embodiments, the immune cell is a primary T cell.

In some embodiments, the immune cell is a primary human T cell.

In some embodiments, the immune cell is undifferentiated.

In some embodiments, the immune cell is CD45RA⁺ and CCR7⁺, CD45RA⁺ and CCR7⁻, CD45RA⁻ and CCR7⁻, or CD45RA⁻ and CCR7⁺.

In some embodiments, the cell is virus-free.

In some embodiments, the cell comprises an exogenous homologous recombination or DNA repair modulation protein.

In some embodiments, the exogenous homologous recombination or DNA repair modulation protein is encoded on an episomal plasmid or an mRNA molecule.

In some embodiments, the exogenous homologous recombination or DNA repair modulation protein is SWSAP1, a dominant negative KU80, or an AcrIIA8-CDT1 fusion protein.

In some embodiments, the SWSAP1 protein comprises the sequence as set forth in SEQ ID NO: 125; the dominant negative KU80 protein comprises the sequence as set forth in SEQ ID NO: 126, or the AcrIIA8-CDT1 fusion protein comprises the sequence as set forth in SEQ ID NO: 127.

In some embodiments, comprising obtaining the cell from a patient and introducing the DNA template in vitro or ex vivo.

In some embodiments, the target region of the genome of the cell is a T Cell Receptor Alpha Constant (TRAC) locus or a genomic safe harbor (GSH).

In some embodiments, the safe harbor locus is selected from any one of the integration sites designated: GS94, GS88, GS89, GS90, GS91, GS92, GS93, GS95, GS96, GS97, GS98, GS99, GS100, GS101, GS102, GS103, GS104, GS105, GS106, GS107, GS108, GS109, GS110, GS111, GS112, GS113, GS114, GS115, GS116, GS117, GS118, GS119, or GS120.

In some embodiments, the safe harbor locus is the GS94 integration site.

In some embodiments, the safe harbor locus is selected from: chr11:128340000-128350000, chr10:33130000-33140000, chr10:72290000-72300000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, or chr9:7970000-7980000.

In some embodiments, the safe harbor locus is a gene selected from: APRT, B2M, CAPNS1, CBLB, CD2, CD3E, CD3G, CD5, EDF1, FTL, PTEN, PTPN2, PTPN6, PTPRC, PTPRCAP, RPS23, RTRAF, SERF2, SLC38A1, SMAD2, SOCS1, SRP14, SRSF9, SUB1, TET2, TIGIT, TRAC, or TRIM28.

In some embodiments, comprising one or more gRNAs comprising any one of SEQ ID NOS: 1-120.

In some embodiments, the cell is CD45RA⁺ and CCR7⁺ after insertion of the at least one sequence into the safe harbor locus.

In some embodiments, the DNA template is a double-stranded DNA template or a single-stranded DNA template.

In some embodiments, the DNA template is a linear DNA template or a circular DNA template, optionally wherein the circular DNA template is a plasmid.

In some embodiments, the DNA template comprises a heterologous sequence.

In some embodiments, the DNA template comprises a gene.

In some embodiments, the DNA template comprises a priming receptor comprising a transcription factor.

In some embodiments, the DNA template comprises a chimeric antigen receptor (CAR).

In some embodiments, the DNA template comprises a chimeric antigen receptor (CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction:
i. the inducible promoter;
ii. the chimeric antigen receptor;
iii, the constitutive promoter; and
iv. the priming receptor.

In some embodiments, the DNA template comprises, in a 5′ to 3′ direction:
i. the constitutive promoter;
ii. the priming receptor;
iii, the inducible promoter; and
iv. the chimeric antigen receptor.

In some embodiments, the priming receptor comprises, in an N terminus to C terminus direction:
i. an extracellular antigen-binding domain having a binding affinity for an antigen;
ii. a transmembrane domain comprising one or more ligand-inducible proteolytic cleavage sites; and
iii. an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) or stop transfer sequence (STS) positioned between the transmembrane domain and the intracellular domain.

In some embodiments, the transcription factor binds to the inducible promoter and induces expression of the CAR.

In some embodiments, the CAR comprises, from N-terminus to C-terminus,
i. an extracellular antigen-binding domain having a binding affinity for an antigen;
ii. a transmembrane domain;
iii. an intracellular co-stimulatory domain; and
iv. an intracellular activation domain.

In some embodiments, the priming receptor and the CAR bind different antigens.

In some embodiments, the priming receptor and the CAR bind the same antigen.

In one aspect, provided herein are polypeptides comprising an AcrIIA8 peptide fused to a CDT1 peptide.

In some embodiments, the polypeptide comprises the sequence as set forth in SEQ ID NO: 127.

In one aspect, provided herein are primary immune cells comprising an exogenous homologous recombination or DNA repair modulation protein.

In some embodiments, the exogenous homologous recombination or DNA repair modulation protein is encoded on an episomal plasmid or an mRNA molecule.

In some embodiments, the exogenous homologous recombination or DNA repair modulation protein is SWSAP1, a dominant negative KU80, or an AcrIIA8-CDT1 fusion protein.

In some embodiments, the SWSAP1 protein comprises the sequence as set forth in SEQ ID NO: 125; the dominant negative KU80 protein comprises the sequence as set forth in SEQ ID NO: 126, or the AcrIIA8-CDT1 fusion protein comprises the sequence as set forth in SEQ ID NO: 127.

In some embodiments, the cell is a human cell, a hematopoietic cell, or a primary human immune cell.

In some embodiments, the immune cell is a natural killer (NK) cell, a T cell, a CD8+ T cell, a CD4+ T cell, a primary T cell, or a T cell progenitor.

In some embodiments, the immune cell is a primary T cell.

In some embodiments, the immune cell is a primary human T cell.

In some embodiments, the immune cell is undifferentiated.

In some embodiments, the immune cell is CD45RA⁺ and CCR7⁺, CD45RA⁺ and CCR7⁻, CD45RA⁻ and CCR7⁻, or CD45RA⁻ and CCR7⁺.

In some embodiments, the cell is virus-free.

In some embodiments, the cell comprises a DNA template wherein the 5′ and 3′ ends of the DNA template comprise nucleotide sequences that are homologous to genomic sequences flanking an insertion site in the genome of the cell.

In some embodiments, the target region of the genome of the cell is a T Cell Receptor Alpha Constant (TRAC) locus or a genomic safe harbor (GSH).

In some embodiments, the safe harbor locus is selected from any one of the integration sites designated: GS94, GS88, GS89, GS90, GS91, GS92, GS93, GS95, GS96, GS97, GS98, GS99, GS100, GS101, GS102, GS103, GS104, GS105, GS106, GS107, GS108, GS109, GS110, GS111, GS112, GS113, GS114, GS115, GS116, GS117, GS118, GS119, or GS120.

In some embodiments, the safe harbor locus is the GS94 integration site.

In some embodiments, the safe harbor locus is selected from: chr11:128340000-128350000, chr10:33130000-33140000, chr10:72290000-72300000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, or chr9:7970000-7980000.

In some embodiments, the safe harbor locus is a gene selected from: APRT, B2M, CAPNS1, CBLB, CD2, CD3E, CD3G, CD5, EDF1, FTL, PTEN, PTPN2, PTPN6, PTPRC, PTPRCAP, RPS23, RTRAF, SERF2, SLC38A1, SMAD2, SOCS1, SRP14, SRSF9, SUB1, TET2, TIGIT, TRAC, or TRIM28.

In some embodiments, one or more gRNAs comprising any one of SEQ ID NOS: 1-120.

In some embodiments, the cell is CD45RA⁺ and CCR7⁺ after insertion of the at least one sequence into the safe harbor locus.

In some embodiments, the DNA template is a double-stranded DNA template or a single-stranded DNA template.

In some embodiments, the DNA template is a linear DNA template or a circular DNA template, optionally wherein the circular DNA template is a plasmid.

In some embodiments, the DNA template comprises a heterologous sequence.

In some embodiments, the DNA template comprises a gene.

In some embodiments, the DNA template comprises a priming receptor comprising a transcription factor, a chimeric antigen receptor (CAR), or a chimeric antigen receptor (CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction:
i. the inducible promoter;
ii. the chimeric antigen receptor;
iii, the constitutive promoter; and
iv. the priming receptor.

In some embodiments, the DNA template comprises, in a 5′ to 3′ direction:
i. the constitutive promoter;
ii. the priming receptor;
iii, the inducible promoter; and
iv. the chimeric antigen receptor.

In some embodiments, the priming receptor comprises, in an N terminus to C terminus direction:
i. an extracellular antigen-binding domain having a binding affinity for an antigen;
ii. a transmembrane domain comprising one or more ligand-inducible proteolytic cleavage sites; and
iii. an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) or stop transfer sequence (STS) positioned between the transmembrane domain and the intracellular domain.

In some embodiments, the transcription factor binds to the inducible promoter and induces expression of the CAR.

In some embodiments, the CAR comprises, from N-terminus to C-terminus,
i. an extracellular antigen-binding domain having a binding affinity for an antigen;
ii. a transmembrane domain;
iii. an intracellular co-stimulatory domain; and
iv. an intracellular activation domain.

In some embodiments, the priming receptor and the CAR bind different antigens.

In some embodiments, the priming receptor and the CAR bind the same antigen.

In one aspect, provided herein are methods of editing a cell, comprising:
i. providing a cell comprising an exogenous homologous recombination or DNA repair modulation protein;
ii. providing a solution comprising a ribonucleoprotein complex (RNP), a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
iii. editing the cell via insertion of the DNA template into the insertion site in the genome of the cell.

In one aspect, provided herein are methods of editing a cell, comprising:
i. providing solution comprising a histone deacetylase (HDAC) inhibitor comprising at least one of sodium phenylbutyrate, quisinostat, or panobinostat and a cell;
ii. contacting the cell with a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
iii. editing the cell via insertion of the DNA template into the insertion site in the genome of the cell.

In some embodiments, the method comprises contacting the edited cell with a solution comprising a histone deacetylase (HDAC) inhibitor comprising at least one of sodium phenylbutyrate, quisinostat, or Panobinostat.

In one aspect, provided herein are methods of editing a cell, comprising:
i. providing a solution comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
ii. editing the cell via insertion of the DNA template into the insertion site in the genome of the cell.

In one aspect, provided herein are methods of editing a cell, comprising:
i. providing a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
ii. editing the cell via insertion of the DNA template into the insertion site in the genome of the cell.

In one aspect, provided herein are methods of editing a cell, comprising:
i. providing solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
ii. editing the cell via insertion of the DNA template into the insertion site in the genome of the cell.

In one aspect, provided herein are methods of editing a cell, comprising:
i. providing solution comprising a histone deacetylase (HDAC) inhibitor comprising at least one of sodium phenylbutyrate, quisinostat, or panobinostat, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
ii. editing the cell via insertion of the DNA template into the insertion site in the genome of the cell.

In one aspect, provided herein are methods of editing a cell, comprising:
i. providing a cell comprising an exogenous homologous recombination or DNA repair modulation protein;
ii. providing a solution comprising a ribonucleoprotein complex (RNP), a DNA template, and at least one of an RNase inhibitor, N acetyl-L-cysteine (NAC), an osmolarity-adjusting agent or a histone deacetylase (HDAC) inhibitor, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
iii. editing the cell via insertion of the DNA template into the insertion site in the genome of the cell.

In one aspect, provided herein are methods of editing a cell, comprising:
i. providing a solution comprising a ribonucleoprotein complex (RNP), a DNA template, and at least one of an RNase inhibitor, N acetyl-L-cysteine (NAC), an osmolarity-adjusting agent or a histone deacetylase (HDAC) inhibitor, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
ii. editing the cell via insertion of the DNA template into the insertion site in the genome of the cell.

In some embodiments, the solution comprises at least:
i. the N acetyl-L-cysteine (NAC) and the osmolarity-adjusting agent;
ii. the RNase inhibitor and the N acetyl-L-cysteine (NAC);
iii, the RNase inhibitor and the osmolarity-adjusting agent;
iv. the N acetyl-L-cysteine (NAC) and the histone deacetylase (HDAC) inhibitor;
v. the osmolarity-adjusting agent and the histone deacetylase (HDAC) inhibitor;
vi. the RNase-inhibitor and the histone deacetylase (HDAC) inhibitor;
vii, the RNase inhibitor, the N acetyl-L-cysteine (NAC), and the histone deacetylase (HDAC) inhibitor;
viii, the N acetyl-L-cysteine (NAC), the osmolarity-adjusting agent, and the histone deacetylase (HDAC) inhibitor;
ix. the RNase inhibitor, the osmolarity-adjusting agent, and the histone deacetylase (HDAC) inhibitor;
x. the RNase inhibitor, the N acetyl-L-cysteine (NAC), and the osmolarity-adjusting agent or
xi. the RNase inhibitor, the N acetyl-L-cysteine (NAC), the osmolarity-adjusting agent, and the histone deacetylase (HDAC) inhibitor.

In some embodiments, the RNase inhibitor is present in the solution at a final concentration of between about 0.5-2 U/μL.

In some embodiments, the RNase inhibitor is present in the solution at a final concentration of about 1 U/μL.

In some embodiments, the RNase inhibitor is an RNase A, B, C, T1, or T2 inhibitor.

In some embodiments, the RNase inhibitor is a murine, rat, or human RNase inhibitor.

In some embodiments, the N acetyl-L-cysteine (NAC) is present in the solution at a final concentration of between about 1-10 mM.

In some embodiments, the N acetyl-L-cysteine (NAC) is present in the solution at a final concentration of about 2.5 mM.

In some embodiments, the osmolarity-adjusting agent is sorbitol, glycerol, or glycine.

In some embodiments, the osmolarity-adjusting agent is sorbitol.

In some embodiments, the sorbitol is present in the solution at a final concentration of between about 100-400 mM.

In some embodiments, the sorbitol is present in the solution at a final concentration of about 190 mM or 200 mM.

In some embodiments, the wherein the osmolarity-adjusting agent is glycerol.

In some embodiments, the glycerol is present in the solution at a final concentration of between about 100-400 mM.

In some embodiments, the glycerol is present in the solution at a final concentration of about 170 mM.

In some embodiments, the histone deacetylase (HDAC) inhibitor is an HDAC1 or HDAC2 inhibitor.

In some embodiments, the HDAC inhibitor is sodium phenylbutyrate, quisinostat, Panobinostat, phenylbutyric acid, curcumin, mocetinostat, romidespin, SIS17, splitomicin, trichostatin-A, tucidinostat, etinostat, sodium butyrate, valproic acid, CXD101, KT-531, ITF3756, tubastatin A, vorinostat, BML-210, belinostat, Abexinostat, dacinostat, CUDC-101, droxinostat, MC1568, pracinostat, divalproex sodium, PCI-3405, SR-4370, givinostat, tubacin, AR-42, (−)-parthenolide, resminostat, fimepinostat, M344, tacedinaline, sinapinic acid, biphenyl-4-sufonyl chloride, sulforaphane, UF010, suberohdroxamic acid, NKL 22, TC-H 106, RGFP966, HPOB, RG2833, TMP269, nexturastat A, domatinostat, LMK-235, santacruzamate A, CAY10603, tasquinimod, BG45, BRD73954, ricolinostat, scriptaid, citarinostat, WT161, TMP195, ACY-738, SKLB-23bb, tinostamustine, TH34, BRD3308, raddeanin A, isoguanosine, KA2507, or ITSA-1.

In some embodiments, the HDAC inhibitor is sodium phenylbutyrate, quisinostat or Panobinostat.

In some embodiments, the sodium phenylbutyrate is present in the solution at a final concentration of between about 15.6 μM-4 mM.

In some embodiments, the sodium phenylbutyrate is present in the solution at a final concentration of about 1 mM, optionally wherein the solution comprises 1% DMSO.

In some embodiments, the quisinostat is present in the solution at a final concentration of between about 8 nM-200 nM.

In some embodiments, the Quisinostat is present in the solution at a final concentration of about 16 nM.

In some embodiments, the panobinostat is present in the solution at a final concentration of between about 3 nM-1 μM.

In some embodiments, the Panobinostat is present in the solution at a final concentration of about 37.5 nM.

In some embodiments, the solution comprises at least one of a final concentration of about 0.5-2 U/μL RNase inhibitor, a final concentration of about 1-10 mM NAC, a final concentration of about 100-400 mM sorbitol and/or a final concentration of about 15.6 μM-4 mM sodium phenylbutyrate, 8 nM-200 nM quisinostat, or 3 nM-1 μM panobinostat.

In some embodiments, the solution comprises at least one of a final concentration of about 1 U/μL RNase inhibitor, a final concentration of about 2.5 mM NAC, a final concentration of about 200 mM sorbitol, and/or a final concentration of about 1 mM sodium phenylbutyrate, 0.016 μM quisinostat, or 0.0375 μM panobinostat.

In some embodiments, the exogenous homologous recombination or DNA repair modulation protein is encoded on an episomal plasmid or an mRNA molecule.

In some embodiments, the exogenous homologous recombination or DNA repair modulation protein is SWSAP1, a dominant negative KU80, or an AcrIIA8-CDT1 fusion protein.

In some embodiments, the SWSAP1 protein comprises the sequence as set forth in SEQ ID NO: 125; the dominant negative KU80 protein comprises the sequence as set forth in SEQ ID NO: 126, or the AcrIIA8-CDT1 fusion protein comprises the sequence as set forth in SEQ ID NO: 127

In some embodiments, method further comprising non-virally introducing the RNP complex and DNA template into the cell, wherein the guide RNA specifically hybridizes to a target region of the genome of the cell, and wherein the nuclease domain cleaves the target region to create the insertion site in the genome of the cell.

In some embodiments, non-virally introducing comprises electroporation.

In some embodiments, the electroporation comprises at least one cycle comprising at least one electrical pulse.

In some embodiments, the at least one cycle comprises at least five or more electrical pulses.

In some embodiments, the electrical pulse is about 2300 volts.

In some embodiments, the electrical pulse has a duration of about 3.0 ms.

In some embodiments, the cycle has a pulse interval of 500 ms.

In some embodiments, the electroporation comprises at least one cycle carried out using a setting of: 2300 volts, 3.0 ms pulse duration, five pulses, and 500 ms pulse interval.

In some embodiments, the exogenous homologous recombination or DNA repair modulation protein increases insertion of the DNA template into the genome of the cell as compared to a cell that does not comprise the exogenous homologous recombination or DNA repair modulation protein.

In some embodiments, the solution comprising at least one of an RNase inhibitor, N acetyl-L-cysteine (NAC), an osmolarity-adjusting agent, or a histone deacetylase (HDAC) inhibitor increases insertion of the DNA template into the genome of the cell as compared to a control solution that does not comprise the at least one of an RNase inhibitor, N acetyl-L-cysteine (NAC), an osmolarity-adjusting agent, or a histone deacetylase (HDAC) inhibitor.

In some embodiments, the method comprises incubating the cell with a HDAC inhibitor solution for about 2 days prior to non-virally introducing the RNP complex and DNA template into the cell.

In some embodiments, the HDAC inhibitor solution comprises a final concentration of about 0.1 mM sodium phenylbutyrate.

In some embodiments, the insertion of the DNA template into the genome of the cell is increased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution.

In some embodiments, the insertion of the DNA template into the genome of the cell is increased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution.

In some embodiments, the solution increases expansion of the edited cell as compared to a control solution that does not comprise the at least one of an RNase inhibitor, N acetyl-L-cysteine (NAC), an osmolarity-adjusting agent, or a histone deacetylase (HDAC) inhibitor.

In some embodiments, the expansion of the edited cell is increased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution.

In some embodiments, the expansion of the edited cell is increased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution.

In some embodiments, the solution increases yield of an edited cell as compared to a control solution that does not comprise the at least one of an RNase inhibitor, N acetyl-L-cysteine (NAC), an osmolarity-adjusting agent, or a histone deacetylase (HDAC) inhibitor.

In some embodiments, the yield of the edited cell is increased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution.

In some embodiments, the yield of the edited cell is increased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, 4-fold, 4-fold, 4.25-fold, 4.5-fold, 4.75-fold, 5-fold, 5.25-fold, 5.5-fold, 5.75-fold, 6-fold, 6.25-fold, 6.5-fold, 6.75-fold, or more, as compared to the control solution.

In some embodiments, the solution comprising at least one of an RNase inhibitor, N acetyl-L-cysteine (NAC) or an osmolarity-adjusting agent decreases death of the cell during the non-viral introduction of the RNP complex and DNA template into the cell as compared to a control solution that does not comprise the at least one of an RNase inhibitor, N acetyl-L-cysteine (NAC), an osmolarity-adjusting agent, or a histone deacetylase (HDAC) inhibitor.

In some embodiments, the death of the cell is decreased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution.

In some embodiments, the death of the cell is decreased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution.

In some embodiments, the nuclease domain comprises a CRISPR-associated endonuclease (Cas), optionally a Cas9 nuclease.

In some embodiments, the size of the DNA template is greater than or equal to 5 kilobase nucleotides.

In some embodiments, the size of the DNA template is greater than or equal to about 0.3 kb, 0.5 kb, 1.0 kb, 1.5 kb, 2.0 kb, 2.5 kb, 3.0 kb, 3.5 kb, 4.0 kb, 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb, 10.1 kb, 10.2 kb, 10.3 kb, 10.4 kb, 10.5 kb, 10.6 kb, 10.7 kb, 10.8 kb, 10.9 kb, 11.0 kb, 11.1 kb, 11.2 kb, 11.3 kb, 11.4 kb, 11.5 kb, 11.6 kb, 11.7 kb, 11.8 kb, 11.9 kb, 12.0 kb, 12.1 kb, 12.2 kb, 12.3 kb, 12.4 kb, 12.5 kb, 12.6 kb, 12.7 kb, 12.8 kb, 12.9 kb, 13.0 kb, or larger, or any size DNA template in between these sizes.

In some embodiments, the size of the DNA template is about 0.3 kb to about 13 kb, about 0.3 kb to about 0.5 kb, about 0.3 kb to about 1 kb, about 0.3 kb to about 4 kb, about 0.3 kb to about 3 kb, about 0.3 kb to about 5 kb, about 0.3 kb to about 7 kb, about 0.3 kb to about 10 kb, about 0.5 kb to about 1 kb, about 0.5 kb to about 3 kb, about 0.5 kb to about 5 kb, about 0.5 kb to about 7 kb, about 0.5 kb to about 10 kb, about 0.5 kb to about 13 kb, about 1 kb to about 3 kb, about 1 kb to about 5 kb, about 1 kb to about 7 kb, about 1 kb to about 10 kb, about 1 kb to about 13 kb, about 5 kb to about 13 kb, about 5 kb to about 9 kb, about 5 kb to about 8 kb, about 5 kb to about 7 kb, about 5 kb to about 6 kb, about kb 6 to about 13 kb, about kb, 6 to about 10 kb, about 6 kb to about 9 kb, about 6 kb to about 8 kb, about 6 kb to about 7 kb, about 7 kb to about 13 kb, about 7 kb to about 10 kb, about 7 kb to about 9 kb, about 7 kb to about 8 kb, about 8 kb to about 13 kb, about 8 kb to about 10 kb, about 8 kb to about 9 kb, about 9 kb to about 13 kb, about 9 kb to about 10 kb, about 10 kb to about 13 kb, or about 11 kb to about 13 kb.

In some embodiments, the cell is a mammalian cell, a human cell, a hematopoietic cell, an immune cell, a primary immune cell, or a primary human immune cell.

In some embodiments, the cell is a primary human immune cell.

In some embodiments, the immune cell is a natural killer (NK) cell, a T cell, a CD8+ T cell, a CD4+ T cell, a primary T cell, or a T cell progenitor cell.

In some embodiments, the immune cell is a primary T cell.

In some embodiments, the immune cell is a primary human T cell.

In some embodiments, the immune cell is undifferentiated.

In some embodiments, the immune cell is CD45RA⁺ and CCR7⁺, CD45RA⁺ and CCR7⁻, CD45RA⁻ and CCR7⁻, or CD45RA⁻ and CCR7⁺.

In some embodiments, the cell is virus-free.

In some embodiments, comprising obtaining the cell from a patient and introducing the DNA template in vitro or ex vivo.

In some embodiments, the target region of the genome of the cell is a T Cell Receptor Alpha Constant (TRAC) locus or a genomic safe harbor (GSH).

In some embodiments, the safe harbor locus is selected from any one of the integration sites designated: GS94, GS88, GS89, GS90, GS91, GS92, GS93, GS95, GS96, GS97, GS98, GS99, GS100, GS101, GS102, GS103, GS104, GS105, GS106, GS107, GS108, GS109, GS110, GS111, GS112, GS113, GS114, GS115, GS116, GS117, GS118, GS119, or GS120.

In some embodiments, the safe harbor locus is the GS94 integration site.

In some embodiments, the sgRNA target locus is selected from: chr11:128340000-128350000, chr10:33130000-33140000, chr10:72290000-72300000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, or chr9:7970000-7980000.

In some embodiments, the sgRNA target locus is a gene selected from: APRT, B2M, CAPNS1, CBLB, CD2, CD3E, CD3G, CD5, EDF1, FTL, PTEN, PTPN2, PTPN6, PTPRC, PTPRCAP, RPS23, RTRAF, SERF2, SLC38A1, SMAD2, SOCS1, SRP14, SRSF9, SUB1, TET2, TIGIT, TRAC, or TRIM28.

In some embodiments, the one or more gRNAs comprises any one of SEQ ID NOS: 1-120.

In some embodiments, the cell is CD45RA⁺ and CCR7⁺ after insertion of the at least one sequence into the safe harbor locus.

In some embodiments, the DNA template is a double-stranded DNA template or a single-stranded DNA template.

In some embodiments, the DNA template is a linear DNA template or a circular DNA template, optionally wherein the circular DNA template is a plasmid.

In some embodiments, the DNA template comprises a heterologous sequence.

In some embodiments, the DNA template comprises a gene.

In some embodiments, the DNA template comprises a priming receptor comprising a transcription factor.

In some embodiments, the DNA template comprises a chimeric antigen receptor (CAR).

In some embodiments, the DNA template comprises a chimeric antigen receptor

(CAR) and a priming receptor comprising a transcription factor.

In some embodiments, the DNA template comprises an inducible promoter operably linked to the chimeric antigen receptor.

In some embodiments, the DNA template further comprises a constitutive promoter operably linked to the priming receptor.

In some embodiments, the DNA template further comprises an inducible promoter operably linked to the chimeric antigen receptor and a constitutive promoter operably linked to the priming receptor.

In some embodiments, the DNA template comprises, in a 5′ to 3′ direction:
i. the inducible promoter;
ii. the chimeric antigen receptor;
iii, the constitutive promoter; and
iv. the priming receptor.

In some embodiments, the DNA template comprises, in a 5′ to 3′ direction:
i. the constitutive promoter;
ii. the priming receptor;
iii, the inducible promoter; and
iv. the chimeric antigen receptor.

In some embodiments, the priming receptor comprises, in an N terminus to C terminus direction:
i. an extracellular antigen-binding domain having a binding affinity for an antigen;
ii. a transmembrane domain comprising one or more ligand-inducible proteolytic cleavage sites; and
iii. an intracellular domain comprising a human or humanized transcriptional effector, wherein binding of an antigen to the extracellular antigen-binding domain results in cleavage at the ligand-inducible proteolytic cleavage site thereby releasing the intracellular domain.

In some embodiments, the priming receptor further comprises a juxtamembrane domain (JMD) or stop transfer sequence (STS) positioned between the transmembrane domain and the intracellular domain.

In some embodiments, the transcription factor binds to the inducible promoter and induces expression of the CAR.

In some embodiments, the CAR comprises, from N-terminus to C-terminus,
i. an extracellular antigen-binding domain having a binding affinity for an antigen;
ii. a transmembrane domain;
iii. an intracellular co-stimulatory domain; and
iv. an intracellular activation domain.

In some embodiments, the priming receptor and the CAR bind different antigens.

In some embodiments, the priming receptor and the CAR bind the same antigen.

In one aspect, provided herein are methods of editing an immune cell, comprising:
i. providing an immune cell comprising an exogenous homologous recombination or DNA repair modulation protein;
ii. providing a solution comprising a ribonucleoprotein complex (RNP), a DNA template, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the cell; and
iii. editing the cell via insertion of the DNA template into the insertion site in the genome of the cell.

In one aspect, provided herein are methods of editing an immune cell, comprising:
i. providing an immune cell comprising an exogenous homologous recombination or DNA repair modulation protein;
ii. providing a solution comprising a ribonucleoprotein complex (RNP), a DNA template, and at least one of an RNase inhibitor, N acetyl-L-cysteine (NAC), an osmolarity-adjusting agent and/or a histone deacetylase (HDAC) inhibitor, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the cell; and
iii. editing the immune cell via insertion of the DNA template into the insertion site in the genome of the cell.

In some embodiments, the exogenous homologous recombination or DNA repair modulation protein is SWSAP1, a dominant negative KU80, or an AcrIIA8-CDT1 fusion protein.

In some embodiments, the SWSAP1 protein comprises the sequence as set forth in SEQ ID NO: 125; the dominant negative KU80 protein comprises the sequence as set forth in SEQ ID NO: 126, or the AcrIIA8-CDT1 fusion protein comprises the sequence as set forth in SEQ ID NO: 127.

In one aspect, provided herein are methods of editing an immune cell, comprising:
i. providing a solution comprising an RNase inhibitor, a ribonucleoprotein complex (RNP), and a DNA template, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the cell;
ii. non-virally introducing the RNP and DNA template into the immune cell, wherein the guide RNA specifically hybridizes to a target region of the genome of the cell, and wherein the nuclease domain cleaves the target region to create the insertion site in the genome of the cell; and
iii. editing the immune cell via insertion of the DNA template into the insertion site in the genome of the cell.

In one aspect, provided herein are methods of editing an immune cell, comprising:
i. providing a solution comprising N acetyl-L-cysteine (NAC), a ribonucleoprotein complex (RNP), and a DNA template wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the immune cell;
ii. non-virally introducing the RNP and DNA template into the immune cell, wherein the guide RNA specifically hybridizes to a target region of the genome of the cell, and wherein the nuclease domain cleaves the target region to create the insertion site in the genome of the cell; and
iii. editing the immune cell via insertion of the DNA template into the insertion site in the genome of the cell.

In one aspect, provided herein are methods of editing an immune cell, comprising:
i. providing a solution comprising an osmolarity-adjusting agent, a ribonucleoprotein complex (RNP), and a DNA template, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the immune cell;
ii. non-virally introducing the RNP and DNA template into the immune cell, wherein the guide RNA specifically hybridizes to a target region of the genome of the cell, and wherein the nuclease domain cleaves the target region to create the insertion site in the genome of the cell; and
iii. editing the cell via insertion of the DNA template into the insertion site in the genome of the cell.

In one aspect, provided herein are methods of editing an immune cell, comprising:
i. providing a solution comprising a histone deacetylase (HDAC) inhibitor comprising at least one of sodium phenylbutyrate, quisinostat, or panobinostat, a ribonucleoprotein complex (RNP), and a DNA template, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the immune cell;
ii. non-virally introducing the RNP and DNA template into the cell, wherein the guide RNA specifically hybridizes to a target region of the genome of the cell, and wherein the nuclease domain cleaves the target region to create the insertion site in the genome of the cell; and
iii. editing the cell via insertion of the DNA template into the insertion site in the genome of the cell.

In one aspect, provided herein are methods method of editing an immune cell, comprising:
i. providing a solution comprising a ribonucleoprotein complex (RNP), a DNA template, and at least one of an RNase inhibitor, N acetyl-L-cysteine (NAC), an osmolarity-adjusting agent, and/or a histone deacetylase (HDAC) inhibitor, wherein the RNP comprises a nuclease domain and a guide RNA, wherein the DNA template comprises a chimeric antigen receptor (CAR) and a priming receptor comprising a transcription factor, wherein the 5′ and 3′ ends of the DNA template comprise nucleotide sequences that are homologous to genomic sequences flanking an insertion site in the genome of the cell;
ii. non-virally introducing the RNP and DNA template into the immune cell, wherein the guide RNA specifically hybridizes to a target region of the genome of the cell, and wherein the nuclease domain cleaves the target region to create the insertion site in the genome of the cell; and
iii. editing the cell via insertion of the DNA template into the insertion site in the genome of the cell.

In one aspect, provided herein are methods of treating a subject having or at risk of having a disease, comprising:
i. conducting a method disclosed herein; and
ii. administering to the subject an effective amount of a composition comprising the cell or a population thereof.

In some embodiments, the composition is administered to the subject by infusion.

In some embodiments, the disease is cancer.

In some embodiments, immune cells produced by a method disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows the percent knock in of an exemplary transgene after electroporation in the presence (+) or absence (−) of an RNase inhibitor. FIG. 1B shows the expansion fold increase of T cells after electroporation in the presence (+) or absence (−) of an RNase inhibitor. FIG. 1C shows the normalized edited T cell yield after electroporation in the presence (+) or absence (−) of an RNase inhibitor.

FIG. 2A shows the % knock-in (KI) of an exemplary transgene after electroporation of control cells (left bars) and cells treated with an RNase inhibitor (right cells) for cells from two different donors using the Xenon MultiShot platform at clinical-scale. FIG. 2B shows the total number of edited cells after electroporation of control cells (left bars) and cells treated with an RNase inhibitor (right cells) for cells from two different donors using the Xenon MultiShot platform at clinical-scale.

FIG. 3 shows the fold change in edited T cell yield (top panels) and the fold change in KI (bottom panels) over control cells as a function of cell and payload exposure time, electroporated with (top line) and without (bottom line) the addition of RNase inhibitor using the Xenon platform. Control cells were electroporated without RNase inhibitor with a zero min cell and payload exposure time. Data shown is from five different donors. Errors bars=standard error of the mean

FIG. 4A shows the fold change in gene knock in (KI) in T cells electroporated with and without the addition of RNase inhibitor in five different donor cells using the Xenon platform. FIG. 4B shows the fold change in edited cell yield over control cells with the addition of RNase inhibitor in five different donor cells using the Xenon platform. Control cells were electroporated without RNase inhibitor with a zero min cell and payload exposure time (dotted line). Data shown is averaged from five different donors. Errors bars=standard error of the mean.

FIG. 5A shows the proportion of stem cell memory T cells (Tscm) and central memory T cells (Tcm), defined by CCR7 and CD45RA expression, of T cells electroporated in the presence or absence of an RNase inhibitor in cells from 13 different donors using the Xenon platform. FIG. 5B shows the ratio of CD4 to CD8 T cells after electroporation in the presence or absence of an RNase inhibitor in cells from the same 13 different donors. p-values are from a two-sided matched pairs t test.

FIG. 6A shows the fold change in edited cell yield in cells from five donors after electroporation using the Lonza platform in the presence of 0 mM or 5 mM NAC. FIG. 6B shows the precent KI of the transgene yield in cells from five donors after electroporation in the presence of 0 mM (left bars) or 5 mM NAC (right bars).

FIG. 7A shows the fold change in edited cell yield in cells from three donors after electroporation using the Xenon platform in the presence of 0 mM or 5 mM NAC. FIG. 7B shows the precent KI of the transgene yield in cells from three donors after electroporation using the Xenon platform in the presence of 0 mM (left bars) or 5 mM NAC (right bars). FIG. 7C shows the normalized edited cell yield in cells from three donors after electroporation using the Xenon platform in the presence of 0 mM (left bars) or 5 mM NAC (right bars).

FIG. 8A shows the percent KI in cells after electroporation with 2.5 mM NAC or 5 mM NAC, across five different donors. FIG. 8B shows the total edited cells after electroporation with 2.5 mM NAC or 5 mM NAC, across five different donors.

FIG. 9A shows the fold changes in edited cell yields after electroporation using the Xenon platform with 200 mM sorbitol, across five different donors. FIG. 9B compares editing efficiencies with and without 200 mM sorbitol during electroporation using the Xenon platform, across the same donors.

FIG. 10A shows the correlation in KI efficiency between two donor cell lines electroporated with plasmid transiently expressing the indicated proteins. FIG. 10B shows the correlation in KI efficiency between two donor cell lines electroporated with plasmid transiently expressing the indicated proteins. Luciferase was used as a control. NLS tags are noted where applicable.

FIG. 11A shows bar graphs of the % KI efficiency between cells from two donors using a 9 kb plasmid transiently expressing a GFP reporter. FIG. 11B shows bar graphs of the % KI efficiency between cells from two donors using a 9 kb plasmid transiently expressing an exemplary myc-tagged CAR. In both cases a control plasmid encoding a luciferase reporter gene (luc) was also used.

FIG. 12 show the fold change in knock in (KI) percent of an exemplary transgene in T cells after incubation of the T cells with 0.5 mM, 1 mM, 2 mM and 4 mM sodium phenylbutyrate with 1% DMSO in culture media after electroporation.

FIG. 13 show the in knock in (KI) percent of an exemplary transgene in T cells after pre-incubation of the T cells with 0.1 mM sodium phenylbutyrate for 2 days prior to electroporation and recovery in standard culture medium versus recovery with 1 mM sodium phenylbutyrate with 1% DMSO in the culture medium.

FIG. 14 shows the knock in (KI) percent of an exemplary transgene in T cells after incubation of the T cells with 0.016 μM (16 nM) Quisinostat or 0.0375 μM (37.5 nM) Panobinostat after electroporation.

FIG. 15 shows the knock in (KI) percent of an exemplary transgene in T cells after incubation of the T cells with the indicated compounds.

FIG. 16A shows the % knock in (KI) and total edited T cells from seven different donor expressing the exemplary transgene(s) after 4 or 5 electroporation pulses in media supplemented with xeno-free Supplement B. The 4 pulse sample is shown in the left bar, the 5 pulse sample is shown in the right bar. FIG. 16B shows the % knock in (KI) and total edited T cells from five different donor expressing the exemplary transgene(s) after 4 or 5 electroporation pulses in media supplemented with xeno-free Supplement A.

FIG. 17A shows the number of tumor target cells after incubation with edited T cells at a 1:50 E:T ratio. FIG. 17B shows the number of tumor target cells after incubation with edited T cells at a 1:100 E:T ratio.

FIG. 18 shows that % KI and cell expansion (TECs) on Days 7 and 9 post electroporation was increased in the 5-pulse protocol as compared to the 4-pulse protocol in T cells from five donors.

FIG. 19A shows % KI and total edited T cells after 5 electroporation pulses (middle bars), 4 electroporation pulses (left bar) and 3 electroporation pulses (right bar) with a nanoplasmid (reduced backbone length) encoding an exemplary transgene. FIG. 19B shows % KI and total edited T cells after 5 electroporation pulses (middle bars), 4 electroporation pulses (left bar) and 3 electroporation pulses (right bar) with a standard plasmid (full backbone length) encoding an exemplary transgene.

FIG. 20A shows the % KI and total number of edited T cells after electroporation with 4 pulses or 5 pulses. Cells were incubated in media containing Xeno-Free Supplement A. FIG. 20B shows the % KI and total number of edited T cells after electroporation with 4 pulses or 5 pulses. Cells were incubated in media containing Xeno-Free Supplement B.

### DETAILED DESCRIPTION

### Definitions

Terms used in the claims and specification are defined as set forth below unless otherwise specified.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS). As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table 1.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-directed nuclease, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24; 110 (39): 15644-9; Sampson et al., Nature. 2013 May 9; 497 (7448): 254-7; and Jinek, et al., Science. 2012 Aug. 17; 337 (6096): 816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-guided nucleases include the foregoing Cas9 proteins and homologs thereof, and include but are not limited to, CPF1 (See, e.g., Zetsche et al., Cell, Volume 163, Issue 3, p 759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+double positive T cells, CD4+ helper T cells (e.g. Th1 and Th2 cells), CD8+ T cells (e.g. cytotoxic T cells), peripheral T cells, including but not limited to peripheral blood mononuclear cells (PBMC) and peripheral blood leukocytes (PBL), tumor infiltrating lymphocytes (TIL), memory T cells, stem memory T cells (Tscm), effector T cells, naive T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin. In some cases, human hematopoietic stem cells are identified as CD34⁺, CD59⁺, Thy 1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy 1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the phrase “immune cell” is inclusive of all cell types that give rise to immune cells, including hematopoietic cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell, a hematopoietic stem and progenitor cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3 agonists, CD28 agonists, IL-2, IL-7, IL-15, IFN-γ, or a combination thereof.

As used herein, the term “ex vivo” generally includes experiments or measurements made in or on living tissue, preferably in an artificial environment outside the organism, preferably with minimal differences from natural conditions.

As used herein, the term “construct” refers to a complex of molecules, including macromolecules or polynucleotides. For example, a construct can be a DNA polynucleotide molecule created through artificial means. In some embodiments, a DNA construct can be propagated via plasmid replication in bacteria.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. Portions of the, or the whole, polynucleotide donor construct are transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide or protein. It is optionally translated into a recombinant protein. A “recombinant protein” is a protein encoded by a gene—recombinant or synthetic DNA—that has been cloned in a system that supports expression of the gene and translation of messenger RNA (see expression system). The protein can be a therapeutic agent, e.g. a protein that treats a disease or disorder disclosed herein, such as a CAR, a priming receptor, or a TCR. As used, transgene can refer to a polynucleotide that encodes a polypeptide or protein. A transgene can also refer to a non-protein encoding polynucleotide sequence, such as, but not limited to shRNAs, siRNAs, miRNAs, and miRs.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein, polypeptide, or polynucleotide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. In some embodiments, the original sequence is replaced with the sequence of the template. In some embodiments, the sequence of the template is inserted into the genome without replacing an endogenous sequence. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

It must be noted that, as used in the specification and the appended claims, the singular forms “a,” “an” and “the” include plural referents unless the context clearly dictates otherwise.

### Compositions for Editing Cells

In some aspects, provided herein are compositions comprising a solution comprising a cell comprising at least one heterologous DNA template inserted into a target region of the genome, and at least one of an RNase inhibitor, N acetyl-L-cysteine (NAC), a histone deacetylase (HDAC) inhibitor, and/or an osmolarity-adjusting agent. In some embodiments, the solution comprises a cell comprising at least one heterologous DNA template inserted into a target region of the genome, and N acetyl-L-cysteine (NAC). In some embodiments, the solution comprises a solution comprising a cell comprising at least one heterologous DNA template inserted into a target region of the genome, and a histone deacetylase (HDAC) inhibitor. In some embodiments, the solution is an aqueous solution. In some embodiments, the at least one of an RNase inhibitor, N acetyl-L-cysteine (NAC), a histone deacetylase (HDAC) inhibitor is present in the solution at a final concentration as described herein.

In some embodiments, the solution comprises a buffer. In some embodiments, the solution comprises cell media. In some embodiments, the cell media comprises a media supplement. In some embodiments, the cell media comprises a xeno-media supplement. Exemplary cell media supplements include, but are not limited to, human serum, fetal bovine serum (FBS), Cell-Vive™ T-NK Xeno-Free Serum (BioLegend), Cell-Vive™ T cell CD Serum Substitute (BioLegend), PLT Gold (Sarotrius), Serum Substitute Supplement (Irvine Scientific), Physiologix™ XF Serum Replacement (Nucleus Biologics), CTS™ Immune Cell Serum Replacement (Thermo Fisher Scientific), Knock Out Serum Replacement (Thermo Fisher Scientific). In some embodiments, the media supplement comprises Cell-Vive™ T-NK Xeno-Free Serum, Cell-Vive™ T cell CD Serum Substitute, PLT Gold, Serum Substitute Supplement, Physiologix™ XF Serum Replacement, CTS™ Immune Cell Serum Replacement, Knock Out Serum Replacement, fetal bovine serum, or human serum.

### RNase Inhibitor

In some aspects, provided herein are compositions comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell. Also provided are methods of editing a cell, comprising providing a solution comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell

In some embodiments, the RNase inhibitor is present in the solution at a final concentration of between about 0.5 to 2 U/μl. The final concentration of the RNase inhibitor in the solution can be about 0.5 U/μl, 0.75 U/μl, 1 U/μl, 1.25 U/μl, 1.5 U/μl, 1.75 U/μl, or 2 U/μl. The final concentration of the RNase inhibitor in the solution can be between about 0.5-2 U/μl, 0.5-1 U/μl, 0.5-0.75 U/μl, 0.75-1 U/μl, 1-1.25 U/μl, 1.25-1.5 U/μl, 1.5-1.75 U/μl, or 1.75-2 U/μl. In some embodiments, the RNase inhibitor is present in the solution at a final concentration of about 1 U/μL. In some embodiments, the RNase inhibitor is a murine RNase inhibitor. In some embodiments, the RNase inhibitor is an RNase A, B, C, T1, or T2 inhibitor. In some embodiments, the RNase inhibitor is a murine, rat, or human RNase inhibitor.

RNase inhibitors are generally commercially available from a variety of vendors, such as Thermo Fisher (catalogue no. AM2694), Invitrogen (catalogue no. EO0381), Promega (catalogue no. N2111), New England Biolabs (catalogue no. M0314S or M0314L), Takara, and Sigma-Aldrich. In some embodiments, the RNase inhibitor specifically inhibits RNases A, B, C, 1, or T1.

In some embodiments, the RNase inhibitor is added to a solution. In some embodiments, the RNase inhibitor is added to an aqueous solution. In some embodiments, the RNase inhibitor is added to a buffer. In some embodiments, the composition comprising the RNase inhibitor can also further comprise an osmolarity-adjusting agent and/or N acetyl-L-cysteine (NAC) as described herein. In some embodiments, the composition comprising the RNase inhibitor further comprises an osmolarity-adjusting agent. In some embodiments, the composition comprising the RNase inhibitor further comprises N acetyl-L-cysteine (NAC). In some embodiments, the composition comprising the RNase inhibitor further comprises an osmolarity-adjusting agent and N acetyl-L-cysteine (NAC).

### Osmolarity-Adjusting Agent

In some aspects, provided herein are compositions comprising a solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell. Also provided are methods of editing a cell, comprising providing solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some embodiments, the osmolarity-adjusting agent is sorbitol, glycerol, or glycine. In some embodiments, the osmolarity-adjusting agent is two or more of sorbitol, glycerol, or glycine. In some embodiments, the osmolarity-adjusting agent is sorbitol. In some embodiments, the osmolarity-adjusting agent is glycerol. In some embodiments, the osmolarity-adjusting agent is glycine.

In some embodiments, the osmolarity-adjusting agent is present in the solution at a final concentration of between about 100-400 mM. In some embodiments, the osmolarity-adjusting agent is present in the solution at a final concentration of between about 100-150 mM, 150-200 mM, 200-250 mM, 250-300 mM, 300-350 mM, or 350-400 mM. In some embodiments, the osmolarity-adjusting agent is present in the solution at a final concentration of about 100 mM, 125 mM, 150 mM, 175 mM, 190 mM, 200 mM, 225 mM, 250 mM, 275 mM, 300 mM, 325 mM, 350 mM, 375 mM, or 400 mM.

In some embodiments, the osmolarity-adjusting agent is present in the solution at a final concentration of about 0.5%-1.5%, 0.5%-1%, 1%-1.25%, or 1.25%-1.5%. In some embodiments, the osmolarity-adjusting agent is present in the solution at a final concentration of about 1.25%. In some embodiments, the osmolarity-adjusting agent is present in the solution at a final concentration of less than 2%, 1.9%, 1.8%, 1.7%, 1.6%, or 1.5%.

In some embodiments, the sorbitol is present in the solution at a final concentration of between about 100-400 mM. In some embodiments, the sorbitol is present in the solution at a final concentration of between about 100-400 mM. In some embodiments, the sorbitol is present in the solution at a final concentration of between about 100-150 mM, 150-200 mM, 200-250 mM, 250-300 mM, 300-350 mM, or 350-400 mM. In some embodiments, the sorbitol is present in the solution at a final concentration of about 100 mM, 125 mM, 150 mM, 175 mM, 190 mM, 200 mM, 225 mM, 250 mM, 275 mM, 300 mM, 325 mM, 350 mM, 375 mM, or 400 mM. In some embodiments, the sorbitol is present in the solution at a final concentration of about 190 mM or 200 mM. In some embodiments, the sorbitol is present in the solution at a final concentration of about 200 mM. In some embodiments, the sorbitol is present in the solution at a final concentration of about 170 mM.

In some embodiments, the sorbitol is present in the solution at a final concentration of about 0.5%-1.5%, 0.5%-1%, 1%-1.25%, or 1.25%-1.5%. In some embodiments, the sorbitol is present in the solution at a final concentration of about 1.25%. In some embodiments, the sorbitol is present in the solution at a final concentration of less than 2%, 1.9%, 1.8%, 1.7%, 1.6%, or 1.5%.

In some embodiments, the glycerol is present in the solution at a final concentration of between about 100-400 mM. In some embodiments, the glycerol is present in the solution at a final concentration of between about 100-400 mM. In some embodiments, the glycerol is present in the solution at a final concentration of between about 100-150 mM, 150-200 mM, 200-250 mM, 250-300 mM, 300-350 mM, or 350-400 mM. In some embodiments, the glycerol is present in the solution at a final concentration of about 100 mM, 125 mM, 150 mM, 175 mM, 190 mM, 200 mM, 225 mM, 250 mM, 275 mM, 300 mM, 325 mM, 350 mM, 375 mM, or 400 mM. In some embodiments, the glycerol is present in the solution at a final concentration of about 190 mM or 200 mM. In some embodiments, the glycerol is present in the solution at a final concentration of about 200 mM. In some embodiments, the glycerol is present in the solution at a final concentration of about 170 mM.

In some embodiments, the glycerol is present in the solution at a final concentration of about 0.5%-1.5%, 0.5%-1%, 1%-1.25%, or 1.25%-1.5%. In some embodiments, the glycerol is present in the solution at a final concentration of about 1.25%. In some embodiments, the glycerol is present in the solution at a final concentration of less than 2%, 1.9%, 1.8%, 1.7%, 1.6%, or 1.5%.

In some embodiments, the glycine is present in the solution at a final concentration of between about 100-400 mM. In some embodiments, the glycine is present in the solution at a final concentration of between about 100-400 mM. In some embodiments, the glycine is present in the solution at a final concentration of between about 100-150 mM, 150-200 mM, 200-250 mM, 250-300 mM, 300-350 mM, or 350-400 mM. In some embodiments, the glycine is present in the solution at a final concentration of about 100 mM, 125 mM, 150 mM, 175 mM, 190 mM, 200 mM, 225 mM, 250 mM, 275 mM, 300 mM, 325 mM, 350 mM, 375 mM, or 400 mM. In some embodiments, the glycine is present in the solution at a final concentration of about 190 mM or 200 mM. In some embodiments, the glycine is present in the solution at a final concentration of about 200 mM. In some embodiments, the glycine is present in the solution at a final concentration of about 170 mM.

In some embodiments, the glycine is present in the solution at a final concentration of about 0.5%-1.5%, 0.5%-1%, 1%-1.25%, or 1.25%-1.5%. In some embodiments, the glycine is present in the solution at a final concentration of about 1.25%. In some embodiments, the glycine is present in the solution at a final concentration of less than 2%, 1.9%, 1.8%, 1.7%, 1.6%, or 1.5%.

In some embodiments, the osmolarity-adjusting agent is added to a solution. In some embodiments, the osmolarity-adjusting agent is added to an aqueous solution. In some embodiments, the osmolarity-adjusting agent is added to a buffer. In some embodiments, the composition comprising the osmolarity-adjusting agent can also further comprise the N acetyl-L-cysteine (NAC) and/or an RNase inhibitor as described herein. In some embodiments, the composition comprising the osmolarity-adjusting agent (e.g., sorbitol, glycerol, or glycine) further comprises N acetyl-L-cysteine (NAC). In some embodiments, the composition comprising the osmolarity-adjusting agent (e.g., sorbitol, glycerol, or glycine) further comprises an RNase inhibitor. In some embodiments, the composition comprising the osmolarity-adjusting agent (e.g., sorbitol, glycerol, or glycine) further comprises N acetyl-L-cysteine (NAC) and an RNase inhibitor.

### Reactive Oxygen Species (ROS) Inhibitors

In some aspects, provided herein are compositions comprising a solution comprising a cell comprising at least one heterologous DNA template inserted into a target region of the genome, and a reactive oxygen species (ROS) inhibitor (e.g., N acetyl-L-cysteine (NAC)). In some embodiments, the solution comprises a cell comprising at least one heterologous DNA template inserted into a target region of the genome, and N acetyl-L-cysteine (NAC). In some embodiments, the solution is an aqueous solution. In some embodiments, the reactive oxygen species (ROS) inhibitor (e.g., N acetyl-L-cysteine (NAC)), is present in the solution at a final concentration as described herein.

In some aspects, provided herein are compositions comprising a solution comprising a reactive oxygen species (ROS) inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell. Also provided are methods of editing a cell, comprising providing a solution comprising reactive oxygen species (ROS) inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell. In some embodiments, a reactive oxygen species (ROS) inhibitor is a reactive oxygen species (ROS) scavenger.

Exemplary ROS inhibitors include, but are not limited to, N acetyl-L-cysteine (NAC), N-Acetyl-D-cysteine, quercetin, Deferoxamine mesylate, Phycocyanobilin, Mito-TEMPO, GSK2795039, Diphenyleneiodonium chloride, Tempol, Succinyl phosphonate trisodium salt, Nobiletin, Albendazole, Imeglimin, N-tert-Butyl-α-phenylnitrone, Tofogliflozin (hydrate), glutathione, butylated hydroxyanisole (BHA), butylated hydroxytoluene (BHT), ascorbic acid, tocopherol, α-Tocopherol phosphate disodium, Tofogliflozin (hydrate), Lacidipine, Astaxanthin, Spiraeoside, Moracin O, Uric acid sodium, Clovamide, Norbergenin, L-Theanine, Sodium 2-oxopropanoate, 3,4-Dimethoxycinnamic acid, Aureusidin, Pelargonidin chloride, Randialic acid B, Cyclo(L-Phe-L-Pro), Decylubiquinone, Bixin, Sodium thiocyanate, 5-Hydroxyoxindole, and Simvastatin hydroxy acid sodium. Additional ROS inhibitors are commercially available from at least MedChem express (medchemexpress.com/Targets/reactive-oxygen-species/effect/inhibitor.html) and Selleckchem (selleckchem.com/ROS.html).

In some aspects, provided herein are compositions comprising a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell. Also provided are methods of editing a cell, comprising providing a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some embodiments, the N acetyl-L-cysteine (NAC) is present in the solution at a final concentration of between about 1-10 mM. In some embodiments, the N acetyl-L-cysteine (NAC) is present in the solution at a final concentration of between about 1-10 mM, 1-2 mM, 2-3 mM, 3-4 mM, 4-5 mM, 5-6 mM, 6-7 mM, 7-8 mM, 8-9 mM, or 9-10 mM. In some embodiments, the N acetyl-L-cysteine (NAC) is present in the solution at a final concentration of about 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, or 10 mM. In some embodiments, the N acetyl-L-cysteine (NAC) is present in the solution at a final concentration of about 5 mM. In some embodiments, the N acetyl-L-cysteine (NAC) is present in the solution at a final concentration of about 2.5 mM.

N acetyl-L-cysteine (NAC) (CAS no. 616-91-1), also known as 2-acetamido-3-sulfanylpropanoic acid (IUPAC name) is an antioxidant and mucolytic agent. It is a precursor of the antioxidant glutathione and can increase cellular pools of free radical scavengers. NAC is commercially available from various vendors, including, but not limited to, Sigma Aldrich (catalogue no. A7250), Thermo Fisher (catalogue no. A15409.36), Santa Cruz Biotechnology (catalogue no. sc-202232), or Padagis (catalog no. NDC 0574-0805-30).

In some embodiments, the N acetyl-L-cysteine (NAC) is added to a solution. In some embodiments, the N acetyl-L-cysteine (NAC) is added to an aqueous solution. In some embodiments, the N acetyl-L-cysteine (NAC) is added to a buffer. In some embodiments, the composition comprising the N acetyl-L-cysteine (NAC) can also further comprise an osmolarity-adjusting agent and/or an RNase inhibitor as described herein. In some embodiments, the composition comprising the N acetyl-L-cysteine (NAC) further comprises an osmolarity-adjusting agent. In some embodiments, the composition comprising the N acetyl-L-cysteine (NAC) further comprises an RNase inhibitor. In some embodiments, the composition comprising the N acetyl-L-cysteine (NAC) further comprises an osmolarity-adjusting agent and an RNase inhibitor. In some embodiments, the solution comprises at least one of a final concentration of about 0.5-2 U/μL RNase inhibitor, a final concentration of about 1-10 mM NAC, and/or a final concentration of about 100-400 mM sorbitol. In some embodiments, the solution comprises at least one of a final concentration of about 1 U/μL RNase inhibitor, a final concentration of about 5 mM NAC, and/or a final concentration of about 200 mM sorbitol. In some embodiments, the solution comprises at least one of a final concentration of about 1 U/μL RNase inhibitor, a final concentration of about 2.5 mM NAC, and/or a final concentration of about 200 mM sorbitol. In some embodiments, the solution comprises at least one of a final concentration of about 1 U/μL RNase inhibitor, a final concentration of about 5 mM NAC, and/or a final concentration of about 170 mM glycerol. In some embodiments, the solution comprises at least one of a final concentration of about 1 U/μL RNase inhibitor, a final concentration of about 2.5 mM NAC, and/or a final concentration of about 170 mM glycerol.

### Histone Deacetylase (HDAC) Inhibitors

In some aspects, provided herein are compositions comprising a solution comprising a histone deacetylase (HDAC) inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In some aspects, provided herein are compositions comprising a solution comprising a cell comprising at least one heterologous DNA template inserted into a target region of the genome, and a histone deacetylase (HDAC) inhibitor described herein. In some embodiments, the solution is an aqueous solution. In some embodiments, the histone deacetylase (HDAC) inhibitor is present in the solution at a final concentration as described herein.

In some aspects, provided herein are compositions comprising a solution comprising a histone deacetylase (HDAC) inhibitor comprising at least one of sodium phenylbutyrate, quisinostat, or panobinostat, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

The histone deacetylase (HDAC) inhibitor can be an HDAC1, HDAC2, HDAC3, HDAC4, HDAC5, HDAC6, HDAC7, HDAC8, HDAC9, and/or HDAC10 inhibitor. In certain embodiments, the HDAC inhibitor is a broad spectrum HDAC inhibitor. Exemplary HDAC inhibitors include, but are not limited to, sodium phenylbutyrate, quisinostat, Panobinostat, phenylbutyric acid, curcumin, mocetinostat, romidespin, SIS17, splitomicin, trichostatin-A, tucidinostat, etinostat, sodium butyrate, valproic acid, CXD101, KT-531, ITF3756, tubastatin A, vorinostat, BML-210, belinostat, Abexinostat, dacinostat, CUDC-101, droxinostat, MC1568, pracinostat, divalproex sodium, PCI-3405, SR-4370, givinostat, tubacin, AR-42, (−)-parthenolide, resminostat, fimepinostat, M344, tacedinaline, sinapinic acid, biphenyl-4-sufonyl chloride, sulforaphane, UF010, suberohdroxamic acid, NKL 22, TC-H 106, RGFP966, HPOB, RG2833, TMP269, nexturastat A, domatinostat, LMK-235, santacruzamate A, CAY10603, tasquinimod, BG45, BRD73954, ricolinostat, scriptaid, citarinostat, WT161, TMP195, ACY-738, SKLB-23bb, tinostamustine, TH34, BRD3308, raddeanin A, isoguanosine, KA2507, ITSA-1, or an RNA interference (RNAi) molecule. In some embodiments, the HDAC inhibitor is sodium phenylbutyrate, quisinostat or panobinostat.

In some embodiments, the sodium phenylbutyrate is present in the solution at a final concentration of about 1 mM or between about 15.6 μM-4 mM, optionally wherein the solution comprises 1% DMSO. In some embodiments, the sodium phenylbutyrate is present in the solution at a final concentration of about 1 mM or between about 15.6 μM-4 mM, and 1% DMSO. In some embodiments, the sodium phenylbutyrate is present in the solution at a final concentration of about 15 μM, 50 μM, 100 μM, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, 1 mM, 1.5 mM, 2 mM, 2.5 mM, 3 mM, 3.5 mM, or 4 mM, or between about 15.6 μM-4 mM, 15 μM-50 μM, 50 μM-100 μM, 100 μM-150 μM, 150 μM-200 μM, 200 μM-250 μM, 250 μM-300 μM, 300 μM-350 μM, 350 μM-400 μM, 400 μM-450 μM, 450 μM-500 μM, 500 μM-550 μM, 550 μM-600 μM, 600 μM-650 μM, 650 μM-700 μM, 750 μM-800 μM, 850 μM-900 μM, 900 μM-950 μM, 950 μM-1000 μM, 1-1.25 mM, 1.25-1.5 mM, 1.5-1.75 mM, 1.75-2 mM, 2-2.25 mM, 2.25-2.5 mM, 2.5-2.75 mM, 2.75-3 mM, 3-3.25 mM, 3.25-3.5 mM, or 3.5-4 mM, or any concentration in between. In some embodiments, the sodium phenylbutyrate is present in the solution at a final concentration of about 0.1 mM.

In some embodiments, the quisinostat is present in the solution at a final concentration of about 16 nM or between about 8 nM-200 nM. In some embodiments, the quisinostat is present in the solution at a final concentration of about 8 nM, 9 nM, 10 nM, 11 nM, 12 nM, 13 nM, 14 nM, 15 nM, 16 nM, 17 nM, 18 nM, 19 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 120 nM, 125 nM, 130 nM, 135 nM, 140 nM, 145 nM, 150 nM, 155 nM, 160 nM, 165 nM, 170 nM, 175 nM, 180 nM, 185 nM, 190 nM, 195 nM, or 200 nM, or between about 8-10 nM, 10-12 nM, 10-14 nM, 14-16 nM, 16-18 nM, 18-20 nM, 20-25 nM, 25-30 nM, 30-35 nM, 35-40 nM, 40-45 nM, 45-50 nM, 50-55 nM, 55-60 nM, 60-65 nM, 65-70 nM, 70-75 nM, 75-80 nM, 80-85 nM, 85-90 nM, 90-95 nM, 95-100 nM, 100-105 nM, 105-110 nM, 110-120 nM, 120-125 nM, 125-130 nM, 130-135 nM, 135-140 nM, 140-145 nM, 145-150 nM, 150-155 nM, 155-160 nM, 160-165 nM, 165-170 nM, 170-175 nM, 175-180 nM, 180-185 nM, 185-190 nM, 190-195 nM, or 195-200 nM, or any concentration in between.

In some embodiments, the panobinostat is present in the solution at a final concentration of about 37.5 nM or between about 3 nM-1 μM. In some embodiments, the panobinostat is present in the solution at a final concentration of about 3 nM, 5 nM, 10 nM, 15 nM, 20 nM, 25 nM, 30 nM, 35 nM, 37.5 nM, 40 nM, 45 nM, 50 nM, 55 nM, 60 nM, 65 nM, 70 nM, 75 nM, 80 nM, 85 nM, 90 nM, 95 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1000 nM, or between about 3-5 nM, 5-10 nM, 10-20 nM, 20-30 nM, 30-35 nM, 35-37.5 nM, 35-40 nM, 40-45 nM, 45-50 nM, 55-60 nM, 60-65 nM, 65-70 nM, 70-75 nM, 75-80 nM, 85-90 nM, 90-95 nM, 95-100 nM, 100-125 nM, 125-150 nM, 150-175 nM, 175-200 nM, 200-225 nM, 225-250 nM, 250-275 nM, 275-200 nM, 300-325 nM, 325-350 nM, 350-375 nM, 375-300 nM, 400-425 nM, 425-450 nM, 450-475 nM, 475-400 nM, 500-525 nM, 525-550 nM, 550-575 nM, 575-500 nM, 600-625 nM, 625-650 nM, 650-675 nM, 675-600 nM, 700-725 nM, 725-750 nM, 750-775 nM, 775-700 nM, 800-825 nM, 825-850 nM, 850-875 nM, 875-800 nM, 900-925 nM, 925-950 nM, 950-975 nM, or 975-1000 nM, or any concentration in between.

In some embodiments, the solution comprises at least one of a final concentration of about 0.5 to 2 U/μl RNase inhibitor, a final concentration of about 1-10 mM NAC, a final concentration of about 100-400 mM sorbitol, and/or a final concentration of about 15.6 μM-4 mM sodium phenylbutyrate, 8 nM-200 nM quisinostat, or 3 nM-1 μM Panobinostat.

### Combinations

A composition comprising a solution comprising a ribonucleoprotein complex (RNP), a DNA template, and at least one of an RNase inhibitor, N acetyl-L-cysteine (NAC), a histone deacetylase (HDAC) inhibitor, and/or an osmolarity-adjusting agent, wherein the RNP comprises a nuclease domain and a guide RNA, and wherein the 5′ and 3′ ends of the DNA template comprise nucleotide sequences that are homologous to genomic sequences flanking an insertion site in the genome of a cell. In some embodiments, the composition comprises at least two, at least three, or all four of an RNase inhibitor, N acetyl-L-cysteine (NAC), a histone deacetylase (HDAC) inhibitor, and/or an osmolarity-adjusting agent.

In some embodiments, the solution comprises at least: the N acetyl-L-cysteine (NAC) and the osmolarity-adjusting agent; the RNase inhibitor and the N acetyl-L-cysteine (NAC); the RNase inhibitor and the osmolarity-adjusting agent; the N acetyl-L-cysteine (NAC) and the histone deacetylase (HDAC) inhibitor; the osmolarity-adjusting agent and the histone deacetylase (HDAC) inhibitor; the RNase-inhibitor and the histone deacetylase (HDAC) inhibitor; the RNase inhibitor, the N acetyl-L-cysteine (NAC), and the histone deacetylase (HDAC) inhibitor; the N acetyl-L-cysteine (NAC), the osmolarity-adjusting agent, and the histone deacetylase (HDAC) inhibitor; the RNase inhibitor, the osmolarity-adjusting agent, and the histone deacetylase (HDAC) inhibitor; the RNase inhibitor, the N acetyl-L-cysteine (NAC), and the osmolarity-adjusting agent; or the RNase inhibitor, the N acetyl-L-cysteine (NAC), the osmolarity-adjusting agent, and the histone deacetylase (HDAC) inhibitor.

In some embodiments, the solution comprises at least one, two, three, or four of a final concentration of about 0.5 to 2 U/μl RNase inhibitor, a final concentration of about 1-10 mM NAC, a final concentration of about 100-400 mM sorbitol, and/or a final concentration of about 15.6 μM-4 mM sodium phenylbutyrate, 8 nM-200 nM quisinostat, or 3 nM-1 μM Panobinostat. In some embodiments, the solution comprises at least one, two, three, or four of a final concentration of about 1 U/μl RNase inhibitor, a final concentration of about 2.5 mM NAC, a final concentration of about 200 mM sorbitol, and/or a final concentration of about 1 mM sodium phenylbutyrate, 0.016 μM quisinostat, or 0.0375 μM Panobinostat.

In some aspects, additional compounds such as ATR inhibitors such as VE-822; PAPR-1 inhibitors, such as AG14361; Pole inhibitors such as ART-558; a CHK inhibitors such as AZD7762; and/or DNA-Pk inhibitors such as KU0600648 and NU7026, can also be used alone or in combination with the inhibitors and agents described here.

In one aspect, provided herein are composition comprising a solution comprising at least one of an ATR inhibitor such as VE-822; a PAPR-1 inhibitor, such as AG14361; a Pole inhibitor such as ART-558; a CHK inhibitor such as AZD7762; and/or a DNA-Pk inhibitor such as KU0600648 or NU7026, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

In some aspects, provided herein are methods of editing a cell, comprising: providing a solution comprising at least one of an ATR inhibitor such as VE-822; a PAPR-1 inhibitor, such as AG14361; a Pole inhibitor such as ART-558; a CHK inhibitor such as AZD7762; and/or a DNA-Pk inhibitor such as KU0600648 or NU7026, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

### Homologous Recombination or DNA Repair Modulation Proteins

A cell provided herein can also comprise an exogenous heterologous homologous recombination or DNA repair modulation protein or gene encoding the same. Such proteins and/or genes can increase the efficiency of the non-viral engineering, resulting in increased target gene expression (e.g., increased heterologous gene expression such as a priming receptor and/or CAR). In some embodiments, the exogenous homologous recombination or DNA repair modulation protein is SWSAP1, a dominant negative KU80, or an AcrIIA8-CDT1 fusion protein

The sequences of the SWSAP1, Dominant Negative KU80, and AcrIIA8_CDT1 fusion protein are provided below:

SWSAP1 sequence:
(SEQ ID NO: 125)
MGSGPAAGPPLLLLGTPGSGKTALLFAAALEAAGEGQGPVLFLTRRPLQ
SMPRGTGTTLDPMRLQKIRFQYPPSTRELFRLLCSAHEAPGPAPSLLLL
DGLEEYLAEDPEPQEAAYLIALLLDTAAHFSHRLGPGRDCGLMVALQTQ
EEAGSGDVLHLALLQRYFPAQCWLQPDAPGPGEHGLRACLEPGGLGPRT
EWWVTFRSDGEMMIAPWPTQAGDPSSGKGSSSGGQP
Dominant Negative KU80 sequence:
(SEQ ID NO: 126)
MGSGQLNAVDALIDSMSLAKKDEKTDTLEDLFPTTKIPNPRFQRLFQCL
LHRALHPREPLPPIQQHIWNMLNPPAEVTTKSQIPLSKIKTLFPLIEAK
KKDQVTAQEIFQDNHEDGPTAKKLKTEQGGAHFSVSSLAEGSVTSVGSV
NPAENFRVLVKQKKASFEEASNQLINHIEQFLDTNETPYFMKSIDCIRA
FREEAIKFSEEQRFNNFLKALQEKVEIKQLNHFWEIVVQDGITLITKEE
ASGSSVTAEEAKKFLAPKDKPSGDTAAVFEEGGDVDDLLDMI
AcrIIA8_CDT1 fusion protein sequence:
(SEQ ID NO: 127)
MGSGSIFTDMIPAELLINEYKKGQSGAKHDNYVSVGRIMVAIYKNNSFK
NTGTVKYQDSTHSGITMSKVFIDGKEYRIDIDTQHYEVQDFDTSGRQTT
LILKRIDLYGGSGSGSPSPARPALRAPASATSGSRKRARPPAAPGRDQA
RPPARRRLRLSVDEVSSPSTPEAPDIPACPSPGQKIKKSTPAAGQPPHL
TSAQDQDTI.

In some embodiments, the SWSAP1 protein comprises the sequence as set forth in SEQ ID NO: 125; the dominant negative KU80 protein comprises the sequence as set forth in SEQ ID NO: 126, or the AcrIIA8-CDT1 fusion protein comprises the sequence as set forth in SEQ ID NO: 127.

Such exogenous heterologous homologous recombination or DNA repair modulation proteins can be encoded on an episomal plasmid for transient expression. Any suitable episomal plasmid can be used to encode the exogenous heterologous homologous recombination or DNA repair modulation proteins. The episomal plasmid can be delivered alongside the target transgene insertion cassette DNA and Cas9 RNP. In some cases, the episomal plasmid is non-integrating and non-replicative. Exemplary episomal vectors for gene expression in mammalian cells are provided in Van Craenenbroeck K, et al, Eur. J. Biochem. 267:5665-5678 (200); and Stavrou, E. F., et al. Episomal vectors based on S/MAR and the β-globin Replicator, encoding a synthetic transcriptional activator, mediate efficient γ-globin activation in haematopoietic cells. Sci Rep 9, 19765 (2019), both of which are hereby incorporated by reference in their entirety.

The exogenous heterologous homologous recombination or DNA repair modulation proteins can also be delivered to the cell as mRNA or protein using an electroporation protocol with in vitro transcription or protein expression techniques known in the art. Such methods achieve the same goal of delivering DNA repair promoting elements to the cell and are equally compatible with electroporation. For mRNA based delivery, the DNA used to synthesize the exogenous heterologous homologous recombination or DNA repair modulation proteins would include the standard elements required for in vitro transcription such as a T7 promoter and Kozak sequence. The mRNA would be expressed from the DNA and electroporated into the target cell using an electroporation protocol described herein.

For protein based delivery, the exogenous heterologous homologous recombination or DNA repair modulation gene can be cloned into a standard expression vector (for example a pET vector) which includes elements needed for protein expression. The protein can then be delivered into the target cell using

In another aspect, provided herein is a polypeptide comprising an AcrIIA8 peptide fused to a CDT1 peptide. In one embodiment, the polypeptide comprises the sequence as set forth in SEQ ID NO: 127.

In one aspect, provided herein are methods of editing a cell, comprising: providing a cell comprising an exogenous homologous recombination or DNA repair modulation protein; providing a solution comprising a ribonucleoprotein complex (RNP), a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

### Methods of Non-Viral Gene Editing

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

In some aspects, provided herein are methods of editing a cell, comprising providing a solution comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising providing a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising providing a solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising providing a solution comprising a ribonucleoprotein complex (RNP), a DNA template, and at least one of an RNase inhibitor, N acetyl-L-cysteine (NAC), a histone deacetylase (HDAC) inhibitor, and/or an osmolarity-adjusting agent, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

The cell to be edited can be incubated with at least one of any RNase inhibitor, N acetyl-L-cysteine (NAC), histone deacetylase (HDAC) inhibitor, and/or an osmolarity-adjusting agent disclosed herein prior to contacting the cell with a ribonucleoprotein complex (RNP) and a DNA template. The cell can then be edited to insert the DNA template into the genome of the cell by the non-viral gene editing methods described herein. The edited cell can also be incubated with at least one of any RNase inhibitor, N acetyl-L-cysteine (NAC), histone deacetylase (HDAC) inhibitor, and/or an osmolarity-adjusting agent disclosed herein after insertion of the DNA template into the genome of the cell. The RNase inhibitor, N acetyl-L-cysteine (NAC), histone deacetylase (HDAC) inhibitor, and/or osmolarity-adjusting agent and cell can be incubated together in a solution, such as an aqueous solution before, during, and/or after the non-viral editing process. In some embodiments, the RNase inhibitor, N acetyl-L-cysteine (NAC), histone deacetylase (HDAC) inhibitor, and/or osmolarity-adjusting agent and cell are incubated together prior to the editing process. In some embodiments, the RNase inhibitor, N acetyl-L-cysteine (NAC), histone deacetylase (HDAC) inhibitor, and/or osmolarity-adjusting agent and cell are incubated together during the editing process. In some embodiments, the RNase inhibitor, N acetyl-L-cysteine (NAC), histone deacetylase (HDAC) inhibitor, and/or osmolarity-adjusting agent and cell are incubated together after the editing process.

In some aspects, provided herein are methods of editing a cell, comprising: providing a cell comprising an exogenous homologous recombination or DNA repair modulation protein; providing a solution comprising a ribonucleoprotein complex (RNP), a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising: providing solution comprising a histone deacetylase (HDAC) inhibitor comprising at least one of sodium phenylbutyrate, quisinostat, or panobinostat, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising: providing a cell with a solution comprising a histone deacetylase (HDAC) inhibitor comprising at least one of sodium phenylbutyrate, quisinostat, or Panobinostat; contacting the cell with a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell. In some embodiment, the method further comprises contacting (e.g., incubating) the edited cell with a solution comprising a histone deacetylase (HDAC) inhibitor comprising at least one of sodium phenylbutyrate, quisinostat, or Panobinostat.

In some aspects, provided herein are methods of editing a cell, comprising: providing a cell comprising an exogenous homologous recombination or DNA repair modulation protein; providing a solution comprising a ribonucleoprotein complex (RNP), a DNA template, and at least one of an RNase inhibitor, N acetyl-L-cysteine (NAC), an osmolarity-adjusting agent or a histone deacetylase (HDAC) inhibitor, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some aspects, provided herein are methods of editing a cell, comprising: providing a solution comprising a ribonucleoprotein complex (RNP), a DNA template, and at least one of an RNase inhibitor, N acetyl-L-cysteine (NAC), an osmolarity-adjusting agent or or a histone deacetylase (HDAC) inhibitor, wherein the RNP comprises a nuclease domain and a guide RNA, and wherein the 5′ and 3′ ends of the DNA template comprise nucleotide sequences that are homologous to genomic sequences flanking an insertion site in the genome of the cell; and editing the cell via insertion of the DNA template into the insertion site in the genome of the cell.

In some embodiments, the solution comprises at least: the N acetyl-L-cysteine (NAC) and the osmolarity-adjusting agent; the RNase inhibitor and the N acetyl-L-cysteine (NAC); the RNase inhibitor and the osmolarity-adjusting agent; the N acetyl-L-cysteine (NAC) and the histone deacetylase (HDAC) inhibitor; the osmolarity-adjusting agent and the histone deacetylase (HDAC) inhibitor; the RNase-inhibitor and the histone deacetylase (HDAC) inhibitor; the RNase inhibitor, the N acetyl-L-cysteine (NAC), and the histone deacetylase (HDAC) inhibitor; the N acetyl-L-cysteine (NAC), the osmolarity-adjusting agent, and the histone deacetylase (HDAC) inhibitor; the RNase inhibitor, the osmolarity-adjusting agent, and the histone deacetylase (HDAC) inhibitor; the RNase inhibitor, the N acetyl-L-cysteine (NAC), and the osmolarity-adjusting agent or the RNase inhibitor, the N acetyl-L-cysteine (NAC), the osmolarity-adjusting agent, and the histone deacetylase (HDAC) inhibitor.

In some embodiments, the insertion of the DNA template into the genome of the cell is increased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution. In some embodiments, the insertion of the DNA template into the genome of the cell is increased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution. In some embodiments, the solution increases expansion of the edited cell or yield of the edited cell as compared to a control solution that does not comprise the at least one of an RNase inhibitor, N acetyl-L-cysteine (NAC), an osmolarity-adjusting agent, or a histone deacetylase (HDAC) inhibitor. In some embodiments, the expansion or yield of the edited cell is increased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution. In some embodiments, the expansion or yield of the edited cell is increased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution.

In some embodiments, the solution comprising at least one of an RNase inhibitor, N acetyl-L-cysteine (NAC), an osmolarity-adjusting agent, or a histone deacetylase (HDAC) inhibitor decreases death of the cell during the non-viral introduction of the RNP complex and DNA template into the cell as compared to a control solution that does not comprise the at least one of an RNase inhibitor, N acetyl-L-cysteine (NAC), an osmolarity-adjusting agent, or a histone deacetylase (HDAC) inhibitor. In some embodiments, the death of the cell is decreased by 2%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 300% or more as compared to the control solution. In some embodiments, the death of the cell is decreased by at least 0.25-fold, 0.5-fold, 0.75-fold, 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, or 4-fold, or more, as compared to the control solution.

In some embodiments, the edited cells are incubated with the HDAC inhibitor during and/or after electroporation. In other embodiments, the cells to be edited are incubated with a HDAC inhibitor solution (e.g., sodium phenylbutyrate) for about 2 days prior to non-virally introducing the RNP complex and DNA template into the cell. In some embodiments, the HDAC inhibitor pre-electroporation solution comprises sodium phenylbutyrate. In some embodiments, the HDAC inhibitor pre-electroporation solution comprises a final concentration of about 0.01 mM, 0.05 mM, 0.1 mM, 0.10 mM, 0.20 mM, 0.30 mM, 0.40 mM, 0.50 mM, 0.60 mM, 0.70 mM, 0.80 mM, 0.90 mM, or 1 mM sodium phenylbutyrate.

In some aspects, the methods provided herein are for editing an immune cell, optionally a primary immune cell or a primary human immune cell.

In some embodiments, the cell is a mammalian cell, a human cell, a hematopoietic cell, an immune cell, a primary immune cell, or a primary human immune cell. In some embodiments, the immune cell is an autologous immune cell. In some embodiments, the immune cell is an allogeneic immune cell. In some embodiments, the immune cell is a natural killer (NK) cell, a T cell, a CD8+ T cell, a CD4+ T cell, a primary T cell, or a T cell progenitor cell.

In some aspects, the RNP-DNA template is non-virally introduced into to the cell. Non-viral insertion of the RNP-DNA template can be accomplished via chemical or electrical methods. For example, electroporation can be used to introduce the DNA template into the cell.

In some embodiments, the non-viral introduction of the RNP-DNA template is electroporation. Any appropriate electroporation device can be used, including, but not limited, to the CTS Xenon, the Nucleofector, the 4D-Nucleofector, the Neon NxT, the CliniMACS, or the MaxCyte Flow. Electroporation machines are commercially available from a variety of vendors such as Invitrogen, BioRad, Thermo Fisher, Lonza, Gibco, Miltenyi Biotec, and Mirus, among others. In some embodiments, the electroporation device is the CTS Xenon, the Nucleofector, or the 4D-Nucleofector.

In some embodiments, the electroporation comprises at least one cycle. In some embodiments, the electroporation comprises more than one cycle (e.g., 2, 3, 4, 5, 6, 7, 8, 9, 10 cycles, or 4-10 cycles). A cycle can be one or more electric pulse of a defined pulse time length and voltage with a defined time between the pulses.

In some embodiments, the one or more electroporation cycle(s) comprises 1-8, 1-2, 1-4, 1-6, 2-4, 2-8, 4-6, 4-8 electric pulses, or 1, 2, 3, 4, 5, or 6 electric pulses. In some embodiments, the one or more electroporation cycle(s) comprises 4 electric pulses. In some embodiments, the one or more electroporation cycle(s) comprises 5 electric pulses.

In some embodiments, one or more cycles or pulses of the electroporation are independently carried out at 500-2500 volts (e.g., 500 volts, 600 volts, 700 volts, 800 volts, 900 volts, 1000 volts, 1100 volts, 1200 volts, 1300 volts, 1400 volts, 1500 volts, 1600 volts, 1700 volts, 1800 volts, 1900 volts, 2000 volts, 2100 volts, 2200 volts, 2300 volts, 2400 volts, 500-1000 volts, 100-1500 volts, 1500-2000 volts, 2000-2100 volts, 2100-2200 volts, 2200-2300 volts, or 2300-2400 volts, or any number in between). In some embodiments, the one or more cycle(s) of the electroporation are carried out at 2300 volts. In some embodiments, the one or more pulse(s) are carried out at 2300 volts. In some embodiments, the one or more pulses in each cycle can be carried out at different voltages independently of each other. In some embodiments, the one or more pulses in each cycle can all be carried out at the same voltage (e.g., 2300 volts). In some embodiments, the one or more pulses in each cycle can all be carried out at different voltages (e.g., a first pulse of any voltage between 500-2500 volts and a second pulse of any voltage between 500-2500 volts that is different than the first pulse).

In some embodiments, one or more pulses of the one or more cycles of electroporation are independently carried out using a pulse time duration of 1-5, 1, 1.5, 2, 2.5, 3, 3.5, 4, 4.5, or 5 ms. In some embodiments, one or more pulses of the one or more cycles of electroporation are independently carried out using a pulse time duration of 1-30 ms, 1 ms, 2 ms, 3 ms, 4 ms, 5 ms, 6 ms, 7 ms, 8 ms, 9 ms, 10 ms, 11 ms, 12 ms, 13 ms, 14 ms, 15 ms, 16 ms, 17 ms, 18 ms, 19 ms, 20 ms, 21 ms, 22 ms, 23 ms, 24 ms, 25 ms, 26 ms, 27 ms, 28 ms, 29 ms, or 30 ms. In certain embodiments, one or more pulses of the one or more cycles of the electroporation are independently carried out using a pulse duration of 3 ms.

In some embodiments, some or all cycles of the electroporation are independently carried out using 1-8, 1, 2, 3, 4, 5, 6, 7, or 8 pulses.

In some embodiments, the one or more cycles of the electroporation are independently carried out using a pulse interval of 100-1000, 100, 150, 200, 250, 300, 350, 400, 450, 500, 550, 600, 650, 700, 750, 800, 850, 900, 950, or 100 ms between the electrical pulses. In some embodiments, the one or more cycles of the electroporation are independently carried out using a pulse interval of 500 ms.

In some embodiments, the electroporation comprises one cycle carried out using the settings of: 2200 volts, 3.0 ms pulse duration, four pulses, and 500 ms pulse interval.

In some embodiments, the electroporation comprises one cycle carried out using the settings of: 2300 volts, 3.0 ms pulse duration, four pulses, and 500 ms pulse interval.

In some embodiments, the electroporation comprises one cycle carried out using the settings of: 2300 volts, 3.0 ms pulse duration, five pulses, and 500 ms pulse interval.

In some embodiments, the amount of DNA template used in the one or more cycles of electroporation is about 1 μg, 2 μg, 5 μg, 10 μg, 15 μg, 20 μg, 25 μg, 30 μg, 35 μg, 40 μg, 45 μg, or 50 μg of DNA or between about 1-10 μg, 10-20 μg, 20-30 μg, 30-40 μg, or 40-50 μg of DNA.

In some embodiments, the electroporation comprises 5 pulses and a lower amount of DNA template as compared to an electroporation comprising 4 pulses.

In some embodiments, the electroporation comprises the EH115 pulse code.

### Insertion Sites

Methods for editing the genome of an immune cell, specifically, include a method of editing the genome of a human T cell comprise inserting a nucleic acid sequence or construct into a target region in exon 1 of the TCR-α subunit (TRAC) gene in a T cell. In some embodiments, the target region is in exon 1 of the constant domain of TRAC gene. In other embodiments, the target region is in exon 1, exon 2 or exon 3, prior to the start of the sequence encoding the TCR-α transmembrane domain. In some embodiments, the nucleic acid sequence or construct encodes a heterologous protein, such as but not limited to, a priming receptor and/or a chimeric antigen receptor (CAR).

Methods for editing the genome of an immune cell also include a method of editing the genome of a human T cell comprise inserting a nucleic acid sequence or construct into a target region in exon 1 of a TCR-subunit (TRBC) gene in the human T cell. In some embodiments, the target region is in exon 1 of the TRBC1 or TRBC2 gene.

Methods for editing the genome of an immune cell, specifically, include a method of editing the genome of a human T cell comprise inserting a nucleic acid sequence or construct into a target region of a genomic safe harbor (GSH).

Gene editing therapies include, for example, viral vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc., These challenges can be addressed, in part, through the identification and use of safe harbor loci or safe harbor sites (SHS), which are sites in which genes or genetic elements can be incorporated without disruption to expression or regulation of adjacent genes.

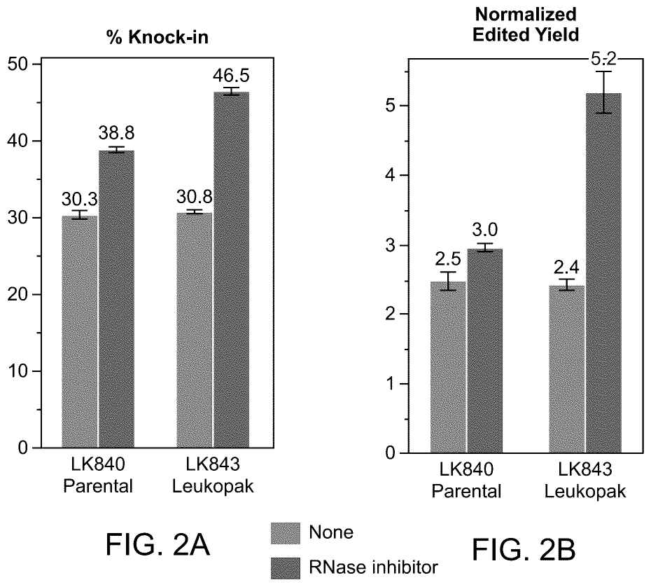

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See FIG. 2 from Sadelain, M., et al. (2012). Safe harbours for the integration of new DNA in the human genome. Nature reviews Cancer, 12 (1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. An exemplary AAVS1 target gRNA and target sequence are provided below:

•AAVS1-gRNA sequence:
(SEQ ID NO: 128)
ggggccactagggacaggatGTTTTAGAGCTAGAAATAGCAAGTTAAAA
TAAGGCTAGTCCGTTATCAACTTGAAAAAGTGGCACCGAGTCGGTGCTT
TTTTT
•AAVS1 target sequence:
(SEQ ID NO: 129)
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

Additional safe harbor sites and the accompanying sgRNA sequences for targeting those SHSs are provided in Table 1.

TABLE 1
SGRNA SEQUENCES USED FOR TARGETING SAFE HARBOR SITES

| | | | | Integra- |
|---|---|---|---|---|
| | | sgRNA start | sgRNA | tion |
| sgRNA ID | sgRNA Sequence | coor GRCH38 | Target Loci | Site |
| sgRNA_1 | GCACCTGAATACCACGCCTG (SEQ ID NO: 1) | chr16: 88811818 | APRT | APRT |
| sgRNA_2 | CGCCTGCGATGTAGTCGATG (SEQ ID NO: 2) | chr16: 88811551 | APRT | APRT |
| sgRNA_3 | CAGGACGGGCGAGATGTCCC (SEQ ID NO: 3) | chr16: 88811640 | APRT | APRT |
| sgRNA_4 | CTGAATCTTTGGAGTACCTG (SEQ ID NO: 4) | chr15: 44715425 | B2M | B2M |
| sgRNA_5 | GGCCACGGAGCGAGACATCT (SEQ ID NO: 5) | chr15: 44711550 | B2M | B2M |
| sgRNA_6 | AAGTCAACTTCAATGTCGGA (SEQ ID NO: 6) | chr15: 44715515 | B2M | B2M |
| sgRNA_7 | GCTTGGAGGCCTGATCAGCG (SEQ ID NO: 7) | chr19: 36141111 | CAPNS1 | CAPNS1 |
| sgRNA_8 | CTTATCTCTTCGCAGCGAGG (SEQ ID NO: 8) | chr19: 36142301 | CAPNS1 | CAPNS1 |
| sgRNA_9 | CACACATTACTCCAACATTG (SEQ ID NO: 9) | chr19: 36142676 | CAPNS1 | CAPNS1 |
| sgRNA_10 | TTCCGCAAAATAGAGCCCCA (SEQ ID NO: 10) | chr3: 105746019 | CBLB | CBLB |
| sgRNA_11 | TGCACAGAACTATCGTACCA (SEQ ID NO: 11) | chr3: 105751622 | CBLB | CBLB |
| sgRNA_12 | GCAATAAGACTCTTTAAAGA (SEQ ID NO: 12) | chr3: 105853470 | CBLB | CBLB |
| sgRNA_13 | CAAAGAGATTACGAATGCCT (SEQ ID NO: 13) | chr1: 116754658 | CD2 | CD2 |
| sgRNA_14 | CAAGGCACCCCAGGTTTCCA (SEQ ID NO: 14) | chr1: 116754663 | CD2 | CD2 |
| sgRNA_15 | TTACGAATGCCTTGGAAACC (SEQ ID NO: 15) | chr1: 116754666 | CD2 | CD2 |
| sgRNA_16 | CAGAGACGCATCTGACCCTC (SEQ ID NO: 16) | chr11: 118315540 | CD3E | CD3E |
| sgRNA_17 | CATGCAGTTCTCACACACTG (SEQ ID NO: 17) | chr11: 118313715 | CD3E | CD3E |
| sgRNA_18 | GTGTGAGAACTGCATGGAGA (SEQ ID NO: 18) | chr11: 118313715 | CD3E | CD3E |
| sgRNA_19 | TCTCATTTCAGGAAACCACT (SEQ ID NO: 19) | chr11: 118349748 | CD3G | CD3G |
| sgRNA_20 | AGTCATACACCTTAACCAAG (SEQ ID NO: 20) | chr11: 118349754 | CD3G | CD3G |
| sgRNA_21 | TTCAAGGAAACCAGTTGAGG (SEQ ID NO: 21) | chr11: 118352458 | CD3G | CD3G |
| sgRNA_22 | GAGCCTTGCCTGGAAATCTG (SEQ ID NO: 22) | chr11: 61118177 | CD5 | CD5 |
| sgRNA_23 | AAGCGTCAAAAGTCTGCCAG (SEQ ID NO: 23) | chr11: 61118324 | CD5 | CD5 |
| sgRNA_24 | CGTTCCAACTCGAAGTGCCA (SEQ ID NO: 24) | chr11: 61118121 | CD5 | CD5 |
| sgRNA_25 | GAGCGACTGGGACACGGTGA (SEQ ID NO: 25) | chr9: 136866246 | EDF1 | EDF1 |
| sgRNA_26 | GCTGCGCAAGAAGGGCCCTA (SEQ ID NO: 26) | chr9: 136866211 | EDF1 | EDF1 |
| sgRNA_27 | TTGTTCTGGCCAGCAGCCCC (SEQ ID NO: 27) | chr9: 136863433 | EDF1 | EDF1 |
| sgRNA_28 | CTTCCAGAGCCACATCATCG (SEQ ID NO: 28) | chr19: 48965791 | FTL | FTL |
| sgRNA_29 | GGGACTCACCAGAGAGAGGT (SEQ ID NO: 29) | chr19: 48965601 | FTL | FTL |
| sgRNA_30 | CGGTCGAAATAGAAGCCCTA (SEQ ID NO: 30) | chr19: 48965770 | FTL | FTL |
| sgRNA_31 | AAAAGGATATTGTGCAACTG (SEQ ID NO: 31) | chr10: 87933015 | PTEN | PTEN |
| sgRNA_32 | TGTGCATATTTATTACATCG (SEQ ID NO: 32) | chr10: 87933183 | PTEN | PTEN |
| sgRNA_33 | TTTGTGAAGATCTTGACCAA (SEQ ID NO: 33) | chr10: 87933087 | PTEN | PTEN |
| sgRNA_34 | TGTCATGCTGAACCGCATTG (SEQ ID NO: 34) | chr18: 12830972 | PTPN2 | PTPN2 |
| sgRNA_35 | CCACTCTATGAGGATAGTCA (SEQ ID NO: 35) | chr18: 12859219 | PTPN2 | PTPN2 |
| sgRNA_36 | TTGACATAGAAGAGGCACAA (SEQ ID NO: 36) | chr18: 12836828 | PTPN2 | PTPN2 |
| sgRNA_37 | GAGTACTACACTCAGCAGCA (SEQ ID NO: 37) | chr12: 6952098 | PTPN6 | PTPN6 |
| sgRNA_38 | TCACGCACAAGAAACGTCCA (SEQ ID NO: 38) | chr12: 6954872 | PTPNG | PTPN6 |
| sgRNA_39 | AGGTCTCGGTGAAACCACCT (SEQ ID NO: 39) | chr12: 6951610 | PTPN6 | PTPN6 |
| sgRNA_40 | AGCATTATCCAAAGAGTCCG (SEQ ID NO: 40) | chr1: 198696873 | PTPRC | PTPRC |
| sgRNA_41 | ATATTAATTCTTACCAGTGG (SEQ ID NO: 41) | chr1: 198692370 | PTPRC | PTPRC |
| sgRNA_42 | AGCTTTAAATCAAGGTTCAT (SEQ ID NO: 42) | chr1: 198756176 | PTPRC | PTPRC |
| sgRNA_43 | ATCCCGAGCCCTAAGGTGCA (SEQ ID NO: 43) | chr11: 67436325 | PTPRCAP | PTPRCAP |
| sgRNA_44 | GGCAGCGCGGAGGACAGCGT (SEQ ID NO: 44) | chr11: 67436285 | PTPRCAP | PTPRCAP |
| sgRNA_45 | CTCAGGGGGCTACTACCACC (SEQ ID NO: 45) | chr11: 67436170 | PTPRCAP | PTPRCAP |
| sgRNA_46 | GTCACCGACGAGACCAGAAG (SEQ ID NO: 46) | chr5: 82277810 | RPS23 | RPS23 |
| sgRNA_47 | GTCGTGGACTTCGTACTGCT (SEQ ID NO: 47) | chr5: 82277843 | RPS23 | RPS23 |
| sgRNA_48 | TAATTTTTAGGCAAGTGTCG (SEQ ID NO: 48) | chr5: 82277860 | RPS23 | RPS23 |
| sgRNA_49 | TTAGCTGTTAGACTTGAATA (SEQ ID NO: 49) | chr14: 51993810 | RTRAF | RTRAF |
| sgRNA_50 | CGAGAGCCGTCAACTTGCGT (SEQ ID NO: 50) | chr14: 51989652 | RTRAF | RTRAF |
| sgRNA_51 | CGGCTTCAACTGCAAAGGTG (SEQ ID NO: 51) | chr14: 51989700 | RTRAF | RTRAF |
| sgRNA_52 | TATGAAAAAGCAGAGCGACT (SEQ ID NO: 52) | chr15: 43793025 | SERF2 | SERF2 |
| sgRNA_53 | TCTGGCGGGCGAGCTCACGC (SEQ ID NO: 53) | chr15: 43792989 | SERF2 | SERF2 |
| sgRNA_54 | CTCACGCTGGTTACCGCCTA (SEQ ID NO: 54) | chr15: 43792977 | SERF2 | SERF2 |
| sgRNA_55 | AAAGATTACGAACTTCCCTG (SEQ ID NO: 55) | chr12: 46207559 | SLC38A1 | SLC38A1 |
| sgRNA_56 | GTTAAAAACAGACATGCCTA (SEQ ID NO: 56) | chr12: 46229232 | SLC38A1 | SLC38A1 |
| sgRNA_57 | ATGCCTAAGGAGGTTGTACC (SEQ ID NO: 57) | chr12: 46229246 | SLC38A1 | SLC38A1 |
| sgRNA_58 | CTCCAGGTATCCCATCGAAA (SEQ ID NO: 58) | chr18: 47869418 | SMAD2 | SMAD2 |
| sgRNA_59 | CACCAAATACGATAGATCAG (SEQ ID NO: 59) | chr18: 47870532 | SMAD2 | SMAD2 |
| sgRNA_60 | TGGCGGCGTGAATGGCAAGA (SEQ ID NO: 60) | chr18: 47896729 | SMAD2 | SMAD2 |
| sgRNA_61 | TAGGATGGTAGCACACAACC (SEQ ID NO: 61) | chr16: 11255478 | SOCS1 | SOCSI |
| sgRNA_62 | CAGCAGCAGAGCCCCGACGG (SEQ ID NO: 62) | chr16: 11255432 | SOCS1 | SOCS1 |
| sgRNA_63 | CGGCGTGCGAACGGAATGTG (SEQ ID NO: 63) | chr16: 11255296 | SOCS1 | SOCS1 |
| sgRNA_64 | TATAGACGCTGCCCGACGTC (SEQ ID NO: 64) | chr15: 40038895 | SRP14 | SRP14 |
| sgRNA_65 | TCCAAAGAAGGGTACTGTGG (SEQ ID NO: 65) | chr15: 40038368 | SRP14 | SRP14 |
| sgRNA_66 | ACAGTACCCTTCTTTGGAAT (SEQ ID NO: 66) | chr15: 40038358 | SRP14 | SRP14 |
| sgRNA_67 | GCGACGGGCGCATCTACGTG (SEQ ID NO: 67) | chr12: 120469572 | SRSF9 | SRSF9 |
| sgRNA_68 | CCCGACCTCCATAAGTCCTG (SEQ ID NO: 68) | chr12: 120465700 | SRSF9 | SRSF9 |
| sgRNA_69 | GGGGTCCTCGAAGCGCACGA (SEQ ID NO: 69) | chr12: 120469426 | SRSF9 | SRSF9 |
| sgRNA_70 | TGCTCTGTTTAGAAGATGAC (SEQ ID NO: 70) | chr5: 32591641 | SUB1 | SUB1 |
| sgRNA_71 | ATATTCTTTTCTAGTTAAAG (SEQ ID NO: 71) | chr5: 32591566 | SUB1 | SUB1 |
| sgRNA_72 | CCTGTAAAGAAACAAAAGAC (SEQ ID NO: 72) | chr5: 32591614 | SUB1 | SUB1 |
| sgRNA_73 | TGGAGAAAGACGTAACTTCG (SEQ ID NO: 73) | chr4: 105234315 | TET2 | TET2 |
| sgRNA_74 | TCTGCCCTGAGGTATGCGAT (SEQ ID NO: 74) | chr4: 105234747 | TET2 | TET2 |
| sgRNA_75 | ATTCCGCTTGGTGAAAACGA (SEQ ID NO: 75) | chr4: 105235656 | TET2 | TET2 |
| sgRNA_76 | CAGGCACAATAGAAACAACG (SEQ ID NO: 76) | chr3: 114295571 | TIGIT | TIGIT |
| sgRNA_77 | CCATTTGTAATGCTGACTTG (SEQ ID NO: 77) | chr3: 114295700 | TIGIT | TIGIT |
| sgRNA_78 | CTGGGTCACTTGTGCCGTGG (SEQ ID NO: 78) | chr3: 114295634 | TIGIT | TIGIT |
| sgRNA_79 | GTCAGGGTTCTGGATATCTG (SEQ ID NO: 79) | chr14: 22547508 | TRAC | TRAC |
| sgRNA_80 | TGGATTTAGAGTCTCTCAGC (SEQ ID NO: 80) | chr14: 22547541 | TRAC | TRAC |
| sgRNA_81 | CTGCGGCTGTGGTCCAGCTG (SEQ ID NO: 81) | chr14: 22550661 | TRAC | TRAC |
| sgRNA_82 | ACAAAACTGTGCTAGACATG (SEQ ID NO: 82) | chr14: 22547658 | TRAC | TRAC |
| sgRNA_83 | TTCTTCCCCAGCCCAGGTAA (SEQ ID NO: 83) | chr14: 22547778 | TRAC | TRAC |
| sgRNA_84 | CGTCATGAGCAGATTAAACC (SEQ ID NO: 84) | chr14: 22550625 | TRAC | TRAC |
| sgRNA_85 | GAGAGCGCCTGCGACCCGAG (SEQ ID NO: 85) | chr19: 58544980 | TRIM28 | TRIM28 |
| sgRNA_86 | CCAGCGGGTGAAGTACACCA (SEQ ID NO: 86) | chr19: 58544869 | TRIM28 | TRIM28 |
| sgRNA_87 | GGAGCGCTTTTCGCCGCCAG (SEQ ID NO: 87) | chr19: 58544839 | TRIM28 | TRIM28 |
| sgRNA_88 | TGAGGCCTGGACCTTATGCA (SEQ ID NO: 88) | chr10: 33134193 | chr10: | desert_1 |
| | | | 33130000- | (GS88) |
| | | | 33140000 | |
| sgRNA_89 | CCTGGTGGAGTGAACCATGA (SEQ ID NO: 89) | chr10: 33132917 | chr10: | desert_1 |
| | | | 33130000- | (GS89) |
| | | | 33140000 | |
| sgRNA_90 | CAAGCACTTAGGTTCCCCTG (SEQ ID NO: 90) | chr10: 33134633 | chr10: | desert_1 |
| | | | 33130000- | (GS90) |
| | | | 33140000 | |
| sgRNA_91 | GGTCTCCCTACAATTCAGCG (SEQ ID NO: 91) | chr10: 72294568 | chr10: | desert_2 |
| | | | 72290000- | (GS91) |
| | | | 72300000 | |
| sgRNA_92 | CACAGCGCGTGACTGCAATG (SEQ ID NO: 92) | chr10: 72298268 | chr10: | desert_2 |
| | | | 72290000- | (GS92) |
| | | | 72300000 | |
| sgRNA_93 | TCTGGGGCACCAATTCTAGG (SEQ ID NO: 93) | chr10: 72292786 | chr10: | desert_2 |
| | | | 72290000- | (GS93) |
| | | | 72300000 | |
| sgRNA_94 | GAGCCATGCTTGGCTTACGA (SEQ ID NO: 94) | chr11: 128342576 | chr11: | desert_3 |
| | | | 128340000- | (GS94) |
| | | | 128350000 | |
| sgRNA_95 | GTACAAGTACTTATCTCATG (SEQ ID NO: 95) | chr11: 128343592 | chr11: | desert_3 |
| | | | 128340000- | (GS95) |
| | | | 128350000 | |
| sgRNA_96 | GAGATAACAACATAACAACA (SEQ ID NO: 96) | chr11: 128347170 | chr11: | desert_3 |
| | | | 128340000- | (GS96) |
| | | | 128350000 | |
| sgRNA_97 | CATATTCCATAGTCTTTGGG (SEQ ID NO: 97) | chr11: 65425000 | chr11: | desert_4 |
| | | | 65425000- | (GS97) |
| | | | 65427000 | |
| | | | (NEAT1) | |
| sgRNA_98 | CTGCCCCTTAGCAACTTAGG (SEQ ID NO: 98) | chr11: 65425507 | chr11: | desert_4 |
| | | | 65425000- | (GS98) |
| | | | 65427000 | |
| | | | (NEAT1) | |
| sgRNA_99 | TGTTTAAAAATATGTTGACA (SEQ ID NO: 99) | chr11: 65426264 | chr11: | desert_4 |
| | | | 65425000- | (GS99) |
| | | | 65427000 | |
| | | | (NEAT1) | |
| sgRNA_100 | CCAGGAATGGAAACTCACGC (SEQ ID | chr15: 92830315 | chr15: | desert_5 |
| | NO: 100) | | 92830000- | (GS100) |
| | | | 92840000 | |
| sgRNA_101 | GAGGCCGCTGAATTAACCCG (SEQ ID | chr15: 92831850 | chr15: | desert_5 |
| | NO: 101) | | 92830000- | (GS101) |
| | | | 92840000 | |
| sgRNA_102 | ATACACGCACACTTGCAGAA (SEQ ID | chr15: 92831131 | chr15: | desert_5 |
| | NO: 102) | | 92830000- | (GS102) |
| | | | 92840000 | |
| sgRNA_103 | GAGCAGACAGAAACCCAGGG (SEQ ID | chr16: 11225670 | chr16: | desert_6 |
| | NO: 103) | | 11220000- | (GS103) |
| | | | 11230000 | |
| sgRNA_104 | TGAGTCTCCAAACAGAACAG (SEQ ID | chr16: 11226284 | chr16: | desert_6 |
| | NO: 104) | | 11220000- | (GS104) |
| | | | 11230000 | |
| sgRNA_105 | TAATATCACTGACTTCACGG (SEQ ID | chr16: 11225029 | chr16: | desert_6 |
| | NO: 105) | | 11220000- | (GS105) |
| | | | 11230000 | |
| sgRNA_106 | TACACACAATGTAAGCAGCA (SEQ ID | chr2: 87467461 | chr2: | desert_7 |
| | NO: 106) | | 87460000- | (GS106) |
| | | | 87470000 | |
| sgRNA_107 | GGGAGCTCAATTCGAAACCA (SEQ ID | chr2: 87468809 | chr2: | desert_7 |
| | NO: 107) | | 87460000- | (GS107) |
| | | | 87470000 | |
| sgRNA_108 | TTGGACAGGTGAGACAGTCG (SEQ ID | chr2: 87467001 | chr2: | desert_7 |
| | NO: 108) | | 87460000- | (GS108) |
| | | | 87470000 | |
| sgRNA_109 | AAGCTCACTCAGATAGTGTG (SEQ ID | chr3: 186511316 | chr3: | desert_8 |
| | NO: 109) | | 186510000- | (GS109) |
| | | | 186520000 | |
| sgRNA_110 | CAGGAGAACCACCTTACACG (SEQ ID | chr3: 186515260 | chr3: | desert_8 |
| | NO: 110) | | 186510000- | (GS110) |
| | | | 186520000 | |
| sgRNA_111 | GGACAGACCCTGATTCACAA (SEQ ID | chr3: 186519655 | chr3: | desert_8 |
| | NO: 111) | | 186510000- | (GS111) |
| | | | 186520000 | |
| sgRNA_112 | ACATGGCAGTCTATGAACAG (SEQ ID | chr3: 59451154 | chr3: | desert_9 |
| | NO: 112) | | 05945000- | (GS112) |
| | | | 59460000 | |
| sgRNA_113 | CCTATAGAGAGTACTACTTG (SEQ ID | chr3: 59456416 | chr3: | desert_9 |
| | NO: 113) | | 59450000- | (GS113) |
| | | | 59460000 | |
| sgRNA_114 | CCAACCGGGTCTTCATTACG (SEQ ID | chr3: 59457029 | chr3: | desert_9 |
| | NO: 114) | | 59450000- | (GS114) |
| | | | 59460000 | |
| sgRNA_115 | TCAAGCGTAGAGTTCCGAGT (SEQ ID | chr8: 127993006 | chr8: | desert_ |
| | NO: 115) | | 127980000- | 10 |
| | | | 128000000 | (GS115) |
| sgRNA_116 | TCATGCAATTATGGACCCAG (SEQ ID | chr8: 127994663 | chr8: | desert_ |
| | NO: 116) | | 127980000- | 10 |
| | | | 128000000 | (GS116) |
| sgRNA_117 | CGGGAAAGTGACTGGCCATG (SEQ ID | chr8: 127996766 | chr8: | desert_ |
| | NO: 117) | | 127980000- | 10 |
| | | | 128000000 | (GS117) |
| sgRNA_118 | TGAGATTGAAATCAAATCGG (SEQ ID | chr9: 7974159 | chr9: | desert_ |
| | NO: 118) | | 7970000- | 11 |
| | | | 7980000 | (GS118) |
| sgRNA_119 | TATGCAATATTCATCACGCG (SEQ ID | chr9: 7977914 | chr9: | desert_ |
| | NO: 119) | | 7970000- | 11 |
| | | | 7980000 | (GS119) |
| sgRNA_120 | AATGTGTTAAATCAAATGCA (SEQ ID | chr9: 7976895 | chr9: | desert_ |
| | NO: 120) | | 7970000- | 11 |
| | | | 7980000 | (GS120) |

In some embodiments, the safe harbor locus is at any one or more of the sgRNA target loci selected from: chr10:33130000-33140000, chr10:72290000-72300000, chr11:128340000-128350000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, chr9:7970000-7980000, APRT, B2M, CAPNS1, CBLB, CD2, CD3E, CD3G, CD5, EDF1, FTL, PTEN, PTPN2, PTPN6, PTPRC, PTPRCAP, RPS23, RTRAF, SERF2, SLC38A1, SMAD2, SOCS1, SRP14, SRSF9, SUB1, TET2, TIGIT, TRAC, and TRIM28.

In some embodiments, the target locus is selected from: chr10:33130000-33140000, chr10:72290000-72300000, chr11:128340000-128350000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, and chr9:7970000-7980000.

In some embodiments, the target locus is chr11:128340000-128350000 or chr15:92830000-92840000.

In some embodiments, the target locus is a gene selected from: APRT, B2M, CAPNS1, CBLB, CD2, CD3E, CD3G, CD5, EDF1, FTL, PTEN, PTPN2, PTPN6, PTPRC, PTPRCAP, RPS23, RTRAF, SERF2, SLC38A1, SMAD2, SOCS1, SRP14, SRSF9, SUB1, TET2, TIGIT, TRAC, and TRIM28.

In some embodiments, the safe harbor locus is the GS94 or GS102 integration site in Table 1.

In some embodiments, the safe harbor loci of the present disclosure are useful for the insertion of a sequence encoding a transgene. In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 KB, about 525 kB, about 550 kB away from the safe harbor locus (integration site).

In some embodiments, the present disclosure contemplates inserts that comprise one or more transgenes. The transgene can encode a therapeutic protein, an antibody, a peptide, a suicide gene, an apoptosis gene or any other gene of interest. The safe harbor loci identified using the method described herein allow for transgene integration that results in, for example, enhanced therapeutic properties. These enhanced therapeutic properties, as used herein, refer to an enhanced therapeutic property of a cell when compared to a typical immune cell of the same normal cell type. For example, an NK cell having “enhanced therapeutic properties” has an enhanced, improved, and/or increased treatment outcome when compared to a typical, unmodified and/or naturally occurring NK cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the safe harbor site. In some embodiments, the insert size comprises at least about 100, 200, 300, 400 or 500 nucleotides (basepairs). In some embodiments, the insert size comprises about 500 nucleotides (basepairs). In some embodiments, the insert size comprises up to 0.5, 0.6, 0.7, 0.8, 0.9, 1, 2, 3, 4, 5, 6, 7, 8, 9, 10, 11, 12, 13, 14, 15, 16, 17, 18, 19, 20 kbp (kilo basepairs) or the sizes in between. In some embodiments, the insert size is greater than 0.5, 0.6, 0.7, 0.8, 0.9, 1, 2, 3, 4, 5, 6, 7, 8, 9, 10, 11, 12, 13, 14, 15, 16, 17, 18, 19, 20 kbp or the sizes in between. In some embodiments, the insert size is within the range of 3-15 kbp or is any number in that range. In some embodiments, the insert size is within the range of 1.5-8.3 kbp or is any number in that range. In some embodiments, the insert size is within the range of 1.5-13 kbp or is any number in that range. In some embodiments, the insert size is within the range of at least 1.5-15 kbp or is any number in that range. In some embodiments, the insert size is within the range of 0.5-20 kbp or is any number in that range. In some embodiments, the insert size is 0.5-10, 0.6-10, 0.7-10, 0.8-10, 0.9-10, 1-10, 2-10, 3-10, 4-10, 5-10, 6-10, 7-10, 8-10, 9-10 kbp. In some embodiments, the insert size is 0.5-11, 0.6-11, 0.7-11, 0.8-11, 0.9-11, 1-11, 2-11, 3-11, 4-11, 5-11, 6-11, 7-11, 8-11, 9-11, or 10-11 kbp. In some embodiments, the insert size is 0.5-12, 0.6-12, 0.7-12, 0.8-12, 0.9-12, 1-12, 2-12, 3-12, 4-12, 5-12, 6-12, 7-12, 8-12, 9-12, 10-12, or 11-12 kbp. In some embodiments, the insert size is 0.5-13, 0.6-13, 0.7-13, 0.8-13, 0.9-13, 1-13, 2-13, 3-13, 4-13, 5-13, 6-13, 7-13, 8-13, 9-13, 10-13, 11-13, or 12-13 kbp. In some embodiments, the insert size is 0.5-14, 0.6-14, 0.7-14, 0.8-14, 0.9-14, 1-14, 2-14, 3-14, 4-14, 5-14, 6-14, 7-14, 8-14, 9-14, 10-14, 11-14, 12-14 or 13-14 kbp. In some embodiments, the insert size is 0.5-15, 0.6-15, 0.7-15, 0.8-15, 0.9-15, 1-15, 2-15, 3-15, 4-15, 5-15, 6-15, 7-15, 8-15, 9-15, 10-15, 11-15, 12-15, 13-15, or 14-15 kbp. In some embodiments, the insert size is 0.5-16, 0.6-16, 0.7-16, 0.8-16, 0.9-16, 1-16, 2-16, 3-16, 4-16, 5-16, 6-16, 7-16, 8-16, 9-16, 10-16, 11-16, 12-16, 13-16, 14-16 or 15-16 kbp. In some embodiments, the insert size is 0.5-17, 0.6-17, 0.7-17, 0.8-17, 0.9-17, 1-17, 2-17, 3-17, 4-17, 5-17, 6-17, 7-17, 8-17, 9-17, 10-17, 11-17, 12-17, 13-17, or 14-17, 15-17 or 16-17 kbp. In some embodiments, the insert size is 0.5-18, 0.6-18, 0.7-18, 0.8-18, 0.9-18, 1-18, 2-18, 3-18, 4-18, 5-18, 6-18, 7-18, 8-18, 9-18, 10-18, 11-18, 12-18, 13-18, 14-18, 15-18, 16-18 or 17-18 kbp. In some embodiments, the insert size is 0.5-19, 0.6-19, 0.7-19, 0.8-19, 0.9-19, 1-19, 2-19, 3-19, 4-19, 5-19, 6-19, 7-19, 8-19, 9-19, 10-19, 11-19, 12-19, 13-19, 14-19, 15-19, 16-19, 17-19, or 18-19 kbp. In some embodiments, the insert size is 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 kbp.

The inserts of the present disclosure refer to nucleic acid molecules or polynucleotide inserted at a safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), syn-notch, CARs, mAbs, etc. In some embodiments, the inserts are RNAi molecules, including, but not limited to, miRNAs, siRNA, shRNAs, etc. In some embodiments, the insert is a human sequence. In some embodiments, the insert is chimeric. In some embodiments, the insert is a multi-gene/multi-module therapeutic cassette. A multi-gene/multi-module therapeutic cassette refers to an insert or cassette having one or more than one receptor (e.g., synthetic receptors), other exogenous protein coding sequences, non-coding RNAs, transcriptional regulatory elements, and/or insulator sequences, etc.

Various cell types are contemplated as having the safe harbor sites in the present disclosure. A cell comprising a safe harbor site and/or a cell comprising an insert at a safe harbor site as described in the present disclosure can be referred to as an engineered cell. The cells can include, but are not limited to, eukaryotic cells, prokaryotic cells, animal cells, plant cells, fungal cells and the like. Optionally, the cell is a mammalian cell, for example, a human cell. In some embodiments, that engineered cell is a stem cell, a human cell, a primary cell, an hematopoietic cell, an adaptive immune cell, an innate immune cell, a T cell or a T cell progenitor. Non-limiting examples of immune cells that are contemplated in the present disclosure include T cell, B cell, natural killer (NK) cell, NKT/INKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

The methods for integrating the inserts at the safe harbor sites can be non-viral delivery techniques.

In some embodiments, the nucleic acid sequence is inserted into the genome of the cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9. In some embodiments, the non-viral delivery method comprises electroporation.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

The engineered cell can retain its undifferentiated state after insertion of the transgenes. In some embodiments, the engineered cell is undifferentiated. In some embodiments, the engineered cell is undifferentiated after insert of the transgene. In some embodiments, the engineered cell is CD45RA⁺ and CCR7⁺ after insertion of the transgene. In some embodiments, the engineered cell is CD45RA⁺CCR7⁺CD27⁺ after insertion of the transgene.

### Crispr-Cas Gene Editing

One effective example of gene editing is the Crisp-Cas approach (e.g. Crispr-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540 (7631), 144-149, the relevant disclosures of which are herein incorporated in their entirety).

Methods for conducing gene editing using CRISPR-Cas approaches are known to those of ordinary skill in the art. (See, for example, U.S. Ser. Nos. 16/312,676, 15/303,722, and 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example U.S. application Ser. No. 15/715,068, the disclosures of which are herein incorporated by reference in their entirety.

### Nucleic Acids and Vectors

In some embodiments, the present disclosure contemplates nucleic acid inserts that comprise one or more transgenes. The transgene can encode a therapeutic protein, an antibody, a peptide, a suicide gene, an apoptosis gene or any other gene of interest. In some embodiments, the transgene encodes a priming receptor. In some embodiments, the transgene encodes a chimeric antigen receptor. In some embodiments, the insert comprises a priming receptor transgene and a chimeric antigen receptor transgene.

The insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7 (2): 403-412 (2015)).

The insert can also comprise a WPRE element. WPRE elements are generally described in Higashimoto, T., et al. Gene Ther 14, 1298-1304 (2007); and Zufferey, R., et al. J Virol. 1999 April; 73 (4): 2886-92, both of which are hereby incorporated by reference.

In some embodiments, the DNA template further comprises a self-excising 2A peptide (P2A).

In some embodiments, the P2A nucleic acid is at the 3′ end of the DNA template.

In some embodiments, the DNA template further comprises a woodchuck hepatitis virus post-translational regulatory element (WPRE).

In some embodiments, the WPRE is at the 3′ end of the nucleic acid encoding the CAR and at the 5′ end of the nucleic acid encoding the priming receptor or wherein the WPRE is at the 3′ end of the nucleic acid encoding the priming receptor and at the 5′ end of the nucleic acid encoding the CAR.

### Priming Receptors

In certain aspects of the present disclosure, the priming receptor is a synthetic circuit receptor based on the Notch protein. Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synNotch priming receptor replaces the natural Notch intracellular fragment with one that causes the gene encoding the CAR to activate upon release from the priming receptor.

Notch receptors have a modular domain organization. The ectodomains of Notch receptors consist of a series of N-terminal epidermal growth factor (EGF)-like repeats that are responsible for ligand binding. In synthetic Notch receptors or priming receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of priming receptors can be used in the methods, cells, and nucleic acids as described herein. One type of priming receptor contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Tinker Notch” receptors comprise a heterologous extracellular ligand binding domain, a linking polypeptide lacking substantial sequence identity with a Notch receptor (e.g., a synthetic (GGS) n polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the priming receptor does not include a LIN-12-Notch repeat (LNR) and/or a heterodimerization domain (HD) of a Notch receptor.

### Priming Receptor Extracellular Domain

The priming receptor comprises an extracellular domain. In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′) 2 fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity. An antibody that “selectively binds” an antigen is an antigen-binding moiety that binds the antigen with high affinity and does not significantly bind other unrelated antigens.

### Transmembrane Domain

As described above, the chimeric polypeptides of the disclosure include a TMD comprising one or more ligand-inducible proteolytic cleavage sites.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, YASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the priming receptor comprises a Notch cleavage site, such as S2 or S3. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Yal-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 121), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 122), where cleavage occurs after the lysine residue. Another example of a protease cleavage site that can be included in a proteolytically cleavable linker is a thrombin cleavage site, e.g., Leu-Val-Pro-Arg (SEQ ID NO: 123). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

### Intracellular Domain

In some embodiments the priming receptor comprises one or more intracellular domains from or derived from a transcriptional regulator. Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (CAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

### DNA-Binding Domain

In some embodiments of the aspects described herein, a synthetic protein comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1a, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cashe, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu196, and TALES.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

### Juxtamembrane Domain

The ECD and the TMD, or the TMD and the ICD, can be linked to each other with a linking polypeptide, such as a juxtamembrane domain. “SynNotch” receptors comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS) n polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In some embodiments, the priming receptor comprises a juxtamembrane domain (JMD) peptide in between the extracellular domain and the transmembrane domain. In some embodiments, the priming receptor comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligimerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker linker does not comprise a protease cleavage site or a glycosylation site.

In some embodiments, the priming receptor further comprises a hinge. Hinge linkers that can be used in the priming receptor can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO: 124).

Hinge polypeptide sequences can also be derived from a CD8a hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8 a hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robo1 JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

### Stop-Transfer Sequence

In some embodiments, the priming receptor further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in priming receptors is described in WO2021061872, hereby incorporated by reference in its entirety. Non-limiting exemplary STS sequences include APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKTFD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

### Chimeric Antigen Receptors

In some embodiments, the chimeric antigen receptor includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some aspects, the chimeric antigen receptor includes an extracellular portion comprising an antigen binding domain described herein and an intracellular signaling domain. In some embodiments, an antibody or fragment includes an scFv, a VH, or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD8a or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

Chimeric antigen receptor (CAR) T cells are T cells that have been genetically engineered to produce an artificial T-cell receptor for use in immunotherapy. Chimeric antigen receptors are receptor proteins that have been engineered to confer T cells with the ability to target a specific protein. The genetic modification of lymphocytes (e.g. T cells) by incorporation of, for example, CARs, and administration of the engineered cells to a subject is an example of “adoptive cell therapy”. As used herein, the term “adoptive cell therapy” refers to cell-based immunotherapy for transfusion of autologous or allogeneic lymphocytes, referred to as T cells or B cells. In this CAR therapy approach, cells are expanded and cultured ex vivo and genetically modified, prior to transfusion.

The expression of CARs allows the engineered T-cells to target and bind specific proteins, for example, tumor antigens. In CAR therapy, T-cells are harvested from a subject-they can be autologous T-cells from the subject own blood or from a donor that will not be receiving the CAR therapy. Once isolated, the T-cells are genetically modified with a CAR, expanded ex vivo, and administered to the subject (i.e. patient) by, e.g. infusion.

The CARs may be introduced into the T-cells using, for example, a site-specific technique. With site specific integration of the transgenes (e.g. CARs), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered CAR T cells have applications to immune-oncology. The CAR, for example, can be selected to target a specific tumor antigen. Examples of cancers that can be effectively targeted using CAR T cells are blood cancers. In some embodiments, CAR T cell therapy can be used to treat solid tumors.

### Chimeric Antigen Receptor Extracellular Domain

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. In some embodiments, the antigen-binding moiety is selected from the group consisting of an antibody, a nanobody, a diabody, a triabody, or a minibody, a F(ab′) 2 fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, and/or CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the CAR comprises a CD8a or CD28 TMD.

### CAR Hinge

In some embodiments, the CAR further includes a spacer, which may be or include at least a portion of an immunoglobulin constant region or variant or modified version thereof, such as a hinge region, e.g., a CD8a hinge, an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8a hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8a CD8a, truncated CD8a, or CD28 hinge domain.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta. In some embodiments, the intracellular activation domain comprises a CD33 domain.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16.

In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD8a transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g. containing only the VH region), VH domains, and scFvs, described herein, a spacer such as a CD8a hinge, a CD8a transmembrane domain, a 4-1BB intracellular signaling domain, and a CD3 zeta signaling domain. In some embodiments, the CAR includes an antibody or fragment, including sdAbs and scFvs described herein, a spacer such as a CD8a hinge, a CD8a transmembrane domain, a 4-1BB intracellular signaling domain, and a CD3 zeta signaling domain.

Transgenes expressing the priming receptor and CAR system may be introduced into cells, such as a T cell, using, for example, a site-specific technique. With site specific integration of the transgenes (e.g. priming receptor and CAR), the transgenes may be targeted to a safe harbor locus or TRAC. Examples of site-specific techniques for integration into the safe harbor loci include, without limitation, homology-dependent engineering using nucleases and homology independent targeted insertion using Cas9.

The engineered cells have applications to immune-oncology. The priming receptor and CAR, for example, can be selected to target different specific tumor antigens. Examples of cancers that can be effectively targeted using such cells are blood cancers or solid cancers. In some embodiments, immune cell therapy can be used to treat solid tumors.

In some embodiments, upon ligation of the CAR, the cytoplasmic domain or intracellular signaling domain of the receptor activates at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the receptor. In other embodiments, the receptor does not include a component for generating a costimulatory signal. In some aspects, an additional receptor is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or modified synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

In some cases, CARs are referred to as first, second, and/or third generation CARs. In some aspects, a first generation CAR is one that solely provides a CD3-chain induced signal upon antigen binding; in some aspects, a second-generation CAR is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing an antibody or fragment described herein. In some aspects, the chimeric antigen receptor includes an extracellular portion containing an antibody or fragment described herein and an intracellular signaling domain. In some embodiments, an antibody or fragment includes an scFv or a single-domain VH antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer. In some embodiments, the transmembrane domain of the receptor, e.g., the CAR, is a

transmembrane domain of human CD28 or variant thereof, e.g., a 27-amino acid transmembrane domain of a human CD28 (Accession No.: P10747.1).

In some embodiments, the chimeric antigen receptor contains an intracellular domain of a T cell costimulatory molecule. In some aspects, the T cell costimulatory molecule is CD28 or 4-1BB.

In some embodiments, the intracellular signaling domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular domain comprises an intracellular costimulatory signaling domain of 41BB or functional variant or portion thereof, such as a 42-amino acid cytoplasmic domain of a human 4-1BB (Accession No. Q07011.1) or functional variant or portion thereof.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

### Engineered Cells

Also provided herein are engineered cells comprising at least one DNA template non-virally inserted into a target region of the genome of the cell. In some embodiments, the size of the DNA template is greater than or equal to about 0.3 kb, 1 kb, 2 kb, 3 kb, 4 kb, 4.5 or 5 kilobase pairs (kb).

In some embodiments, the size of the DNA template is greater than or equal to about 0.3 kb, 0.5 kb, 1.0 kb, 1.5 kb, 2.0 kb, 2.5 kb, 3.0 kb, 3.5 kb, 4.0 kb, 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb. 9.9 kb, 10.0 kb, 10.1 kb, 10.2 kb, 10.3 kb, 10.4 kb, 10.5 kb, 10.6 kb, 10.7 kb, 10.8 kb, 10.9 kb, 11.0 kb, 11.1 kb, 11.2 kb, 11.3 kb, 11.4 kb, 11.5 kb, 11.6 kb, 11.7 kb, 11.8 kb, 11.9 kb, 12.0 kb, 12.1 kb, 12.2 kb, 12.3 kb, 12.4 kb, 12.5 kb, 12.6 kb, 12.7 kb, 12.8 kb, 12.9 kb, 13.0 kb, or larger, or any size DNA template in between these sizes.

In some embodiments, the size of the DNA template is about 0.3 kb to about 13 kb, about 0.3 kb to about 0.5 kb, about 0.3 kb to about 1 kb, about 0.3 kb to about 4 kb, about 0.3 kb to about 3 kb, about 0.3 kb to about 5 kb, about 0.3 kb to about 7 kb, about 0.3 kb to about 10 kb, about 0.5 kb to about 1 kb, about 0.5 kb to about 3 kb, about 0.5 kb to about 5 kb, about 0.5 kb to about 7 kb, about 0.5 kb to about 10 kb, about 0.5 kb to about 13 kb, about 1 kb to about 3 kb, about 1 kb to about 5 kb, about 1 kb to about 7 kb, about 1 kb to about 10 kb, about 1 kb to about 13 kb, about 5 kb to about 13 kb, 5 kb to about 13 kb, about 5 kb to about 9 kb, about 5 kb to about 8 kb, about 5 kb to about 7 kb, about 5 kb to about 6 kb, about kb 6 to about 13 kb, about kb, 6 to about 10 kb, about 6 kb to about 9 kb, about 6 kb to about 8 kb, about 6 kb to about 7 kb, about 7 kb to about 13 kb, about 7 kb to about 10 kb, about 7 kb to about 9 kb, about 7 kb to about 8 kb, about 8 kb to about 13 kb, about 8 kb to about 10 kb, about 8 kb to about 9 kb, about 9 kb to about 13 kb, about 9 kb to about 10 kb, about 10 kb to about 13 kb, or about 11 kb to about 13 kb.

In some embodiments, the DNA template comprises a transgene encoding a protein. The protein can be any protein of interest, such as, but not limited to a CAR, a priming receptor, a TCR, an antibody.

A cell comprising an insert at a target locus or safe harbor site as described in the present disclosure can be referred to as an engineered cell. In some embodiments, the immune cell is any cell that can give rise to a pluripotent immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor cell. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an adaptive immune cell, an innate immune cell, a T cell or a T cell progenitor. Non-limiting examples of immune cells that are contemplated in the present disclosure include T cell, B cell, natural killer (NK) cell, NKT/iNKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

Also provided herein are populations of cells comprising a plurality of the primary immune cell. In some embodiments, the genome of at least 20%, 30%, 40%, 50%, 60%, 70%, 80%, 90%, 95%, 99% or greater of the cells comprises a targeted insertion of a heterologous DNA template, wherein the DNA template is at least about 5 kb in size.

### Therapeutic Applications

For therapeutic applications, the engineered cells, populations thereof, or compositions thereof are administered to a subject, generally a mammal, generally a human, in an effective amount.

The engineered cells may be administered to a subject by infusion (e.g., continuous infusion over a period of time) or other modes of administration known to those of ordinary skill in the art.

The engineered cells provided herein can be administered as part of a pharmaceutical compositions. In some embodiments, the present disclosure provides compositions comprising a guide RNA of the present disclosure. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. In some embodiments, the engineered cells are immune cells. In some embodiments, the engineered cells are lymphocytes. In some embodiments, the engineered cells are T cells or T cell progenitors.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

Various diseases treated using the engineered cells, populations thereof, or compositions thereof are provided herein. Non-limiting examples of such diseases include alopecia areata, autoimmune hemolytic anemia, autoimmune hepatitis, cancer, dermatomyositis, diabetes (type 1), certain juvenile idiopathic arthritis, glomerulonephritis, Graves' disease, Guillain Valley Syndrome, idiopathic thrombocytopenia purpura, myasthenia gravis, certain myocarditis, multiple sclerosis, pemphigus/pemphigoid, pernicious anemia, polyarteritis nodosa, polymyositis, primary bile With cirrhosis, psoriasis, rheumatoid arthritis, scleroderma/systemic sclerosis, Sjogren's syndrome, systemic lupus erythematosus, certain thyroiditis, certain uveitis, vitiligo, multiple vasculitis (Wegener)); autoimmune disorders including, but not limited to, granulomatosis; hematopoietic tumors including but not limited to acute and chronic leukemia, lymphoma, multiple myeloma and myelodysplastic syndrome; tumors of the prostate, breast, lung, colon, uterus, skin, liver, bone, pancreas, ovary, testis, bladder, kidney, head, neck, stomach, cervix, rectum, larynx, or esophagus solid tumors; HIV (human immunodeficiency virus) related disorders, RSV (respiratory syncytial virus) related disorders; EBV (Epstein-Barr virus) related disorders; CMV (cytomegalovirus) related disorders; and infectious diseases including, but not limited to, adenovirus-related disorders and BK polyomavirus-related disorders.

Cancers that can be treated with the engineered cells (e.g., CAR T-cells) of the present disclosure, populations thereof, or compositions thereof include blood cancers. In some embodiments, the cancer treated using the engineered cells (e.g., CAR T-cells) described herein, populations thereof, or compositions thereof is a hematologic malignancy or leukemia. In some embodiments, the engineered cells (e.g., CAR T-cells) described herein, populations thereof, or compositions thereof are used for the treatment of acute lymphoblastic leukemia (ALL) or diffuse large B-cell lymphoma (DLBCL). In some embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia. Examples of cancers treatable using the engineered cells (e.g., CAR T-cells) described herein include, without limitation, breast cancer, ovarian cancer, esophageal cancer, bladder or gastric cancer, salivary duct carcinoma, salivary duct carcinomas, adenocarcinoma of the lung or aggressive forms of uterine cancer, such as uterine serous endometrial carcinoma. In some other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some embodiments, the present disclosure provides methods of treating a subject in need of treatment by administering to the subject a composition comprising any of the engineered cells described herein. As used, the terms “treat,” “treatment,” and the like refer generally to obtaining a desired pharmacological and/or physiological effect. That effect is preventive in terms of complete or partial prevention of the disease and/or therapeutic in terms of partial or complete cure of the disease and/or adverse effects resulting from the disease. The term “treatment”, as used herein, encompasses any treatment of a disease in a subject (e.g., mammal, e.g., human). Treatment may also refer to the administration of the engineered cells provided herein to a subject that is susceptible to the disease but has not yet been diagnosed as suffering from it, including preventing the disease from occurring; inhibiting disease progression; or reducing the disease (i.e., causing a regression of the disease). Further, treatment may stabilize or reduce undesirable clinical symptoms in subjects (e.g., patients). The cells provided herein populations thereof, or compositions thereof may be administered before, during or after the occurrence of the disease or injury.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition. In certain embodiments, a subject in need of cell therapy includes, but is not limited to, a bone marrow transplant or stem cell transplant candidate, a subject who has received chemotherapy or radiation therapy, a hyperproliferative disease or cancer (e.g., a hematopoietic system), a subject having or at risk of developing a hyperproliferative disease or cancer), a subject having or at risk of developing a tumor (e.g., solid tumor), viral infection or virus. It is also intended to encompass subjects suffering from or at risk of suffering from a disease associated with an infection.

In some embodiments, the present disclosure provides a composition of the present disclosure along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

### Combination Therapies

In some embodiments, an engineered cells of the present disclosure or composition thereof is administered with at least one additional therapeutic agent. Any suitable additional therapeutic agent may be administered with an engineered cell provided herein, populations thereof, or compositions thereof. In some aspects, the additional therapeutic agent is selected from radiation, an ophthalmologic agent, a cytotoxic agent, a chemotherapeutic agent, a cytostatic agent, an anti-hormonal agent, an immunostimulatory agent, an anti-angiogenic agent, and combinations thereof.

In some embodiments, an engineered cell of the present disclosure or composition thereof is administered with a steroid. The administration of a steroid can prevent or mitigate the risk of a subject receiving the engineered cell(s) or composition thereof having an autoimmune reaction.

The additional therapeutic agent may be administered by any suitable means. In some embodiments, the engineered cells described herein, populations thereof, or compositions thereof and the additional therapeutic agent is administered in the same pharmaceutical composition, e.g. by infusion. In some embodiments, the engineered cells described herein and additional therapeutic agent are included in different pharmaceutical compositions.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration. Generally, modes of administration include, without limitation, intravitreal, subretinal, suprachoroidal, intraarterial, intradermal, intramuscular, intraperitoneal, intravenous, nasal, parenteral, topical, pulmonary, and subcutaneous routes.

In embodiments where the engineered cells provided herein and the additional therapeutic agent are included in different pharmaceutical compositions, administration of the engineered cells provided herein can occur prior to, simultaneously, and/or following, administration of the additional therapeutic agent.

### Pharmaceutical Compositions of the Invention

Methods for treatment of cancer diseases are also encompassed by the present disclosure. Said methods include administering a therapeutically effective amount of an engineered cell as described herein. The engineered cells can be formulated in pharmaceutical compositions. These compositions can comprise, in addition to one or more of the engineered cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Increased Editing Efficiency with RNase Inhibitor

### Materials and Methods

Freshly isolated or frozen and thawed isolated T cells were activated with CD3/CD28 Dynabeads (Thermo Fisher Scientific, 43500D or 40203D) at either a 3:1 or 2:1 bead:cell ratio for 2 days in complete media (TexMACS (Miltenyi Biotec, 170-076-306)+3% PLTGold (Mill Creek Life Sciences, PLTGold100GMP)+12.5 ng/ml IL-7+12.5 ng/ml IL-15) in gas permeable bags (Charter Medical, EXP-3L) at 37° C., 5% CO2. Beads were then removed and cells were concentrated and buffer exchanged into Genome Editing Buffer (Thermo Fisher Scientific, A4998002 or A4998001). For conditions with RNase inhibitor, RNase inhibitor (New England Biolabs, M0314L or G8005B-1ML) was added to cells and allowed to incubate for at least 10 minutes prior to payload addition. Payload (plasmid DNA and ribonucleoprotein (RNP) comprised of Cas9 protein complexed with sgRNA targeting GS94) was then added to cells and cells were electroporated using the CTS Xenon Electroporation System (Thermo Fisher Scientific) using either the Xenon SingleShot (Thermo Fisher Scientific, A50305) or MultiShot consumable (Thermo Fisher Scientific, A53445). Post-electroporation, cells were transferred to GRex vessels (Wilson Wolf Manufacturing, 80660M or 81100-CS) containing complete media at 1/10th the fill volume of the vessel. One day post-electroporation, complete media was added to full volume. Cells were then sampled for cell counts and flow cytometry to measure percent knock-in at 4 to 7 days post-electroporation.

### Results

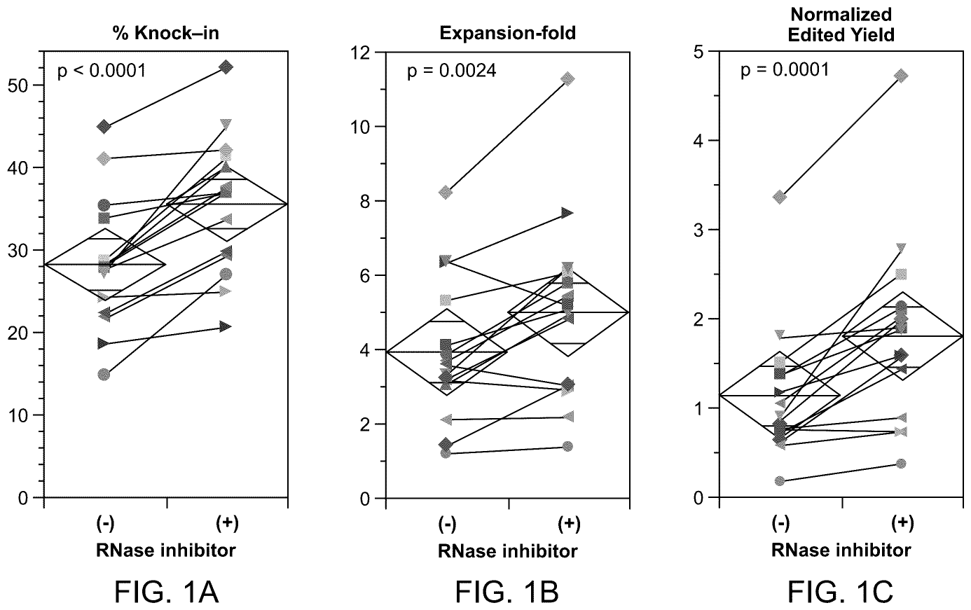

The use of RNase inhibitor increased the percent knock in, cell expansion, and normalized edited yield of T cells as compared to no RNase inhibitor (FIG. 1A-C). FIG. 1 shows a summary of the % knock in (editing, FIG. 1A), cell expansion (FIG. 1B), and edited T cell yield (FIG. 1C) for cells electroporated to insert an exemplary transgene without (−) and with (+) RNase inhibitor using the Xenon platform for 15 unique donors. P values from one-sided, matched t-test, 15 unique donors. Inclusion of an RNase inhibitor increased each value assessed as compared to cells electroporated without an RNase inhibitor. The addition of RNase inhibitor consistently increased cell editing efficiency (percent knock-in) in 15 of 15 donors tested using an exemplary priming receptor as a transgene insertion (FIG. 1A) and the Xenon SingleShot platform. Cell editing efficiency was improved by approximately 26% on average, but in some cases reached almost as high as 1-fold improvement. In addition, cell growth (expansion-fold) was also generally improved with the addition of RNase inhibitor (FIG. 1B), resulting in similar or higher edited T cell yields in 15 of 15 donors (FIG. 1C).

FIGS. 2A and 2B, show that inclusion of RNase inhibitor (right bars) prior to electroporation also improved KI and edited yields in the Xenon MultiShot in both donors tested as compared to cells incubated without an RNase inhibitor (left bars). FIG. 2A shows the percent of cells with the knock-in of the exemplary transgene after incubation with or without an RNase inhibitor. FIG. 2B shows a normalized edited T cell yield (the total number of edited cells divided by the number of cells electroporated) after incubation with or without an RNase inhibitor. Across the 2 donors tested, a 30-50% increase in editing efficiency and 20-110% increase in edited T cell yields was observed with the addition of an RNase inhibitor.

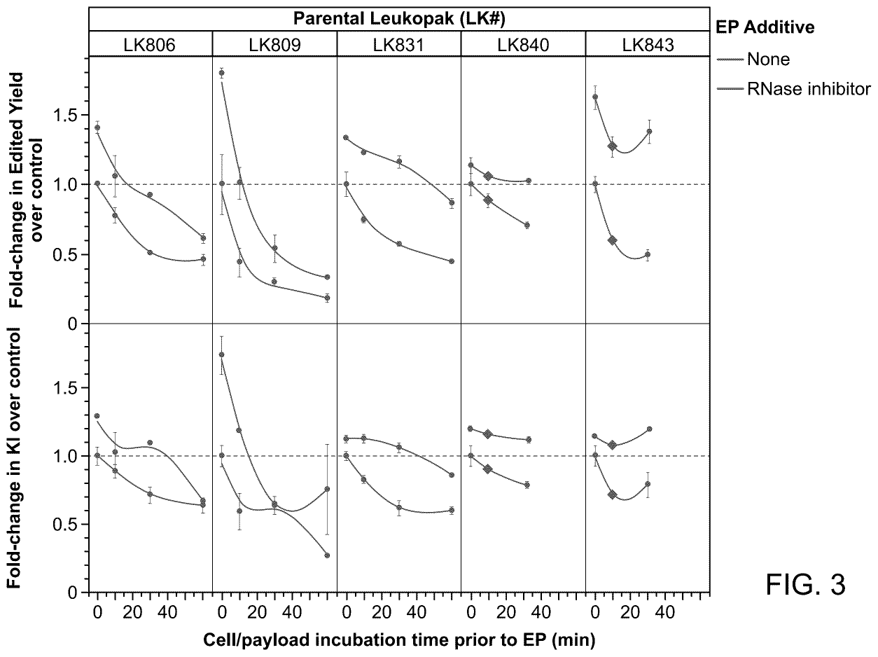

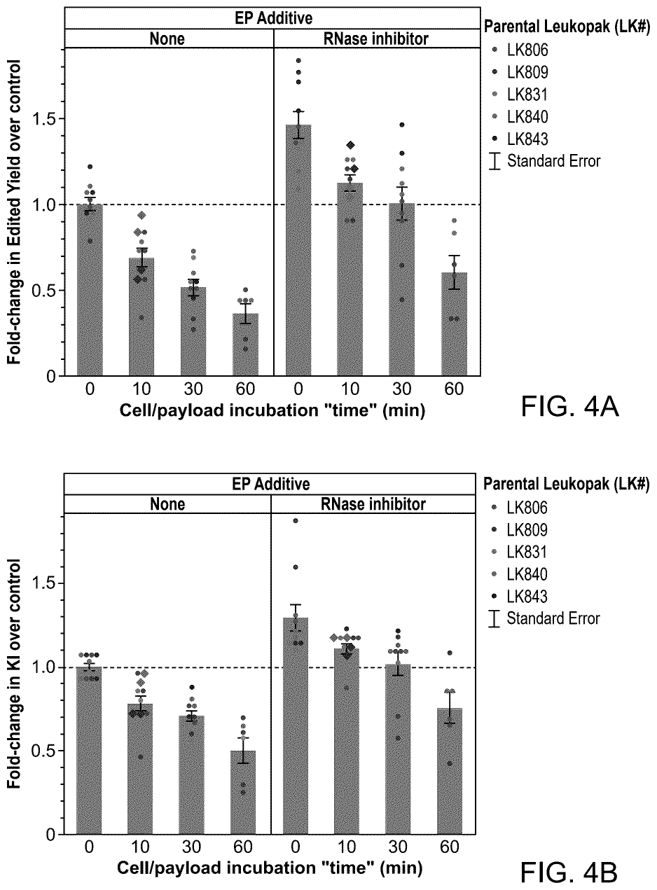

Editing efficiency and edited T cell yields can be negatively impacted by longer cell and payload exposure times prior to electroporation. However, the addition of RNase inhibitor consistently increased edited T cell yields in 5 of 5 donors tested using an exemplary priming receptor as a transgene insertion. In cells with no RNase inhibitor, a 0 to 60 minute cell/payload incubation time prior to electroporation resulted in decreases in both edited T cell yields and editing efficiencies in 5 of 5 donors (FIG. 3). A 30 minute cell/payload incubation time prior to electroporation with no RNase inhibitor resulted in an average of a 30% decrease in editing efficiency and 50% decrease in edited T cell yields (FIG. 4A and FIG. 4B). In contrast, addition of RNase inhibitor prior to electroporation improved both editing efficiencies and edited T cell yields across all timepoints (FIG. 3, FIGS. 4A and 4B). Similar edited T cell yields were observed with the 0 min incubation time without RNase inhibitor and the 10 min incubation time with RNase inhibitor (FIG. 3 and FIG. 4B). In addition, at the 0 min incubation, addition of RNase inhibitor resulted in a 1.5-fold increase in edited T cell yields (FIG. 4B and FIG. 3).

Thus, RNase inhibitor consistently increased % KI and edited yields in all donor cells tested. Addition of an RNase inhibitor improved edited yields across all time points (FIGS. 4A and 4B).

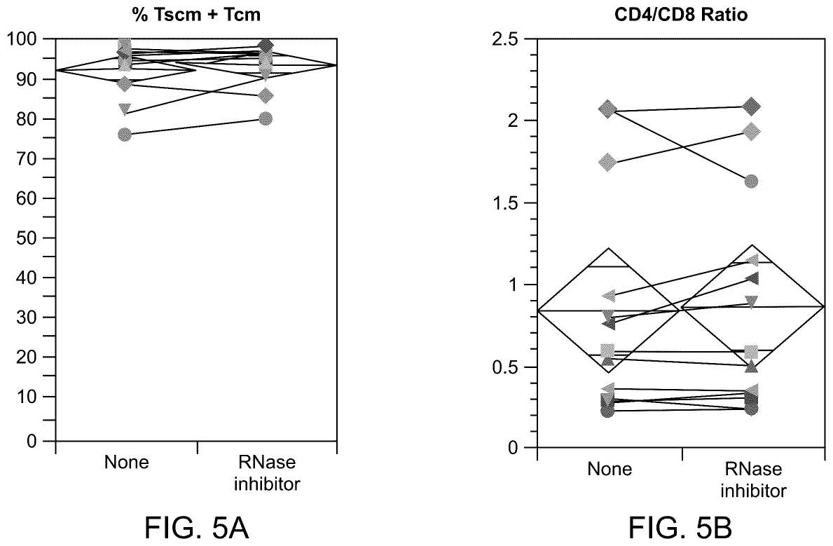

The addition of the RNase inhibitor did not adversely affect the cell phenotype (FIGS. 5A and 5B). In six experiments using thirteen different donors, the addition of RNase inhibitor did not alter the mean proportion of “younger memory” T cells (stem cell memory T cells/CD45RA+CCR7+ and central memory T cells/CD45RA−CCR7+) (FIG. 5A) or ratio of CD4 to CD8 T cells (FIG. 5B). p-values are from a two-sided matched pairs t test.

Thus, without wishing to be bound by theory, addition of an RNase inhibitor improved the gene editing performance relative to all conditions without an RNase inhibitor.

### Example 2: Increased Editing Efficiency with N acetyl-L-cysteine (NAC)

### Materials and Methods

### Cell Activation

CD4/CD8 isolated human primary T cells were removed from liquid nitrogen and thawed in 1 ml of warm media (TexMACS+10% human AB serum) for each 1 ml frozen vial.

All the cells from the same donor were pooled and transferred to a 50 ml tube containing the warm media, 10 ml per frozen vial. The cells were centrifuged at 400 g for 5 minutes. After centrifugation, 400e6 cells were cultured in a total volume of 200 ml hTCM (TexMACs+3% Human AB Serum+12.5 ng/ml IL-7+12.5 ng/ml IL-15) in Grex 100M vessel with CTS Dynabeads CD3/CD28 in 3:1 beads to cells ratio. After 2 days, the cells were debeaded and subjected to electroporation

### Lonza Electroporation

T cells were thawed and activated as described above. RNPs were complexed at room temperature for 10 minutes by mixing a target sgRNA and Cas9 in a 5:1 ratio. Lonza P3 buffer was added to each RNP complex to make an electroporation (EP) reaction mixture. 1 mg/ml DNA (GFP-expressing HR repair template) was diluted in the Lonza P3 buffer.

To prepare neutralized NAC, NAC in powdered form (Sigma, A7250-5G) was dissolved in 50 ml of PBS. NaOH was added to neutralize NAC in 1:1 molar ratio by adding 1 ml of 10M NaOH for every 16.3 ml of NAC to a final 578.035 mM stock concentration. The solution was sterile filtered. For electroporation, the stock concentration of neutralized NAC was diluted in the P3 buffer to 50 mM. A 96 well Lumox plate was used for recovery. 250 ul of hTCM with and without 5 mM NAC was added to each well and incubated at 37° C. After the cells were debeaded, 1e6 cells per reaction were centrifuged at 90 g for 10 min and the supernatant removed. For NAC conditions, cell pellets were resuspended in P3 buffer, neutralized NAC, RNP and DNA. The reaction concentration of the NAC was 5 mM. For control conditions without NAC, the cell pellets were resuspended in P3 buffer, RNP and DNA. The cells were electroporated using the Lonza 384HT with the EH115 pulse code.

After electroporation, cells were recovered first in the electroporation plate for 15 min at room temperature and then recovered in hTCM with and without 5 mM NAC in a 96 well lumox plate. The cells were split at day 1 post-electroporation and day 3 post-electroporation and new media with and without 5 mM NAC added. At day 5 post-electroporation, a portion of cells was collected for a Knock In (KI) efficiency analysis via flow cytometry. Approximately 2e5 cells were collected per sample for flow analysis. The cells were resuspended in BSA staining buffer, CountBright counting beads and TO-PRO-3 and stained for 30 minutes at 4C. The cells were washed twice and resuspended wash buffer volume for analysis. The samples were analyzed on the Attune for GFP (indicating successful knock-in) and TO-PRO-3 (indicating nonviability).

### Xenon Large Scale Electroporation

T cells were thawed and activated as described above. After the cells were activated for 2 days, cells were debeaded prior to electroporation. 25e6 cells were used per reaction. RNPs were complexed at room temperature for 10 minutes by mixing a target sgRNA and Cas9 in a 4:1 ratio. Neutralized NAC stock was diluted in Xenon GE buffer to a final concentration of 50 mM. A Grex6M plate was prepared by adding hTCM to each well and warmed at 37° C. Cells were centrifuged, PBS was added to the cell pellet, and the cells were centrifuged at 400 g for 5 min. For the NAC conditions, the cell pellet was resuspended in Thermo GE buffer with RNP and DNA (GFP-expressing HR repair template) and 5 mM NAC. For the no NAC control conditions, the cell pellet was resuspended in Thermo GE buffer with RNP and DNA. For each replicate, the cells/payload mixture was transferred to a Xenon Single Shot cartridge and the cells were electroporated in a Thermo Xenon unit at 2300V, 4 pulses and 3 ms. After electroporation, the cells were recovered in the cartridge for 10 min at room temperature. The cells were transferred to the prepared plate with hTCM with or without 5 mM of NAC (final concentration). One day post-EP, hTCM with and without 5 mM NAC was added to each of the appropriate wells. At day 3 post-electroporation, fresh hTCM with and without 5 mM NAC was added to the cells. At day 5 post-electroporation, cells were stained and analyzed for KI efficiency on the Attune using the flow cytometry staining as described above.

### Comparison of NAC Concentrations

The cell editing efficiency and yield using 2.5 mM NAC and 5 mM NAC were also compared. Cells were electroporated using the Xenon protocol described above, with either 2.5 mM NAC or 5 mM NAC supplement in the electroporation buffer and 5 days post electroporation.

### Results

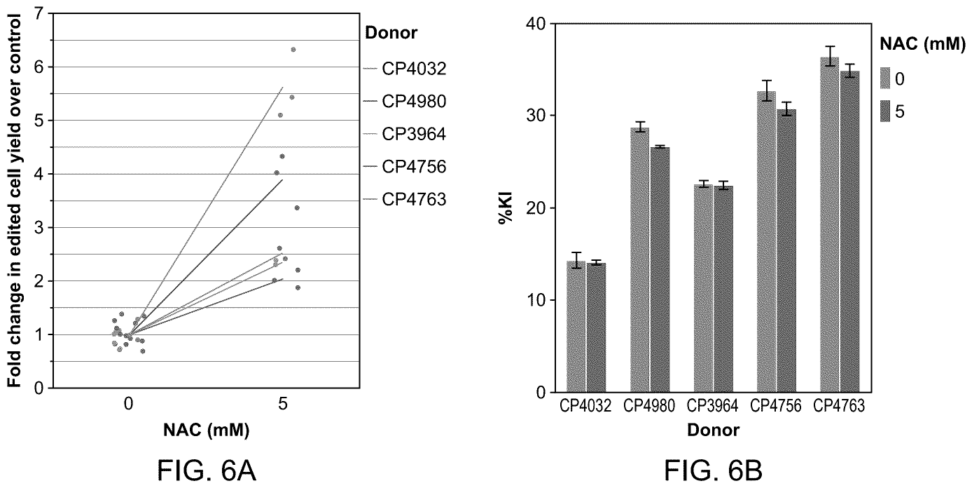

FIG. 6A provides the fold change in total edited cells yield in the NAC treated samples as normalized to the no drug control after the Lonza electroporation assay. Cells from five donors treated with 5 mM neutralized NAC during electroporation demonstrated improved total edited cell yield (2 to 6 fold improvement) in Lonza small scale electroporation (FIG. 6A). In addition, no or minimal change in KI efficiency was observed in the NAC treated samples as compared to the no NAC control (FIG. 6B). Thus, addition of NAC during electroporation increased the edited cell yield and did not adversely impact the knock in efficiency of the transgene.

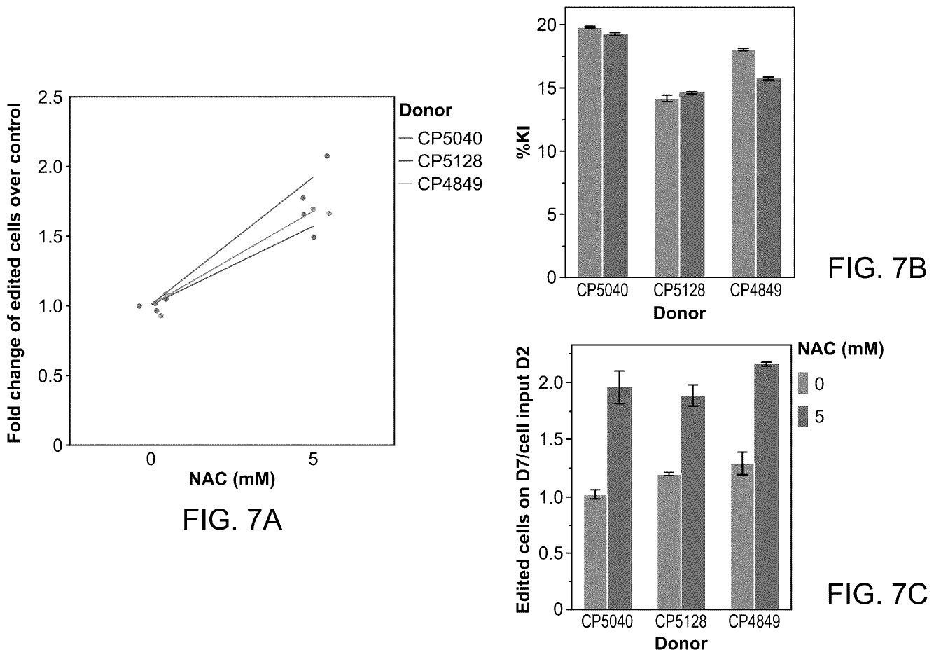

FIG. 7A provides the fold change in total edited cells yield in the NAC treated samples as normalized to the no drug control after the Xenon electroporation assay shows.

Inclusion of 5 mM NAC during electroporation resulted in a 2 fold increase in edited cell yield in 3 donors. FIG. 7C shows the normalized edited cell count on D7 as normalized to cell input on D2. FIG. 7B shows the % KI of the transgene in cells incubated with NAC during electroporation. No or minimal change in KI efficiency was observed in the NAC treated samples as compared to the no NAC control (FIG. 7B). Thus, addition of NAC during electroporation increased the edited cell yield and did not adversely impact the knock in efficiency of the transgene.

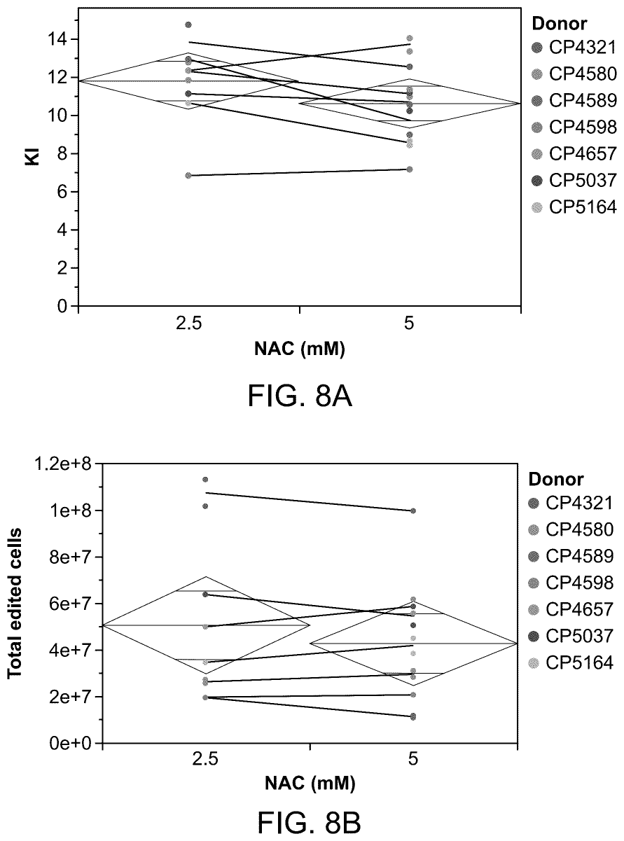

FIG. 8A shows the % KI of the target gene after electroporation with 2.5 mM or 5 mM NAC. FIG. 8B shows the total number of edited cells after electroporation with 2.5 mM NAC or 5 mM NAC. No significant difference was observed in the total edited cell yield of the 2.5 mM NAC as compared to the 5 mM neutralized NAC buffer in the 7 donor cells. However, the cells electroporated in the presence of 2.5 mM neutralized NAC showed higher KI efficiency in cells from 5 out of 7 donors as compared to the 5 mM NAC samples.

An additional assay comparing 5 mM and 2.5 mM NAC in both Cellvive and PLTGold media also showed better editing outcomes in 2.5 mM NAC conditions (data not shown).

Without wishing to be bound by theory, application of an electric field during the electroporation process to create cell membrane permeability can induce production of reactive oxygen species (ROS) and cause injury to the plasmid membrane. If the cells are unable to repair the cell membrane, cell death can occur following an increase in ROS levels, ATP depletion, and calcium influx. Electroporation itself also produces ROS due to charge transfer reactions at the electrodes. Thus, without wishing to be bound by theory, inclusion of an ROS scavenger such as N acetyl-L-cysteine (NAC) may increase cell viability by protecting cells during electroporation from ROS-mediated cell death resulting from cell membrane damage.

### Example 3: Increased Editing Efficiency with Sorbitol

### Materials and Methods

Cells were removed from liquid nitrogen and thawed two days prior to electroporation. Thaw media (TexMACS+10% human AB serum) was added to the cells before donor cells were pooled and centrifuged at 400 g for 4 minutes to form a pellet. The cells were then added to GRex100M in hTCM (TexMACS+3% Human AB Serum+12.5 ng/ml IL-7+12.5 ng/ml IL-15) with 15 ug/mL gentamicin and 3 times the cell number of CTS Dynabeads CD3/CD28.

After two days the cells were debeaded and washed with PBS. RNPs were complexed at room temperature for 10 minutes by mixing a target sgRNA and Cas9 at a 4:1 ratio. The cell pellet was then resuspended in either standard Xenon Electroporation (EP) buffer or Xenon Electroporation buffer with 200 mM sterile filtered sorbitol. Plasmid with a transgene (GFP-expressing HDR template) was then added to the cells in electroporation buffer with the RNP. The mixture was then electroporated in a Xenon electroporator, with settings 2300V, 4 pulses, 3 ms, and 0.5 s between pulses. The cells were added to fresh hTCM in a GRex 6 Well M plate and returned to the incubator. One day after the electroporation fresh hTCM was added to each sample. Three days after the electroporation, the samples were split and resuspended in fresh hTCM. Cells were harvested for flow cytometry on day 5 with CountBright counting beads and TO-PRO-3. Samples were run on an Attune flow cytometer for GFP and TO-PRO-3 viability staining.

### Results

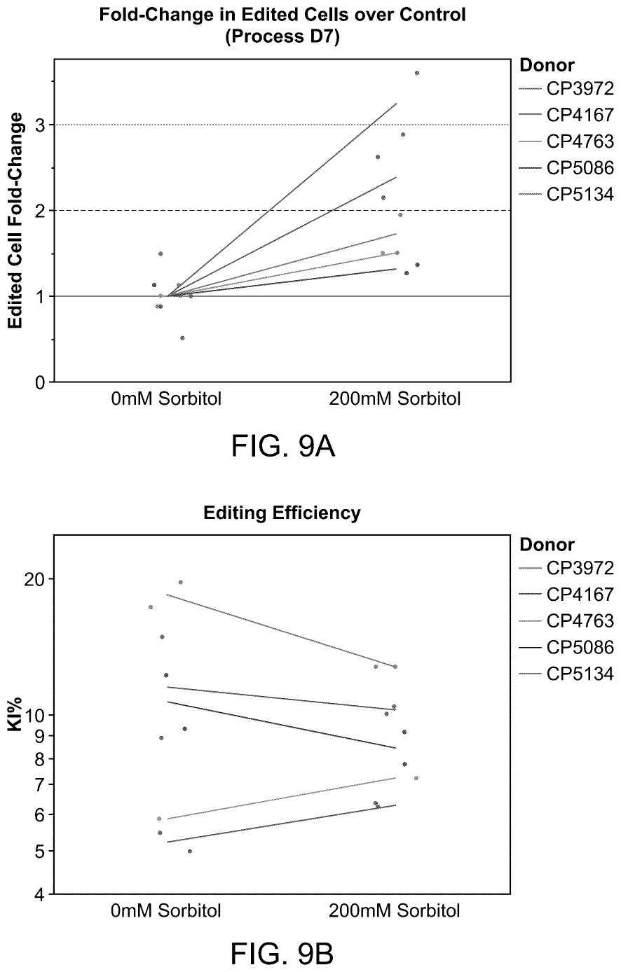

FIG. 9A shows the fold change in total edited cells normalized to the starting number of cells in the electroporation in the presence of 200 mM sorbitol or in the absence of sorbitol (0 mM). FIG. 9B shows the % KI editing efficiency in the presence of 200 mM sorbitol or in the absence of sorbitol (0 mM). The addition of 200 mM sorbitol to the Xenon electroporation buffer resulted in a 2.02±0.79-fold increase in edited cell yield across cells from five donors. 200 mM sorbitol in Xenon electroporation buffer improved edited cell yield in every donor tested, with increased editing effect of 27% to 224% (FIG. 9A). There was minimal effect on the knock in percentage with sorbitol (FIG. 9B). Overall, the addition of sorbitol to electroporation buffer improved the yield of viable cells after electroporation while having a minimal difference in editing efficiency (KI %) resulting in an overall increase in total edited cells.

Without wishing to be bound by theory, during electroporation, pores are introduced into the cell membrane that allow external ions to enter the cell. Such pores can also expand further into tears that cannot be repaired. Both the external ions and the tears can lead to cell bursting and cell death. Thus, tuning the osmolarity by using buffer additives such as sorbitol or glycerol may increase the pressure outside of the membrane to stabilize these pores with a compressive force and reduce the outward osmotic pressure to prevent the cells from bursting.

### Example 4: Increased Editing Efficiency with Exogenous DNA Repair Modulation and/or Homologous Recombination Proteins

### Materials and Methods

Successful integration of a transgene relies primarily on homologous dependent repair (HDR), a high fidelity DNA repair mechanism. HDR is in kinetic competition with NHEJ, a lower fidelity process that is not known to enable insertion of large trans genes. Existing methods to boost KI efficiency include the use of small molecule inhibitors of NHEJ, modifications to the DNA molecule bearing the insert cassette, and modifications of the CRISPR-Cas protein that either enhance HDR or inhibit NHEJ. However, none of these methods have proven effective when trying to insert very large (>8 kb) expression cassettes in activated, primary human T cells.

In order to induce HDR over error-prone DNA repair mechanisms such as non-homologous end joining (NHEJ), a plasmid library consisting of genes involved in HDR DNA repair, antagonists of NHEJ, and cell-cycle modulation was designed. Genes were screened via electroporation for efficacy in improving gene editing performance, resulting in identification of three genes that improved knock in (KI) of an exemplary transgene(s) (e.g., a heterologous protein or proteins, such as a priming receptor and/or a CAR) in T cells when expressed from a separate, non-integrating, non-replicating plasmid (episomal plasmid). 84 proteins were tested for increased KI efficacy. Luciferase was used as a control. Proteins were tested with and without a nuclear localization signal (NLS) tag. Proteins were transiently expressed from plasmids with the same common backbone sequence. The plasmid included a CMV enhancer, a CAG promoter, a hybrid intron, the gene of interest, and a bGH polyA. The plasmid was delivered alongside the insertion cassette comprising the DNA template and the Cas9 RNP. The plasmid was non-integrating and non-replicative.

Modulation of editing efficiency usually relies on small molecule inhibitors, which can have unintended off-target effects. This design emphasizes transient expression that peaks within 24 hours, capturing the HDR time-window.

To test the plasmids at a 96-well scale, each well received 1×10⁶ activated T cells in electroporation buffer, a Cas9 RNP targeting a safe-harbor locus, and a plasmid encoding an exemplary CAR and a GFP marker, flanked by 5′ and 3′ homology sequences for insertion. The insertion cassette was 9 kb in length. Plasmids encoding potential HDR enhancing genes were added to individual wells in an arrayed format. Cells were electroplated using a system developed in-house. Cells were transferred to human T cell media and expanded for 5 days. Flow cytometry was used to calculate editing efficiency of each well.

Plasmids that enhanced editing efficiency were used in larger scale testing.

For follow up testing at larger scale using the Xenon electroporator, 50×10⁶ activated T cells were electroporated using the manufacturer's recommended protocol. Cells received Cas9 RNP targeting a safe-harbor locus, and 20 μg of a plasmid encoding an exemplary CAR and a GFP marker, flanked by 5′ and 3′ homology sequences for insertion. The insertion cassette was 9 kb in length. Each independent sample also received 20 μg of one plasmid encoding the HDR-enhancing genes selected from the 96-well scale experiment. Cells were transferred to human T cell media and expanded for 5 days. Flow cytometry was used to calculate editing efficiency of each well.

### Results

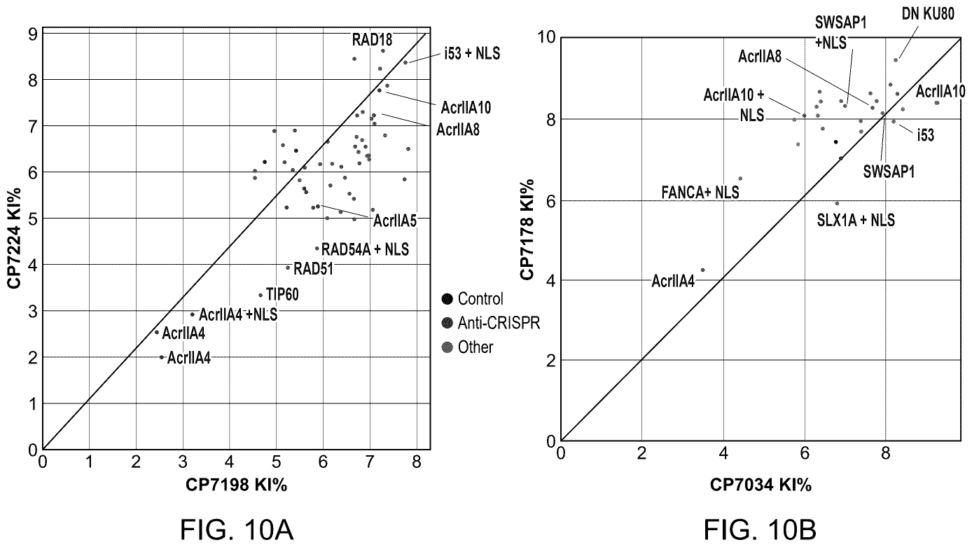

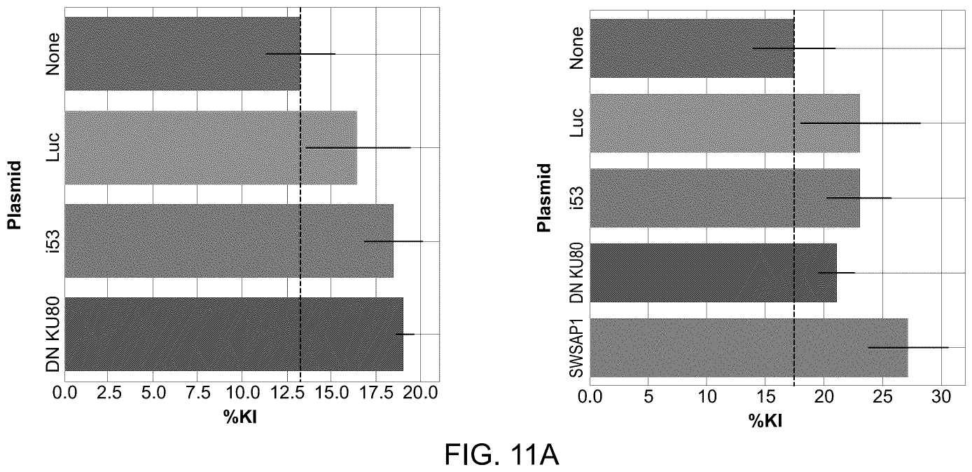

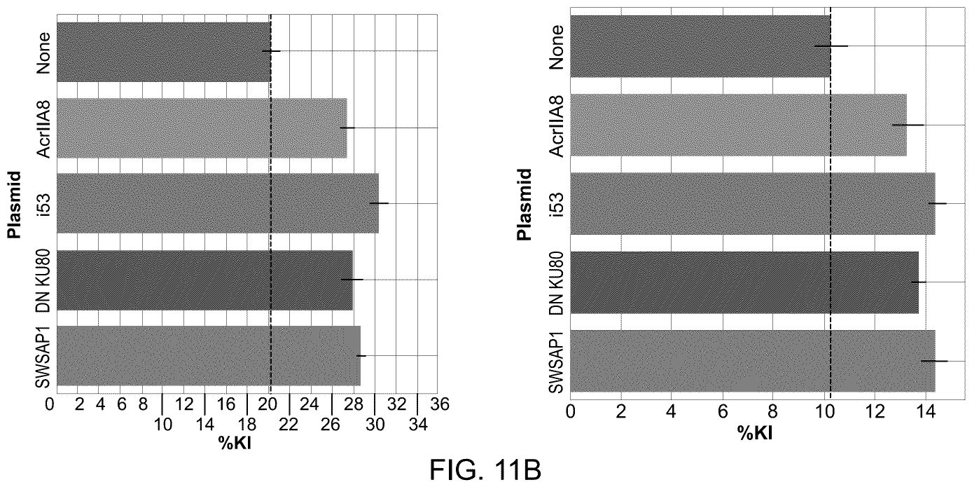

The screening method identified three genes that improved CAR and GFP marker knock in (KI) in T cells: SWSAP1, a RAD51 paralog; dominant negative KU80; and AcrIIA8-CDT1 (FIGS. 10A and 10B). FIG. 10A shows the correlation in KI efficiency between two donor cell lines electroporated with plasmid transiently expressing the indicated proteins. FIG. 10B shows the correlation in KI efficiency between two donor cell lines electroporated with plasmid transiently expressing the indicated proteins. Luciferase was used as a control. NLS tags are noted where applicable. Two anti-CRISPR candidate proteins, AcrIIA8 and AcrIIA10, demonstrated enhanced KI as compared to other anti-CRISPRs AcrII4 and AcrII5. FIG. 11A shows bar graphs of the % KI efficiency between cells from two donors using a 9 kb plasmid with a GFP reporter. FIG. 11B shows bar graphs of the % KI efficiency between cells from two donors using a 9 kb plasmid with an exemplary myc-tagged CAR. In both cases a control plasmid encoding a luciferase reporter gene (luc) was also used. As shown in both FIGS. 11A and 11B adding a small non-replicative, non-integrating plasmid encoding i53, dominant negative (DN) KU80, SWSAP1, or AcrIIA8 enhanced the knock in (KI) efficiency of the GFP or exemplary CAR gene.

SWSAP1 expression has not previously been known to affect HDR transgene integration performance and thus the improved KI efficacy in cells expressing SWSAP1 was unexpected. Dominant negative KU80 is a fragment of KU80 that inhibits formation of the KU70/KU80 complex, which is involved in NHEJ. AcrIIA8-CDT1 is a fusion of a Cas9 anti-CRISPR (AcrIIA8) to a fragment of CDT1. AcrIIA8-CDT1 acts as a degron specific to S and G2 cell cycle phases when HDR occurs. When the anti-CRISPR is degraded, it frees the Cas9 RNP to cut at the appropriate cell cycle stage. Without wishing to be bound by theory, these three genes may improve KI efficiency via increasing HDR of the target gene by biasing the cellular machinery towards homologous recombination.

### Example 5: Increased Editing Efficiency with Histone Deacetylase Inhibition

The integration of transgenes with the CRISPR-Cas9 platform is inefficient and the correct transgene only gets inserted into a fraction of the input cells. This inefficiency is further exacerbated with increasing size of gene inserts. This causes problems for multiple reasons; the majority of the cells that come out of the process do not have the inserted gene and cannot function as intended, and the absolute number of cells that have the inserted gene and can function as intended can be small. These can cause issues clinically because it requires much larger cell infusions to get the intended dose of edited cells, which can increase the risk of complications. Additionally this can also cause difficulty in manufacturing sufficient numbers of edited cells to meet dose requirements. This example shows improvement in an electroporation (EP)-mediated CRISPR-Cas9 gene editing process by inhibiting histone deacetylases (HDAC) with various compounds to increase the knock-in efficiency and edited cell yield from the same input. Multiple different HDAC inhibitors can be effective for these purposes.

### Materials and Methods

Freshly isolated or frozen and thawed isolated T cells were activated with CD3/CD28 Dynabeads (Thermo Fisher Scientific, 43500D or 40203D) at either a 3:1 or 2:1 bead:cell ratio for 2 days in complete media (TexMACS (Miltenyi Biotec, 170-076-306)+3% CellVive (Biolegend, 420502) or Human Serum (GeminiBio, 100512)+12.5 ng/ml IL-7 +12.5 ng/mL IL-15) in GRex culture flasks (Wilson Wolf Manufacturing, RU81100) at 37° C., 5% CO2, or complete media containing 0.1 mM sodium phenylbutyrate. Beads were then removed and cells were pelleted and resuspended into Genome Editing Buffer (Thermo Fisher Scientific, A4998002 or A4998001), plasmid DNA and ribonucleoprotein (RNP) were then added to the cells and the cells were electroporated using the CTS Xenon Electroporation System (Thermo Fisher Scientific) using a Xenon SingleShot (Thermo Fisher Scientific, A50305). Post-electroporation, cells were transferred to GRex plates (Wilson Wolf Manufacturing, 80660 MS) containing complete media, or complete media with HDAC inhibitor (0.5 mM, 1 mM, 2 mM, or 4 mM sodium phenylbutyrate, 0.016 μM Quisinostat, or 0.0375 μM Panobinostat) or other inhibitor compounds, at 1/10th the fill volume of the vessel. One day post-electroporation, complete media was added to full volume. Cells were then sampled for cell counts and flow cytometry to measure percent knock-in at 4 to 7 days post-electroporation.

### Results

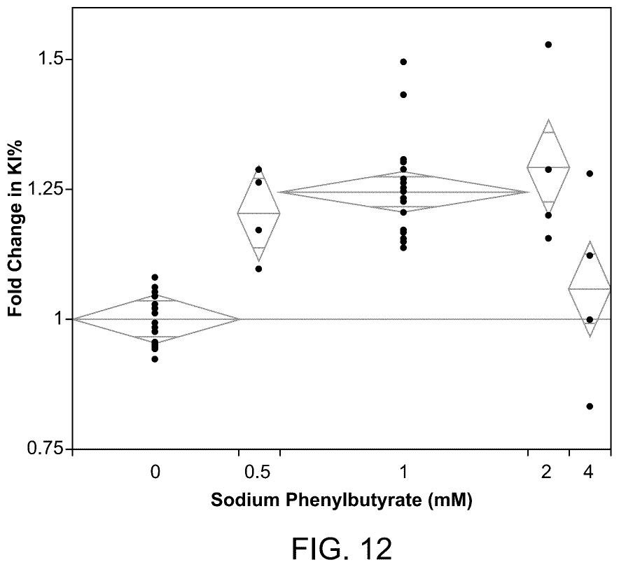

As shown in FIG. 12, incubation of the T cells with 0.5 mM, 1 mM, 2 mM and 4 mM sodium phenylbutyrate with 1% DMSO in culture media after electroporation increased the knock in (KI) efficiency as compared to media with no sodium phenylbutyrate. The 1 mM sodium phenylbutyrate condition resulted in the best % KI.

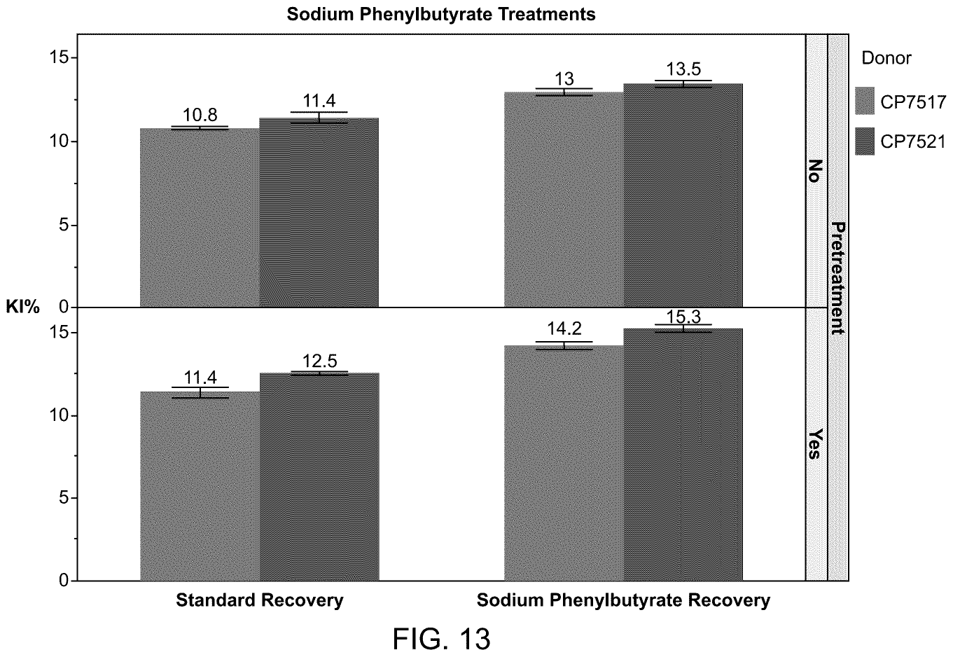

Sodium phenylbutyrate can also be used as a pre-treatment where a concentration of 0.1 mM is included in the culture media for 2 days prior to EP. This concentration and duration of treatment provided an increase in transgene knock-in (KI %) as compared to a standard recovery with no sodium phenylbutyrate (FIG. 13). This effect has shown to be additive to the effect of recovery treatment, with a combination of both pre-treatment and post-treatment with sodium phenylbutyrate giving better knock-in improvement than either alone.

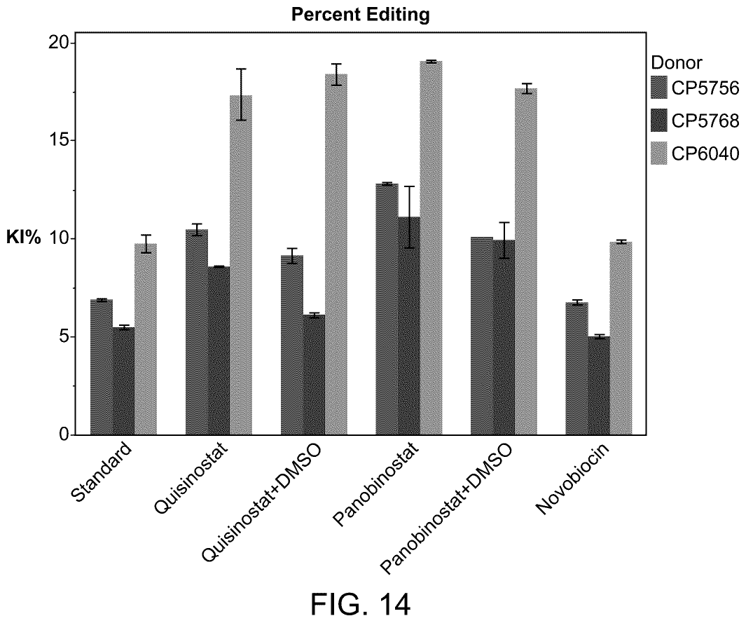

As shown in FIG. 14, incubation of the T cells with 0.016 μM Quisinostat and 0.0375 μM Panobinostat after electroporation also increased the knock in (KI) efficiency as compared to media with no Quisinostat or Panobinostat (FIG. 14). The addition of 1% DMSO did not affect the KI % of the transgene. Interestingly, novobiocin did not show improved KI % in the electroporation knock in method as compared to standard electroporation.

In sum, sodium phenylbutyrate, quisinostat, and panobinostat improved CRISPR editing knock-in of a transgene after electroporation at concentrations that resulted in low toxicity but also maintained a high edited cell yield (e.g., no loss in T cell output). Furthermore, sodium phenylbutyrate show both an average of a 67% improvement in KI % efficiency, and can used as both a pre-electroporation treatment or post-electroporation treatment, across multiple electroporation platforms.

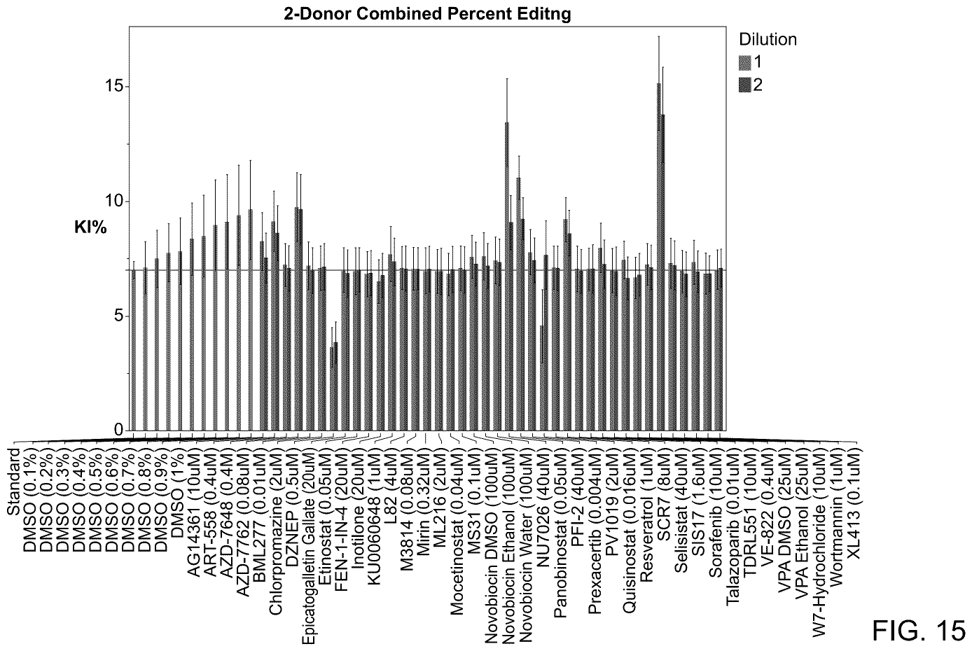

A select number of additional inhibitor compounds also increased transgene KI %. For example, ATR inhibitors such as VE-822 can provide a substantial increase in knock-in. Other compounds that improved editing efficiency included AG14361 a PARP-1 inhibitor, ART-558 a Pole inhibitor, and AZD7762, a CHK inhibitor. DNA-Pk inhibitors such as KU0600648 and NU7026 also showed improvements in editing efficiency (FIG. 15). In addition, many compounds that have previously been identified as improving knock-in or which may theoretically have a function that should suppress non-homologous end joining to promote efficient gene insertion do not show an improvement.

### Example 6: Increased Editing Efficiency with Increased Electroporation Pulses

### Materials and Methods

### Five Pulse Process Development

Standard protocol: Freshly isolated or frozen and thawed isolated T cells were activated with CD3/CD28 Dynabeads (Thermo Fisher Scientific, 43500D or 40203D) for 2 days in complete media (TexMACS+xeno-free Supplement B or xeno-free Supplement A) at 37° C., 5% CO2. Beads were then removed and 100×10⁶ cells were pelleted and resuspended into 170 mM glycerol, 2.5 mM NAC buffer and Gene Editing buffer with a final volume of 1 mL electroporation reaction after RNP and DNA were added. The cells were then incubated for 10 minutes at room temperature. 40 μg plasmid DNA expressing an exemplary transgene (e.g., a heterologous protein, such as a priming receptor and/or a CAR) and ribonucleoprotein (RNP) were then added to the cells. The cells were electroporated at 2300V for 3 ms per pulse, for four pulses.

Five pulse protocol: Freshly isolated or frozen and thawed isolated T cells were activated with CD3/CD28 Dynabeads (Thermo Fisher Scientific, 43500D or 40203D) for 2 days in complete media (TexMACS+xeno-free Supplement B or xeno-free Supplement A) at 37° C., 5% CO2. Beads were then removed and cells were pelleted and resuspended into 170 mM glycerol, 2.5 mM NAC buffer and Gene Editing buffer with a final volume of 1 mL electroporation reaction after RNP and DNA were added. The cells were then incubated for 10 minutes at room temperature. 20 μg plasmid DNA expressing an exemplary transgene (e.g., a heterologous protein, such as a priming receptor and/or a CAR) and ribonucleoprotein (RNP) were then added to the cells. The cells were electroporated at 2300V for 3 ms per pulse, for five pulses.

The DNA insertion cassette in the DNA plasmid was 8.2 kb. A nanoplasmid with a reduced backbone length as compared to the full plasmid backbone was also generated and used in the electroporation protocol. RNP with no DNA was used as a negative control.

### Functional Assays

On Day 9 edited T cells from two donors generated form either the standard protocol or the 5 pulse protocol were tested in replicate at a 1:50 or 1:100 E:T cell ratio using tumor cells expressing the cognate ligand for the exemplary transgene construct (e.g., the priming receptor and/or CAR). The number of live target cells after incubation with the T cells was assessed.

### Cell Expansion Assay

Cells from 5 unique donors were electroporated with an exemplary transgene construct (e.g., a CAR) at 100M input number using the 4 and 5-pulse conditions. The cells were counted on D7 and D9. Both the KI % and the total edited cell number was collected and graphed.

### Results

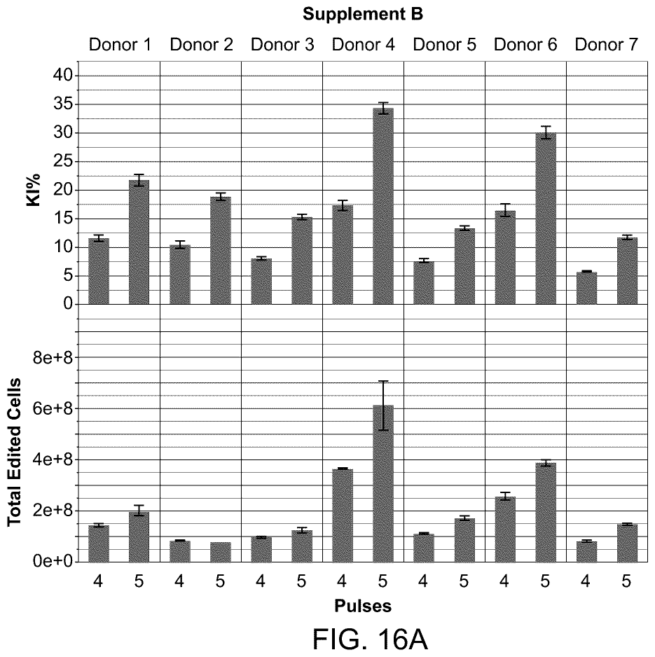

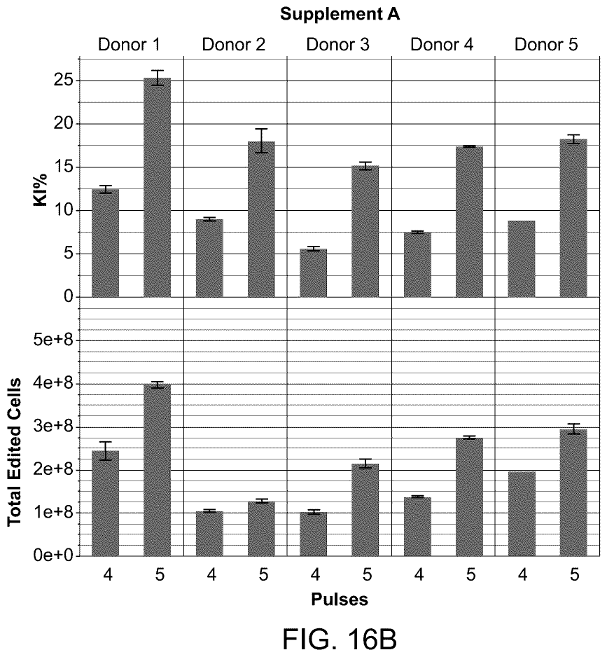

FIG. 16A shows the % knock in (KI) and total edited T cells from seven different donor expressing the exemplary transgene(s) after 4 or 5 electroporation pulses in media supplemented with xeno-free Supplement B. The 4 pulse sample is shown in the left bar, the 5 pulse sample is shown in the right bar. FIG. 16B shows the % knock in (KI) and total edited T cells from five different donor expressing the exemplary transgene(s) after 4 or 5 electroporation pulses in media supplemented with xeno-free Supplement A. The 4 pulse sample is shown in the left bar, the 5 pulse sample is shown in the right bar. The 5-pulse process had increased editing efficiency as compared to the 4-pulse process in all conditions tested with respect to KI % and produced equivalent or higher total edited cell number as compared to the 4-pulse process. Thus, the 5-pulse process improved the KI % and total edited cell number. Furthermore, less DNA template was used in the electroporation protocol using 5 pulses (20 μg) as compared to the standard protocol using 4 pulses (40 μg). Thus, the 5 pulse method allows for improved KI % and increased total edited cell numbers while requiring less starting DNA template.

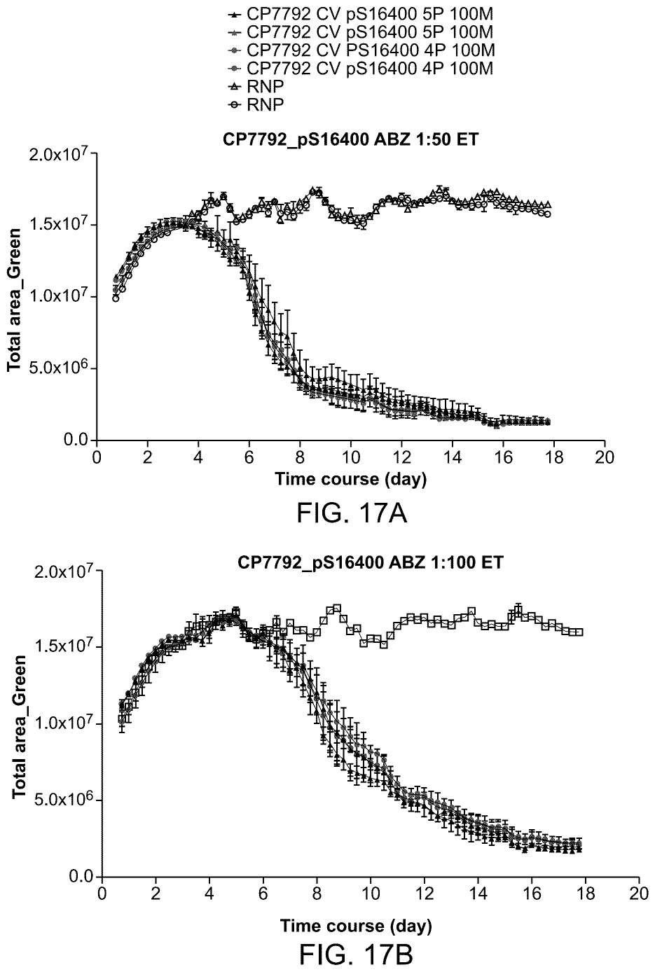

T cell functional data showed equivalent anti tumor activity of T cells across the tested pulse conditions. FIG. 17A shows the number of tumor target cells after incubation with edited T cells at a 1:50 E:T ratio. FIG. 17B shows the number of tumor target cells after incubation with edited T cells at a 1:100 E:T ratio. T cells from a second donor were also tested and showed the same results (data not shown). In both figures the T cells were electroporated with either 4 pulses or 5 pulses. As shown in FIGS. 17A and 17B, through an 18 day time course assay the T cells edited with the 5-pulse condition showed similar tumor clearance (e.g., anti-tumor efficacy) as compared to T cells edited with 4-pulse condition. Thus, there was no functional difference between T cell function in the 4 pulse or 5 pulse conditions and the additional pulse in the 5 pulse protocol did not adversely affect the functionality of the T cells generated with the 5-pulse condition.

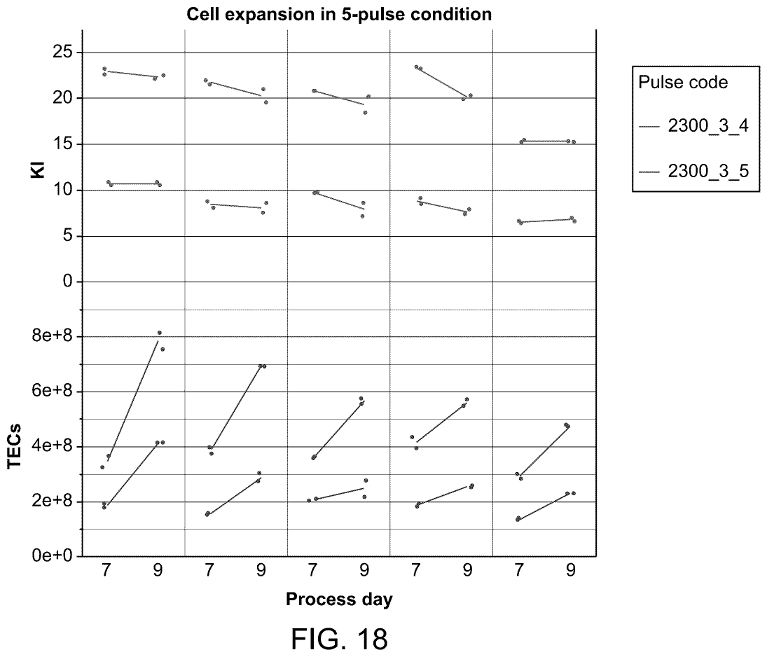

As shown in FIG. 18, % KI and cell expansion (TECs) on Days 7 and 9 post electroporation was increased in the 5-pulse protocol as compared to the 4-pulse protocol in T cells from five donors. The upper line in each bar indicates the 5 pulse condition, while the lower line indicates the 4 pulse condition.

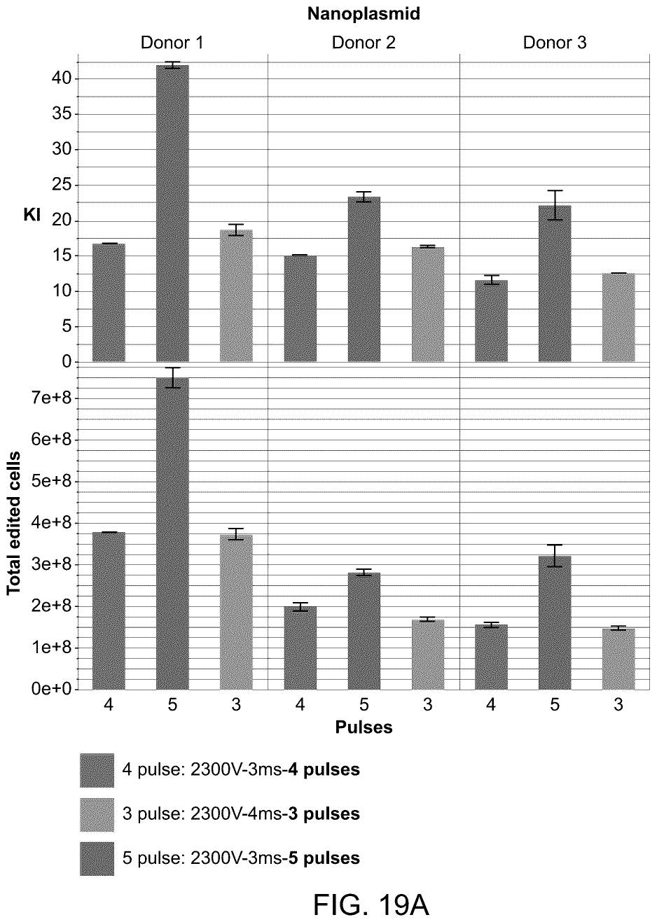

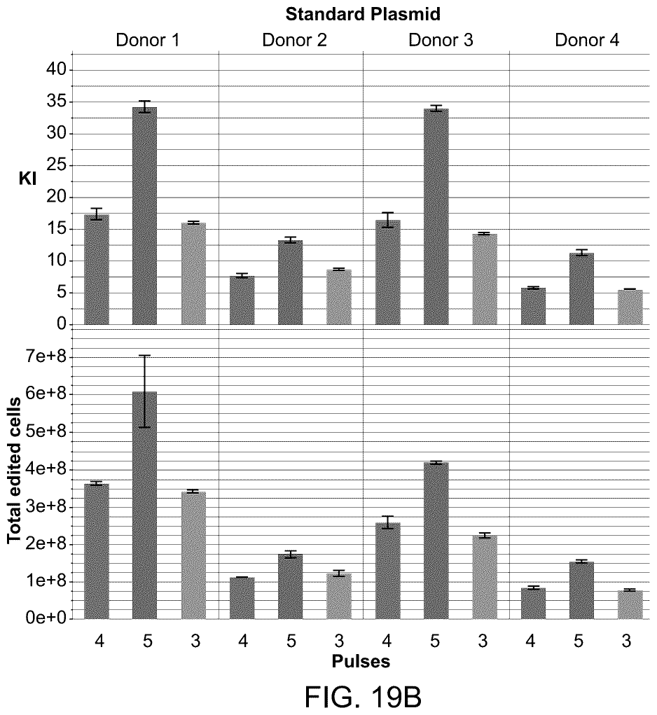

The % KI and total edited cells in the 5 pulse protocol as compared to the 3 pulse and 4 pulse protocol were assessed with different DNA sizes (a nanoplasmid and the standard plasmid). As shown in FIGS. 19A and 19B, the 5 pulse method (middle bar) increased % KI and total edited T cells as compared to the 4 pulse method (left bar) and 3 pulse method (right bar).

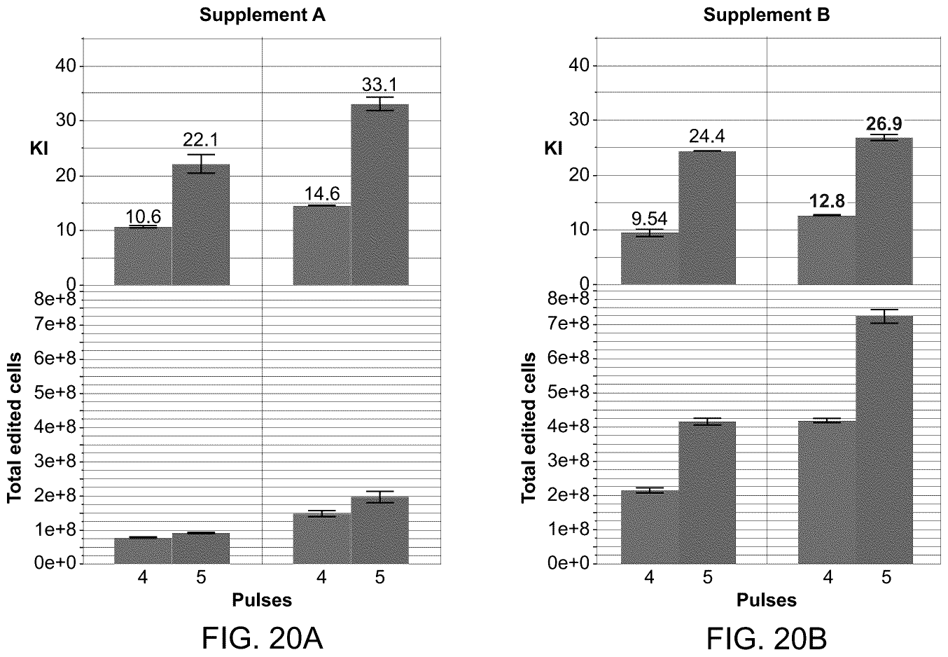

The 5 pulse protocol also increased transgene KI efficiency of a second exemplary transgene that was inducibly expressed. As shown in FIGS. 20A and 20B, the 5 pulse protocol resulted in an improvement in KI of nearly 2-fold in T cells from four donors incubated with the xeno-free media supplements A and B. Thus, the KI improvement observed with the 5-pulse process was not contingent on the presence of a constitutively expressed transgene.

TABLE 2
shows the starting, mid-process and endpoint T cell counts of an exemplary T cell editing assay. “Mid range,”
“well provisioned,” and “poorly provisioned” refer to the number of T cells initially isolated
from a donor sample via leukapheresis (e.g., the T cells obtained from a Leukopak), with fewer T cells obtained
as a starting material in the “poorly provisioned” and “mid range” samples as compared to the “well provisioned” sample.

| | | KI+ | KI+ | | Cells | Cells | | | |
|---|---|---|---|---|---|---|---|---|---|
| | T cell | cells | cells | Cells | remaining | remaining | | Meets | Meets |
| | Input | from 4 | from 5 | needed | after QC - | after QC - | Cells | needs - | needs - |
| | per EP | pulse | pulse | for QC | 4 pulse | 5 pulse | needed | 4 pulse? | 5 pulse? |
| Well | 1.00E+08 | 3.51E+08 | 7.05E+08 | 3.00E+08 | 5.08E+07 | 4.05E+08 | 3.00E+07 | Yes | Yes |
| provisioned | | | | | | | | | |
| Mid range | 1.00E+08 | 3.17E+08 | 7.38E+08 | 3.00E+08 | 1.74E+07 | 4.38E+08 | 6.00E+07 | Yes, with | Yes |
| | | | | | | | | duplicates | |
| Poorly | 5.00E+07 | 2.15E+08 | 4.99E+08 | 3.00E+08 | −8.54E+07 | 1.99E+08 | 6.00E+07 | No | Yes |
| provisioned | | | | | | | | | |

As shown in Table 2, when starting with a lower anticipated T cell number from the leukapheresis (“mid range”), the input T cell number may be insufficient to perform electroporation even when using 100×10⁸ T cell input. Performing the electroporation with a 50×10⁶ T cell input (poorly provisioned) with the 4 pulse condition is unlikely to yield sufficient edited cell numbers to meet the requirements for in vivo studies. This limitation can be overcome using the 5-pulse protocol that results in increased edited T cells as compared to the 4 pulse protocol. Thus, the 5-pulse protocol results in an improved ability to meet in vivo studies T cell dose requirements.

In sum, the 5-pulse process improved KI % and total edited cells across all donors tested. This data was recapitulated in multiple assays. Functional studies of the cell indicated no significant difference in performance of the electroporated cells at either 1:50 or 1:100 E:T ratio. ROS is generated during the electroporation and leads to an increase in toxicity associated with the electroporation process. Without wishing to be bound by theory, incorporation of NAC improves ROS scavenging, along with other optimizations described herein, and enabled an additional pulse in the electroporation program. Thus, a fifth electroporation pulse improved the KI % and total edited cell number.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition comprising a solution comprising an RNase inhibitor, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

2. A composition comprising a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

3. A composition comprising a solution comprising an osmolarity-adjusting agent, a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of a cell.

4.-9. (canceled)

10. The composition of claim 2, wherein the N acetyl-L-cysteine (NAC) is present in the solution at a final concentration of between about 1-10 mM.

11. The composition of claim 10, wherein the N acetyl-L-cysteine (NAC) is present in the solution at a final concentration of about 2.5 mM.

12.-30. (canceled)

31. The composition of claim 2, wherein the nuclease domain comprises a CRISPR-associated endonuclease (Cas), optionally a Cas9 nuclease.

32. (canceled)

33. The composition of claim 2, wherein the size of the DNA template is greater than or equal to about 0.3 kb, 0.5 kb, 1.0 kb, 1.5 kb, 2.0 kb, 2.5 kb, 3.0 kb, 3.5 kb, 4.0 kb, 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, 10.1 kb, 10.2 kb, 10.3 kb, 10.4 kb, 10.5 kb, 10.6 kb, 10.7 kb, 10.8 kb, 10.9 kb, 11.0 kb, 11.1 kb, 11.2 kb, 11.3 kb, 11.4 kb, 11.5 kb, 11.6 kb, 11.7 kb, 11.8 kb, 11.9 kb, 12.0 kb, 12.1 kb, 12.2 kb, 12.3 kb, 12.4 kb, 12.5 kb, 12.6 kb, 12.7 kb, 12.8 kb, 12.9 kb, 13.0 kb, or larger, or any size DNA template in between these sizes.

34. (canceled)

35. The composition of claim 2, further comprising a cell comprising the genomic sequences flanking the insertion site in the genome of the cell.

36. The composition of claim 35, wherein the cell is a mammalian cell, a human cell, a hematopoietic cell, an immune cell, a primary immune cell, or a primary human immune cell.

37. (canceled)

38. The composition of claim 36, wherein the immune cell is a natural killer (NK) cell, a T cell, a CD8+ T cell, a CD4+ T cell, a primary T cell, or a T cell progenitor.

39.-48. (canceled)

49. The composition of claim 2, wherein the insertion site target region of the genome of the cell is a T Cell Receptor Alpha Constant (TRAC) locus or a genomic safe harbor (GSH).

50. The composition of claim 49, wherein the safe harbor locus is selected from any one of the integration sites designated: GS94, GS88, GS89, GS90, GS91, GS92, GS93, GS95, GS96, GS97, GS98, GS99, GS100, GS101, GS102, GS103, GS104, GS105, GS106, GS107, GS108, GS109, GS110, GS111, GS112, GS113, GS114, GS115, GS116, GS117, GS118, GS119, or GS120.

51. (canceled)

52. The composition of claim 49, wherein the safe harbor locus is selected from: chr11:128340000-128350000, chr10:33130000-33140000, chr10:72290000-72300000, chr11:65425000-65427000 (NEAT1), chr15:92830000-92840000, chr16:11220000-11230000, chr2:87460000-87470000, chr3:186510000-186520000, chr3:59450000-59460000, chr8:127980000-128000000, or chr9:7970000-7980000.

53. (canceled)

54. The composition of claim 2, comprising one or more gRNAs comprising any one of SEQ ID NOS: 1-120.

55.-57. (canceled)

58. The composition of claim 2, wherein the DNA template comprises a heterologous sequence.

59.-61. (canceled)

62. The composition of claim 2, wherein the DNA template comprises a chimeric antigen receptor (CAR) and/or a priming receptor comprising a transcription factor.

63.-67. (canceled)

68. The composition of claim 62, wherein the priming receptor comprises, in an N terminus to C terminus direction:
a. an extracellular antigen-binding domain having a binding affinity for an antigen;
b. a transmembrane domain comprising one or more ligand-inducible proteolytic cleavage sites; and
c. an intracellular domain comprising a human or humanized transcriptional effector, wherein binding of an antigen to the extracellular antigen-binding domain results in cleavage at the ligand-inducible proteolytic cleavage site thereby releasing the intracellular domain.

69.-70. (canceled)

71. The composition of claim 62, wherein the CAR comprises, from N-terminus to C-terminus,
a. an extracellular antigen-binding domain having a binding affinity for an antigen;
b. a transmembrane domain;
c. an intracellular co-stimulatory domain; and
d. an intracellular activation domain.

72.-112. (canceled)

113. A method of editing a cell, comprising:
a. providing a solution comprising N acetyl-L-cysteine (NAC), a ribonucleoprotein complex (RNP), and a DNA template, wherein the RNP comprises a nuclease domain and a guide RNA, and wherein the 5′ and 3′ ends of the DNA template comprise nucleotide sequences that are homologous to genomic sequences flanking an insertion site in the genome of the cell; and
b. editing the cell via insertion of the DNA template into the insertion site in the genome of the cell.

114.-216. (canceled)

217. A method of treating a subject having or at risk of having a disease, comprising:
a. conducting the method of claim 113; and
b. administering to the subject an effective amount of a composition comprising the cell or a population thereof.

218. (canceled)

219. (canceled)

220. (canceled)
